(19) United States
(12) Patent Application Publication
(10) Pub. No.: US 2009/0305310 A1
(43) Pub. Date: Dec. 10, 2009

(54) Methods for Determining Notch Signaling and Uses Thereof

(75) Inventor: Cedric S. Wesley, South Burlington, VT (US)

Correspondence Address: WOLF GREENFIELD & SACKS, P.C., 600 ATLANTIC AVENUE, BOSTON, MA, 02210-2206, US

(73) Assignee: University of Vermont and State Agricultural College, Burlington, VT (US)

(21) Appl. No.: 12/085,425

(22) PCT Filed: Nov. 20, 2006
(86) PCT No.: PCT/US2006/044974

§ 371 Date: Jul. 7, 2009

Related U.S. Application Data
(60) Provisional application No. 60/738,811, filed on Nov. 22, 2005.

(51) Int. Cl. G01N 33/53

(52) U.S. Cl. 435/7.21

(57) ABSTRACT

The invention relates, in part, to methods of determining Notch signaling in cells, tissues and/or subjects. The invention additionally relates, in part, to diagnostic assays for cell differentiation-associated diseases or conditions and for screening tools in research and clinical applications.

### RELATED APPLICATIONS

This application claims priority under 35 U.S.C. §119(e) from U.S. provisional application Ser. No. 60/738,811, filed Nov. 22, 2005, the entire content of which is incorporated by reference herein.

### GOVERNMENT SUPPORT

This invention was made in part with government support under grant number NS043122 from the National Institute of Neurological Disorders and Stroke (NINDS). The United States Government may have certain rights in this invention.

### FIELD OF THE INVENTION

The invention relates, in part, to methods of determining Notch signaling in cells, tissues and/or subjects. The invention additionally relates, in part, to diagnostic assays for cell differentiation-associated diseases or conditions and for screening tools in research and clinical applications.

### BACKGROUND OF THE INVENTION

Notch signaling regulates the differentiation of almost all tissues in all animals from worms to humans. The Notch signaling pathway is a highly conserved, basic signaling pathway. Loss or abnormal Notch signaling has been linked to numerous cancers, birth defects, and neurological diseases including dementia, stroke, and Alzheimer's. It is generally believed in the field that the distribution of the Notch receptor producing this signaling is uniform and featureless during development. It has been notoriously difficult to identify in vivo the level of Notch signaling because very small quantities appear to be sufficient for normal or abnormal functions. Some target genes of this signaling have been identified but their expression is very context dependent and subject to feedback regulation. In some instances, an increased level of Notch is associated with advanced stages of malignancy or diseased states but this becomes apparent very late in the process when little can be done. Furthermore, it is not clear whether this increased level is due to gain or loss of Notch signaling.

### SUMMARY OF THE INVENTION

The invention relates in part to the surprising discovery that Notch is cleaved to produce truncated Notch polypeptides that act as dominant negative molecules, and that these dominant-negative molecules act as part of an auto-down-regulatory mechanism for Notch signaling. It has now been discovered that the level of truncated Notch polypeptides and the ratio of the amounts of these truncated Notch polypeptides to the amount of full-length Notch polypeptides are useful to determine the level of Notch signaling in cells, tissues, and subjects. The level of Notch signaling in cells and tissues is known to be involved in features of cell and tissue differentiation and the maintenance of cell identity. These features are involved in normal cell differentiation and maintenance as well as abnormal cell differentiation and maintenance. Thus, the amount of truncated Notch polypeptide in a cell or tissue or the ratio of the amount of truncated Notch polypeptide to full-length polypeptide can be used to determine the level of Notch signaling in the cell or tissue. The invention includes, in part, methods of determining levels of Notch signaling and the use of such determinations for diagnosing cell differentiation-associated and/or cell maintenance-associated diseases or conditions, screening pharmacological compounds for Notch-signaling activity, and cell, tissue, and animal models of cell differentiation-associated and/or cell maintenance-associated disease or conditions.

According to one aspect of the invention, methods for identifying the level of Notch signaling in a cell or tissue are provided. The methods include determining an amount of truncated Notch polypeptide of the cell or tissue, comparing the amount of truncated Notch polypeptide of the cell or tissue to an amount of truncated Notch polypeptide of a control cell or tissue, wherein a higher or lower amount of truncated Notch polypeptide of the cell or tissue compared to the control cell or tissue identifies the cell or tissue as having a different level of Notch signaling than the level of Notch signaling of the control cell or tissue. In some embodiments, a higher amount of truncated Notch polypeptide in the cell or tissue compared to the control cell or tissue identifies the cell or tissue as having a lower level of Notch signaling than the control cell or tissue. In other embodiments, a lower amount of truncated Notch polypeptide in the cell or tissue compared to the control cell or tissue identifies the cell or tissue as having a higher level of Notch signaling than the control cell or tissue. In some embodiments, determining the amount of truncated Notch polypeptide comprises the use of immunodetection methods. In certain embodiments, the amount of truncated Notch polypeptide is determined by contacting the cell or tissue with one or more antibodies or antigen-binding fragments thereof that specifically bind to one or more domain(s) present in a truncated Notch polypeptide and one or more antibodies or antigen-binding fragments thereof, that specifically bind to the C-terminal domain of a Notch polypeptide, detecting the level of binding of the antibodies or antigen-binding fragments thereof to the cell or tissue, and comparing the level of binding of the antibodies or antigen-binding fragments thereof that bind to domain(s) present in the truncated Notch polypeptide to the level of binding of the antibodies or antigen-binding fragments thereof that bind to the C-terminal domain of the Notch polypeptide as a determination of the amount of truncated Notch polypeptide of the cell or tissue. In some embodiments, the one or more antibodies or antigen-binding fragments thereof that specifically bind to a domain present in a truncated Notch polypeptide is an antibody or antigen-binding fragment thereof that specifically binds either the extracellular domain of the Notch polypeptide or a Ram23+Ankyrin domain of the Notch polypeptide. In some embodiments, the cell or tissue is contacted with least one antibody or antigen-binding fragment thereof that specifically binds to the C-terminal domain of a Notch polypeptide and at least one antibody or antigen-binding fragment thereof that specifically binds to the extracellular Notch polypeptide domain or to the Ram23+Ankyrin Notch polypeptide domain. In some embodiments, the truncated Notch polypeptide is a Notch polypeptide without an extracellular Notch polypeptide domain and without a C-terminal Notch polypeptide domain. In certain embodiments, the truncated Notch polypeptide is a Notch polypeptide without a transcription activating domain (TAD). In some embodiments, the truncated Notch polypeptide is a Notch polypeptide without a Ram23+Ankyrin Notch polypeptide domain and without a C-terminal Notch polypeptide domain. In some embodiments, the truncated Notch polypeptide consists of a Notch polypeptide extracellular domain. In some embodiments, the cell or tissue is an invertebrate cell or tissue. In some embodiments, the cell or tissue is a vertebrate cell or tissue. In certain embodiments, the Notch polypeptide is a vertebrate Notch polypeptide. In some embodiments, the Notch polypeptide is an invertebrate Notch polypeptide. In some embodiments, the vertebrate is a mammal. In some embodiments, the mammal is a human. In some embodiments, the Notch polypeptide is a human Notch 1, human Notch 2, human Notch 3, or human Notch 4 polypeptide. In certain embodiments, the antibodies or antigen-binding fragments thereof, are detectably labeled. In certain embodiments, the detectable label is a fluorescent, enzyme, radioactive, metallic, biotin, chemiluminescent, or bioluminescent label.

According to another aspect of the invention, methods for identifying the level of Notch signaling in a cell or tissue are provided. The methods include determining a ratio of an amount of truncated Notch polypeptide of the cell or tissue to an amount of full-length Notch polypeptide of the cell or tissue, comparing the ratio of the amount of truncated Notch polypeptide to the amount of full-length polypeptide of the cell or tissue to a ratio of the amount of truncated Notch polypeptide to the amount of full-length Notch polypeptide of a control cell or tissue, wherein a different ratio of the cell or tissue compared to the ratio of the control cell or tissue identifies the cell or tissue as having a different level of Notch signaling than the level of Notch signaling of the control cell or tissue. In some embodiments, a higher ratio of the amount of truncated Notch polypeptide to the amount of full-length polypeptide of the cell or tissue compared to the ratio of the amount of truncated Notch polypeptide to the amount of full-length polypeptide of the control cell or tissue identifies the cell or tissue as having a lower level of Notch signaling than the control cell or tissue. In some embodiments, a lower ratio of amount of truncated Notch polypeptide to the amount of full-length polypeptide of the cell or tissue compared to the ratio of the amount of truncated Notch polypeptide to the amount of full-length polypeptide of the control cell or tissue identifies the cell or tissue as having a higher level of Notch signaling than the control cell or tissue. In some embodiments, the truncated Notch polypeptide is a Notch polypeptide without an extracellular Notch polypeptide domain and without a C-terminal Notch polypeptide domain. In certain embodiments, the truncated Notch polypeptide is a Notch polypeptide without a transcription activating domain (TAD). In some embodiments, the truncated Notch polypeptide is a Notch polypeptide without a Ram23+Ankyrin Notch polypeptide domain and without a C-terminal Notch polypeptide domain. In some embodiments, the truncated Notch polypeptide consists of a Notch polypeptide extracellular domain. In some embodiments, determining the ratio of the amount of truncated Notch polypeptide of the cell or tissue to the amount of full-length Notch polypeptide of the cell or tissue comprises the use of immunodetection methods. In some embodiments, determining the ratio of the amount of the truncated Notch polypeptide of the cell or tissue to the amount of the full-length Notch polypeptide of the cell or tissue includes contacting the cell or tissue with one or more antibodies or antigen-binding fragments thereof that specifically bind to one or more domain(s) present in the truncated Notch polypeptide, contacting the cell or tissue with one or more antibodies or antigen-binding fragments thereof that specifically bind the C-terminal domain of the Notch polypeptide; detecting the level of binding of the truncated Notch polypeptide and Notch polypeptide C-terminal antibodies or antigen-binding fragments thereof in the cell or tissue, and comparing the level of binding of the truncated Notch polypeptide and Notch polypeptide C-terminal antibodies or antigen-binding fragments thereof to the cell or tissue to determine the ratio of the truncated and full-length Notch polypeptide in the cell or tissue. In some embodiments, the cell or tissue is contacted with least one antibody or antigen-binding fragment thereof that specifically binds to the C-terminal domain of a Notch polypeptide and at least one antibody or antigen-binding fragment thereof that specifically binds to the extracellular Notch polypeptide domain or to the Ram23+Ankyrin Notch polypeptide domain. In certain embodiments, the cell or tissue is a vertebrate cell or tissue. In some embodiments, the cell or tissue is an invertebrate cell or tissue. In some embodiments, the Notch polypeptide is a vertebrate Notch polypeptide. In certain embodiments, the Notch polypeptide is an invertebrate Notch polypeptide. In some embodiments, the vertebrate is a mammal. In some embodiments, the mammal is a human. In some embodiments, the Notch polypeptide is a human Notch 1, human Notch 2, human Notch 3, or human Notch 4 polypeptide. In certain embodiments, the antibodies or antigen-binding fragments thereof are detectably labeled. In some embodiments, the detectable label is a fluorescent, enzyme, radioactive, metallic, biotin, chemiluminescent, or bioluminescent label. In some embodiments, the detectable label is a fluorescent label.

According to yet another aspect of the invention, methods of diagnosing a cell differentiation-associated and/or cell maintenance-associated disease or condition in a cell or tissue are provided. The methods include determining an amount of truncated Notch polypeptide in the cell or tissue; comparing the amount of truncated Notch polypeptide to an amount of truncated Notch polypeptide in a control cell or tissue, wherein a difference in the amount of truncated Notch polypeptide in the cell or tissue compared to the amount of truncated Notch polypeptide in the control cell or tissue is diagnostic for the cell differentiation-associated and/or cell maintenance-associated disease or condition in the cell or tissue. In some embodiments, a higher amount of truncated Notch polypeptide of the cell or tissue compared to the amount of truncated Notch polypeptide of the control cell or tissue identifies the cell or tissue as having a lower level of Notch signaling than the control cell or tissue and is diagnostic for cell differentiation-associated and/or cell maintenance-associated disease or condition in which Notch signaling is reduced compared to the level of Notch signaling in a cell or tissue that is free of the cell differentiation-associated and/or cell maintenance-associated disease or condition. In certain embodiments, a lower amount of truncated Notch polypeptide of the cell or tissue compared to the amount of truncated Notch polypeptide of the control cell or tissue identifies the cell or tissue as having a higher level of Notch signaling than the control cell or tissue and is diagnostic for cell differentiation-associated and/or cell maintenance-associated disease or condition in which Notch signaling is increased compared to the level of Notch signaling in a cell or tissue that is free of the cell differentiation-associated and/or cell maintenance-associated disease or condition. In some embodiments, determining the amount of truncated Notch polypeptide in the cell or tissue comprises the use of immunodetection methods. In some embodiments, the amount of truncated Notch polypeptide is determined by contacting the cell or tissue with one or more antibodies, or antigen-binding fragments thereof, that specifically bind to one or more domains present in a truncated Notch polypeptide and one or more antibodies or antigen-binding fragments thereof that specifically bind to the C-terminal domain of a Notch polypeptide, detecting the level of binding of the antibodies or antigen-binding fragments thereof and comparing the level of binding of the antibodies or antigen-binding fragments thereof that bind a domain present in a truncated Notch polypeptide to the binding of the antibodies or antigen-binding fragments thereof, that bind to the C-terminal domain of the Notch polypeptide as a determination of the amount of truncated Notch polypeptide of the cell or tissue. In some embodiments, the cell or tissue is contacted with least one antibody or antigen-binding fragment thereof that specifically binds to the C-terminal domain of a Notch polypeptide and at least one antibody or antigen-binding fragment thereof that specifically binds to the extracellular Notch polypeptide domain or to the Ram23+Ankyrin Notch polypeptide domain. In certain embodiments, the cell differentiation-associated and/or cell maintenance-associated disease or condition is cancer, a neurodegenerative disease, development, or cell and tissue repair. In some embodiments, the cell differentiation-associated and/or cell maintenance-associated disease or condition is cerebral autosomal dominant arteriopathy with subcortical infarcts and leukoencephalopathy (CADASIL), Allagile syndrome, leukemia (T-cell acute lymphoblastic), Spondylocostal dystosis, down syndrome, Alzheimer's disease, heart diseases, or a prion disease. In some embodiments, the one or more antibodies or antigen-binding fragments thereof that specifically bind to a truncated Notch polypeptide are antibodies or antigen-binding fragments thereof that specifically bind either the extracellular domain of the Notch polypeptide or a Ram23+Ankyrin domain of the Notch polypeptide. In certain embodiments, the truncated Notch polypeptide is a Notch polypeptide without an extracellular Notch polypeptide domain and without a C-terminal Notch polypeptide domain. In some embodiments, the truncated Notch polypeptide is a Notch polypeptide without a transcription activating domain (TAD). In some embodiments, the truncated Notch polypeptide is a Notch polypeptide without a Ram23+Ankyrin Notch polypeptide domain and without a C-terminal Notch polypeptide domain. In some embodiments, the truncated Notch polypeptide consists of a Notch polypeptide extracellular domain. In certain embodiments, the cell or tissue is a vertebrate cell or tissue. In some embodiments, the cell or tissue is an invertebrate cell or tissue. In some embodiments, the Notch polypeptide is an invertebrate Notch polypeptide. In certain embodiments, the Notch polypeptide is a vertebrate Notch polypeptide. In some embodiments, the vertebrate is a mammal. In some embodiments, the mammal is a human. In some embodiments, the Notch polypeptide is a human Notch 1, human Notch 2, human Notch 3, or human Notch 4 polypeptide. In some embodiments, the cell or tissue is obtained from a subject, and diagnosing a cell differentiation-associated and/or cell maintenance-associated disease or condition in the cell or tissue is diagnostic for the cell differentiation-associated and/or cell maintenance-associated disease or condition in the subject. In certain embodiments, the subject is an invertebrate. In some embodiments, the subject is a vertebrate. In some embodiments, the vertebrate is a mammal. In certain embodiments, the mammal is a human. In some embodiments, the antibodies or antigen-binding fragments thereof are detectably labeled. In some embodiments, the detectable label is a fluorescent, enzyme, radioactive, metallic, biotin, chemiluminescent, or bioluminescent label. In some embodiments, the detectable label is a fluorescent label.

According to another aspect of the invention, methods of diagnosing a cell differentiation-associated and/or cell maintenance-associated disease or condition in a cell are provided. The methods include determining a ratio of an amount of truncated Notch polypeptide of the cell to an amount of full-length Notch polypeptide of the cell, comparing the ratio of the amount of truncated Notch polypeptide to the amount of full-length polypeptide of the cell to a ratio of the amount of truncated Notch polypeptide to the amount of full-length Notch polypeptide of a control cell, wherein a different ratio of the cell or tissue compared to the ratio of the control cell or tissue is diagnostic for the cell differentiation-associated and/or cell maintenance-associated disease or condition in the cell or tissue. In certain embodiments, a higher ratio of the amount of truncated Notch polypeptide to the amount of full-length polypeptide of the cell or tissue compared to the ratio of the amount of truncated Notch polypeptide to the amount of full-length polypeptide of the control cell or tissue identifies the cell or tissue as having a lower level of Notch signaling than the control cell or tissue and is diagnostic for a cell differentiation-associated and/or cell maintenance-associated disease or condition in which Notch signaling is reduced compared to the level of Notch signaling in a cell or tissue that is free of the cell differentiation-associated and/or cell maintenance-associated disease or condition. In some embodiments, a lower ratio of the amount of truncated Notch polypeptide to the amount of full-length polypeptide of the cell or tissue compared to the ratio of the amount of truncated Notch polypeptide to the amount of full-length polypeptide of the control cell or tissue identifies the cell or tissue as having a higher level of Notch signaling than the control cell or tissue and is diagnostic for a cell differentiation-associated and/or cell maintenance-associated disease or condition in which Notch signaling is increased compared to the level of Notch signaling in a cell or tissue that is free of the cell differentiation-associated and/or cell maintenance-associated disease or condition. In some embodiments, the truncated Notch polypeptide is a Notch polypeptide without an extracellular Notch polypeptide domain and without a C-terminal domain. In some embodiments, the truncated Notch polypeptide is a Notch polypeptide without a transcription activating domain (TAD). In some embodiments, the truncated Notch polypeptide is a Notch polypeptide without a Ram23+Ankyrin Notch polypeptide domain and without a C-terminal Notch polypeptide domain. In certain embodiments, the truncated Notch polypeptide consists of a Notch polypeptide extracellular domain. In some embodiments, determining the ratio of the amount of truncated Notch polypeptide of the cell to the amount of full-length Notch polypeptide of the cell comprises the use of immunodetection methods. In some embodiments, determining the ratio of the amount of truncated Notch polypeptide of the cell or tissue to the amount of full-length Notch polypeptide of the cell or tissue includes contacting the cell or tissue with one or more antibodies or antigen-binding fragments thereof that specifically bind one or more domains present in a truncated Notch polypeptide, contacting the cell with one or more antibodies or antigen-binding fragments thereof that specifically bind the C-terminal domain of the Notch polypeptide; detecting the level of specific binding of domain(s) present in the truncated Notch polypeptide and Notch polypeptide C-terminal antibodies in the cell or tissue, and comparing the level of specific binding of the domain(s) present in the truncated Notch polypeptide and Notch polypeptide C-terminal antibodies or antigen-binding fragments thereof, to detect the ratio of truncated and full-length Notch polypeptide in the cell or tissue. In certain embodiments, the cell or tissue is contacted with least one antibody or antigen-binding fragment thereof that specifically binds to the C-terminal domain of a Notch polypeptide and at least one antibody or antigen-binding fragment thereof that specifically binds to the extracellular Notch polypeptide domain or to the Ram23+Ankyrin Notch polypeptide domain. In some embodiments, the cell or tissue is an invertebrate cell or tissue. In some embodiments, the cell or tissue is a vertebrate cell or tissue. In some embodiments, the Notch polypeptide is an invertebrate Notch polypeptide. In certain embodiments, the Notch polypeptide is a vertebrate Notch polypeptide. In some embodiments, the vertebrate is a mammal. In some embodiments, the mammal is a human. In some embodiments, the Notch polypeptide is a human Notch 1, human Notch 2, human Notch 3, or human Notch 4 polypeptide. In certain embodiments, the cell or tissue is obtained from a subject, and diagnosing a cell differentiation-associated and/or cell maintenance-associated disease or condition in the cell or tissue is diagnostic for the cell differentiation-associated and/or cell maintenance-associated disease or condition in the subject. In some embodiments, the cell differentiation-associated and/or cell maintenance-associated disease or condition is cancer, a neurodegenerative disease, development, or cell and tissue repair. In some embodiments, the cell differentiation-associated and/or cell maintenance-associated disease or condition is cerebral autosomal dominant arteriopathy with subcortical infarcts and leukoencephalopathy (CADASIL), Allagile syndrome, leukemia (T-cell acute lymphoblastic), Spondylocostal dystosis, down syndrome, Alzheimer's disease, heart diseases, or a prion disease. In certain embodiments, the subject is an invertebrate. In some embodiments, the subject is a vertebrate. In some embodiments, the vertebrate is a mammal. In some embodiments, the mammal is a human. In certain embodiments, the antibodies or antigen-binding fragments thereof are detectably labeled. In some embodiments, the detectable label is a fluorescent, enzyme, radioactive, metallic, biotin, chemiluminescent, or bioluminescent label. In some embodiments, the detectable label is a fluorescent label.

According to yet another aspect of the invention, methods of determining a level of Notch signaling in a cell or tissue are provided. The methods include contacting the cell or tissue with one or more antibodies or antigen-binding fragments thereof that specifically bind a first, second, or third domain of a Notch polypeptide, wherein (a) the first domain is an extracellular domain of the Notch polypeptide, (b) the second domain consists of a Ram23+Ankyrin domain of the Notch polypeptide, and (c) the third domain consists of a C-terminal domain of the Notch polypeptide; detecting the level of specific binding of the one or more antibodies or antigen-binding fragments thereof to the cell or tissue; and comparing the level of binding of the one or more antibodies or antigen-binding fragments thereof, with a control level of binding of the one or more antibodies or antigen-binding fragments thereof as a determination of the level of Notch signaling in the cell or tissue, wherein (i) a higher level of binding of an antibody or antigen-binding fragment thereof to the first domain in the cell or tissue compared to the control level of binding of the antibody or antigen-binding fragment thereof to the first domain indicates that the cell or tissue has a lower level of Notch signaling than the control level, (ii) a higher level of binding of an antibody or antigen-binding fragment thereof to the second domain in the cell or tissue compared to the control level of binding of the antibody or antigen-binding fragment thereof to the second domain indicates the cell or tissue has a lower level of Notch signaling than the control level, and (iii) a higher level of binding of an antibody or antigen-binding fragment thereof to the third domain in the cell or tissue compared to the control level of binding of the antibody or antigen-binding fragment thereof to the third domain indicates that the cell or tissue has a higher level of Notch signaling than the control level. In some embodiments, the cell or tissue is contacted with two or more antibodies or antigen-binding fragments thereof wherein at least two of the two or more antibodies or antigen-binding fragments thereof specifically bind a different one of the first or second domain and the third domains of the Notch polypeptide. In some embodiments, the cell or tissue is contacted with least one antibody or antigen-binding fragment thereof that specifically binds to the C-terminal domain of a Notch polypeptide and at least one antibody or antigen-binding fragment thereof that specifically binds to the extracellular Notch polypeptide domain or to the Ram23+Ankyrin Notch polypeptide domain. In certain embodiments, the methods also include determining the ratio of specific binding of one or more antibodies or antigen-binding fragments thereof to the extracellular or Ram23+Ankyrin domains to one or more antibodies or antigen-binding fragments thereof to the C-terminal domain of the Notch polypeptide as a measure of the level of Notch signaling in the cell or tissue. In some embodiments, the Notch polypeptide is an invertebrate Notch polypeptide. In some embodiments, the Notch polypeptide is a vertebrate Notch polypeptide. In some embodiments, the vertebrate is a mammal. In certain embodiments, the mammal is a human. In some embodiments, the Notch polypeptide is a human Notch 1, human Notch 2, human Notch 3, or human Notch 4 polypeptide. In some embodiments, diagnosing a cell differentiation-associated and/or cell maintenance-associated disease or condition in a subject includes determining the level of Notch signaling in a cell or tissue sample from the subject using any of the foregoing methods and embodiments, wherein the level of Notch signaling in the cell or tissue sample compared to a control level indicates the presence of the cell differentiation-associated and/or cell maintenance-associated disease or condition in the subject. In some embodiments, the subject is an invertebrate. In certain embodiments, the subject is a vertebrate. In some embodiments, the vertebrate is a mammal. In some embodiments, the mammal is a human. In some embodiments, the cell differentiation-associated and/or cell maintenance-associated disease or condition is cancer, a neurodegenerative disease, development, or cell and tissue repair. In certain embodiments, the cell differentiation-associated and/or cell maintenance-associated disease or condition is cerebral autosomal dominant arteriopathy with subcortical infarcts and leukoencephalopathy (CADASIL), Allagile syndrome, leukemia (T-cell acute lymphoblastic), Spondylocostal dystosis, down syndrome, Alzheimer's disease, heart diseases, or a prion disease. In some embodiments, the cell differentiation-associated and/or cell maintenance-associated disease or condition is cerebral autosomal dominant arteriopathy with subcortical infarcts and leukoencephalopathy (CADASIL) and the Notch polypeptide is human Notch 3 polypeptide.

In some embodiments, the method includes selecting a treatment for a subject with a cell differentiation-associated and/or cell maintenance-associated disease or condition comprising, determining the level of Notch signaling in a cell or tissue of the subject using any embodiments of the foregoing method.

According to yet another aspect of the invention, methods for identifying a change in the level of Notch signaling in a subject are provided. The methods include determining in a first biological sample obtained from the subject an amount of truncated Notch polypeptide, determining in a second biological sample obtained from the subject at a time later than the first biological sample an amount of truncated Notch polypeptide, comparing the level of truncated Notch polypeptide in the first and second samples, wherein a difference in the level of truncated Notch polypeptide in the first sample compared to the level of truncated Notch polypeptide in the second sample identifies a change in the level of Notch signaling in the subject. In some embodiments, determining the amount of truncated Notch polypeptide comprises the use of immunodetection methods. In certain embodiments, the level of truncated Notch polypeptide is determined by contacting the biological sample with one or more antibodies or antigen-binding fragments thereof that specifically bind to one or more domain(s) present in a truncated Notch polypeptide and one or more antibodies or antigen-binding fragments thereof that specifically bind to the C-terminal domain of a Notch polypeptide, detecting the level of binding of the antibodies or antigen-binding fragments thereof and comparing the level of binding of the antibodies or antigen-binding fragments thereof that specifically the domain(s) present in a truncated Notch polypeptide to the level of binding of the antibodies or antigen-binding fragments thereof that specifically bind to the C-terminal domain of the Notch polypeptide as a measure of the level of truncated Notch polypeptide in the biological sample. In some embodiments, a higher level of truncated Notch polypeptide in the first biological sample compared to the second biological sample identifies a higher level of Notch signaling in the second biological sample than in the first biological sample. In some embodiments, a lower level of truncated Notch polypeptide in the first biological sample compared to the second biological sample identifies a lower level of Notch signaling in the second biological sample than in the first biological sample. In some embodiments, the cell or tissue is contacted with least one antibody or antigen-binding fragment thereof that specifically binds to the C-terminal domain of a Notch polypeptide and at least one antibody or antigen-binding fragment thereof that specifically binds to the extracellular Notch polypeptide domain or to the Ram23+Ankyrin Notch polypeptide domain. In certain embodiments, the Notch polypeptide is an invertebrate Notch polypeptide. In some embodiments, the Notch polypeptide is a vertebrate Notch polypeptide. In some embodiments, the Notch polypeptide is a mammalian Notch polypeptide. In some embodiments, the Notch polypeptide is a human Notch polypeptide. In certain embodiments, the Notch polypeptide is a human Notch 1, human Notch 2, human Notch 3, or human Notch 4 polypeptide. In some embodiments, the subject is an invertebrate. In some embodiments, the subject is a vertebrate. In some embodiments, the vertebrate is a mammal. In certain embodiments, the mammal is a human. In some embodiments, the antibodies or antigen-binding fragments thereof are detectably labeled. In some embodiments, the detectable label is a fluorescent, enzyme, radioactive, metallic, biotin, chemiluminescent, or bioluminescent label. In some embodiments, the detectable label is a fluorescent label.

According to another aspect of the invention, methods of identifying modulation of Notch signaling by a candidate pharmacological agent, are provided. The methods include contacting a test cell or tissue with a candidate pharmacological agent, determining an amount of truncated Notch polypeptide of the test cell or tissue, comparing the amount of truncated Notch polypeptide of the test cell or tissue to an amount of truncated Notch polypeptide of a control cell or tissue, wherein a relative increase or relative decrease in the amount of truncated Notch polypeptide in the test cell or tissue compared to the control cell or tissue identifies the candidate pharmacological agent as modulating Notch signaling. In certain embodiments, a relative increase in the amount of truncated Notch polypeptide in the test cell or tissue compared to the amount of truncated Notch polypeptide of the control cell or tissue identifies the candidate pharmacological agent as decreasing Notch signaling in the cell or tissue. In some embodiments, a relative decrease in the amount of truncated Notch polypeptide in the test cell or tissue compared to the amount of truncated Notch polypeptide of the control cell or tissue identifies the candidate pharmacological agent as increasing Notch signaling in the cell or tissue. In some embodiments, determining the amount of truncated Notch polypeptide comprises the use of immunodetection methods. In some embodiments, the amount of truncated Notch polypeptide is determined by contacting the cell or tissue with one or more antibodies or antigen-binding fragments thereof that specifically bind to one or more domain(s) present in a truncated Notch polypeptide and one or more antibodies or antigen-binding fragments thereof that specifically bind to the C-terminal domain of a Notch polypeptide, detecting the level of binding of the antibodies or antigen-binding fragments thereof to the cell or tissue, and comparing the level of binding of the antibodies or antigen-binding fragments thereof that specifically bind the domain(s) present in a truncated Notch polypeptide to the binding of the antibodies or antigen-binding fragments thereof that specifically bind to the C-terminal domain of the Notch polypeptide as a determination of the amount of truncated Notch polypeptide of the cell or tissue. In certain embodiments, the cell or tissue is contacted with least one antibody or antigen-binding fragment thereof that specifically binds to the C-terminal domain of a Notch polypeptide and at least one antibody or antigen-binding fragment thereof that specifically binds to the extracellular Notch polypeptide domain or to the Ram23+Ankyrin Notch polypeptide domain. In some embodiments, the one or more antibodies or antigen-binding fragments thereof that specifically bind to truncated Notch polypeptide are antibodies or antigen-binding fragments thereof that specifically bind either the extracellular domain of the Notch polypeptide or a Ram23+Ankyrin domain of the Notch polypeptide. In some embodiments, the truncated Notch polypeptide is a Notch polypeptide without an extracellular Notch polypeptide domain and without a C-terminal Notch polypeptide domain. In some embodiments, the truncated Notch polypeptide is a Notch polypeptide without a transcription activating domain (TAD). In certain embodiments, the truncated Notch polypeptide is a Notch polypeptide without a Ram23+Ankyrin Notch polypeptide domain and without a C-terminal Notch polypeptide domain. In some embodiments, the truncated Notch polypeptide consists of a Notch polypeptide extracellular domain. In some embodiments, the cell or tissue is an invertebrate cell or tissue. In some embodiments, the cell or tissue is a vertebrate cell or tissue. In certain embodiments, the Notch polypeptide is an invertebrate Notch polypeptide. In some embodiments, the Notch polypeptide is a vertebrate Notch polypeptide. In some embodiments, the vertebrate is a mammal. In certain embodiments, the mammal is a human. In some embodiments, the Notch polypeptide is a human a, human Notch 2, human Notch 3, or human Notch 4 polypeptide. In some embodiments, the antibodies or antigen-binding fragments thereof are detectably labeled. In some embodiments, the detectable label is a fluorescent, enzyme, radioactive, metallic, biotin, chemiluminescent, or bioluminescent label.

According to yet another aspect of the invention, methods of identifying an effect of a candidate pharmacological agent on Notch signaling are provided. The methods include contacting a test cell or tissue with a candidate pharmacological agent, determining a ratio of an amount of truncated Notch polypeptide of the cell or tissue to an amount of full-length Notch polypeptide of the cell or tissue, comparing the ratio of the amount of truncated Notch polypeptide to the amount of full-length polypeptide of the cell or tissue to a ratio of the amount of truncated Notch polypeptide to the amount of full-length Notch polypeptide of a control cell or tissue, wherein a wherein a relative increase or relative decrease in the ratio of the amount of truncated Notch polypeptide to the amount of full-length polypeptide of the cell or tissue in the test cell or tissue compared to the control identifies an effect of the candidate pharmacological agent on Notch signaling. In certain embodiments, determining the ratio of the amount of truncated Notch polypeptide of the cell or tissue to the amount of full-length Notch polypeptide of the cell or tissue comprises the use of immunodetection methods. In some embodiments, a relative increase in the amount of truncated Notch polypeptide in the test cell sample compared to the control indicates a decrease in Notch signaling by the candidate pharmacological agent. In some embodiments, a relative decrease in the amount of truncated Notch polypeptide in the test cell sample compared to the control indicates an increase in Notch signaling by the candidate pharmacological agent. In some embodiments, the truncated Notch polypeptide is a Notch polypeptide without an extracellular Notch polypeptide domain and without a C-terminal Notch polypeptide domain. In certain embodiments, the truncated Notch polypeptide is a Notch polypeptide without a transcription activating domain (TAD). In some embodiments, the truncated Notch polypeptide is a Notch polypeptide without a Ram23+Ankyrin Notch polypeptide domain and without a C-terminal Notch polypeptide domain. In some embodiments, the truncated Notch polypeptide consists of a Notch polypeptide extracellular domain. In some embodiments, determining the ratio of the amount of truncated Notch polypeptide of the cell or tissue to the amount of full-length Notch polypeptides of the cell or tissue includes contacting the cell with one or more antibodies or antigen-binding fragments thereof that specifically bind one or more domain(s) of the truncated Notch polypeptide, contacting the cell or tissue with one or more antibodies or antigen-binding fragments thereof that specifically bind the C-terminal domain of the Notch polypeptide; detecting the level of specific binding of the domain(s) present in a truncated Notch polypeptide and Notch polypeptide C-terminal antibodies or antigen-binding fragments thereof in the cell or tissue, and comparing the level of specific binding of the domain(s) present in a truncated Notch polypeptide and Notch polypeptide C-terminal antibodies or antigen-binding fragments thereof to detect the ratio of truncated and full-length Notch polypeptide in the cell or tissue. In certain embodiments, the cell or tissue is contacted with least one antibody or antigen-binding fragment thereof that specifically binds to the C-terminal domain of a Notch polypeptide and at least one antibody or antigen-binding fragment thereof that specifically binds to the extracellular Notch polypeptide domain or to the Ram23+Ankyrin Notch polypeptide domain. In some embodiments, the cell is an invertebrate cell. In some embodiments, the cell is a vertebrate cell. In some embodiments, the Notch polypeptide is an invertebrate Notch polypeptide. In some embodiments, the Notch polypeptide is a vertebrate Notch polypeptide. In certain embodiments, the vertebrate is a mammal. In some embodiments, the mammal is a human. In some embodiments, the Notch polypeptide is human Notch 1, human Notch 2, human Notch 3, or human Notch 4 polypeptide. In some embodiments, the antibodies or antigen-binding fragments thereof are detectably labeled. In certain embodiments, the detectable label is a fluorescent, enzyme, radioactive, metallic, biotin, chemiluminescent, or bioluminescent label. In some embodiments, the detectable label is a fluorescent label.

According to yet another aspect of the invention, methods for preparing a cell, tissue, or non-human animal model of a disorder characterized by altered Notch signaling are provided. The methods include increasing or decreasing the level of truncated Notch polypeptide in a cell, tissue or a non-human animal, to prepare a cell, tissue, or non-human animal model of the disorder. In some embodiments, the increasing or decreasing the level of truncated Notch polypeptide comprises a genetic, chemical, pharmaceutical means. In some embodiments, the method also includes detecting in the cell, tissue or non-human animal symptoms of a disorder characterized by altered Notch signaling. In certain embodiments, the level of truncated Notch polypeptide is increased in the cell, tissue, or animal and the disorder is characterized by reduced Notch signaling. In some embodiments, the level of truncated Notch polypeptide is decreased in the cell, tissue, or animal and the disorder is characterized by increased Notch signaling. In some embodiments, the cell, tissue, or animal model is a model for a cell differentiation-associated and/or cell maintenance-associated disease or condition. In certain embodiments, the cell differentiation-associated and/or cell maintenance-associated disease or condition is cancer, a neurodegenerative disease, development, or cell and tissue repair. In some embodiments, the cell differentiation-associated and/or cell maintenance-associated disease or condition is cerebral autosomal dominant arteriopathy with subcortical infarcts and leukoencephalopathy (CADASIL), Allagile syndrome, leukemia (T-cell acute lymphoblastic), Spondylocostal dystosis, down syndrome, Alzheimer's disease, heart diseases, or a prion disease.

### BRIEF DESCRIPTIONS OF THE DRAWINGS

FIG. 1 is a diagram showing the structure of N and the epitope regions of N antibodies. FIG. 1A shows the structure of the full-length N molecule (NFull) and the major components of SuH/N^{intra} signaling. See text in Introduction for meaning of abbreviated terms. FIG. 1B. Epitope regions of the various antibodies used in the study. Filled bars=epitope regions determined, confirmed, or refined by us using in vivo immuno-cytochemistry and immuno-fluorescence procedures and ex vivo immuno-precipitation and western blot procedures, with materials obtained from flies, S2 cells, and bacteria expressing N fragments; unfilled bars=epitope regions that are published or determined by others.

FIG. 2 shows digitized images of gels and diagrams of results demonstrating forms of N in wild type yw embryos that were identified by immunoprecipitation and western blotting procedures. FIG. 2A. Western blots showing N molecules immunoprecipitated by an amino terminus antibody and probed with antibodies made against different regions along the length of the NFull protein. IP Ab=antibody used in immuno-precipitation; WB Ab=antibodies used on western blots. FIG. 2B. Inference of the structures of the different N intracellular domain fragments based on a systematic study of N fragments obtained with all possible immuno-precipitation/western blotting combinations of the N intracellular domain antibodies used in the study (except αNPCR). Positions of possible proteolytic cleavage sites are shown at the bottom. S1=previously described Furin cleavage site; S4-6=newly proposed sites. FIG. 2C. A sample of two western blots showing the different N intracellular fragments immunoprecipitated from embryonic extracts that are described in B. IP Ab=immunoprecipitating antibody; WB Ab=western blotting antibody; P=immuno-precipitate; F=flow through; pre-is =pre-immune serum; [IP Ab]=antibody cleared by precipitation. Lanes 1 and 3 and lanes 5 and 7 represent P and F fractions from the same sample.

FIG. 3 is a diagram showing the structure of notch receptors and relevant features. EGFs=epidermal growth factor-like repeats; TM=transmembrane; UBi=ubiquitination site; TAD=transcription activation site.

FIG. 4 is a diagram illustrating the mechanism of notch signaling. PM=plasma membrane.

FIG. 5 is a diagram providing a sample of CADASIL in human Notch 3. FIG. 5 A shows the structure of Notch 3. FIG. 5B shows CADASIL mutations on the genes encoding Notch 3. (From Joutel, A, K., et al., Lancet 350:1511-1515(1997). Stars: do not involve cysteines; FS=frame shift.

FIG. 6 graph showing evolutionary conservation of Notch extracellular regions in human Notch 1, rat Notch, frog Notch 1, and Drosophila Notch. AVG=average conservation; muts=site of mutations in Drosophila.

FIG. 7 shows a diagram of Notch intracellular domain molecules in Drosophila embryos. S4, S5, and S6=predicted sites.

FIG. 8 is a diagram showing the putative molecule basis for the strong signals obtained with the different Notch antibodies in Drosophila.

FIG. 9 is a schematic diagram showing auto positive and dominant negative regulation of Notch signaling.

FIG. 10 is a schematic diagram showing differentiation of the CNS and the cuticle in Drosophila embryos. NPC=neuronal precursor cells; EPC=epidermal precursor cells.

FIG. 11 is a schematic diagram showing the AFM procedure used to study Notch and DSL interaction and Notch signaling.

FIG. 12 is a histogram showing binding strength (detachment force) between different Notch molecules and Delta.

FIG. 13 is a graph showing the rate of loss of adhesion force between Notch receptors and delta. Line a=S2-N; line b=S2-N¹⁻²¹⁵⁵; line c=S2-N^{nd3}; line d=S2-Nmf; and line e=S2-NΔ1-18.

FIG. 14 shows two graphs showing the loss of adhesion between Notch and Delta is blocked by a Presenillin (Psn) inhibitor. FIG. 14A shows adhesion with no added Psn inhibitor and FIG. 14B shows adhesion with added Psn inhibitor. In FIG. 14A line a=S2-N; line b=S2-N¹⁻²¹⁵⁵; line c=S2-N^{nd3}; line d=S2-Nmf; and line e=S2-NΔB. In FIG. 14B, line a=S2-N; line b=S2-N¹⁻²¹⁵⁵; line c=S2-N^{nd3}; line d=S2-Nmf; line e=S2-NΔB (1× Psn inhibitor); line f=S2-NΔB (5× Psn inhibitor).

FIG. 15 is a schematic diagram showing human notch regions comparable to Drosophila Notch epitope regions.

### DETAILED DESCRIPTION OF THE INVENTION

It has now been discovered that the level of Notch signaling in cells, tissues, and subjects can be identified by determining the amount of truncated Notch polypeptides and/or by determining the ratio of truncated Notch polypeptides to full-length Notch polypeptides.

Examination of Drosophila Notch, which works very similarly to the mammalian Notch, have now shown that the Notch receptor polypeptide is cleaved to produce dominant negative molecules that are part of an auto-down-regulatory mechanism. It has been determined that the ratio of the level of these truncated Notch polypeptides to that of the full-length Notch molecule is an accurate indicator of the level of Notch signaling during differentiation. In some embodiments, a cocktail of different antibodies made against some specific regions of the Notch polypeptide and calibrated to give colored (fluorescent) readouts can be used as an indicator of the level of Notch signaling in cells and tissues. Comparison between the wild type and test cells or tissues may be used to indicate whether Notch signaling is normal or abnormal. These methods can be used for in diagnostic methods for clinical application and are also useful as research tools to study the Notch signaling pathway and its role in development, differentiation, and/or maintenance of cells. The invention includes, in part, reliable, predictive, and generally applicable assays and methods to determine the level of Notch signaling in vivo in the course of normal tissue differentiation, normal organ development, and abnormal or disease development.

The invention relates, in part, to determining the level of truncated and full-length Notch polypeptides of a cell, tissue, or subject. Notch polypeptides are expressed in vertebrate and invertebrate organisms and much work on Notch has been performed in Drosophila, as it serves as a model organism for Notch signaling. The mammalian and human Notch receptors function very similarly to the Drosophila Notch receptor. The amino acid sequence of wild-type full-length Drosophila Notch polypeptide is the translated mRNA sequence of Genbank Acc. No. M13689, K03507, db_xref=″Gadfly:AE003426.2, with an amino acid sequence of Genbank Acc. No. AAF45848.2).

There are at least four identified human wild-type Notch polypeptides: Human Notch 1, is encoded by the mRNA sequence set forth as Genbank Acc. No. NM_{—}017617, and has the amino acid sequence set forth as Genbank Acc. No. NP_{—}060087; Human Notch 2, which is encoded by the mRNA sequence set forth as Genbank Acc. No. NM_{—}024408, and has the amino acid sequence set forth as Genbank Acc. No. NP_{—}077719; Human Notch 3, which is encoded by the mRNA sequence of Genbank Acc. No. NM_{—}000453, and has the amino acid sequence of Genbank Acc. No. NP_{—}000426; and Human Notch 4, which is encoded by the mRNA sequence of Genbank Acc. No. NM_{—}004557, and has the amino acid sequence of Genbank Acc. No. NP_{—}004548. It will be understood that a Notch polypeptide may include one or more mutations and/or alterations in its nucleic acid or amino acid sequence compared the wild-type sequences provided herein. The methods of the invention include the determination of activity of Notch signaling of wild-type as well as various allelic variants and mutated Notch polypeptides that differ from a wild-type sequence.

The methods of the invention include the detection of amounts and ratios of truncated Notch polypeptides. As used herein, the term “Notch polypeptide” means full-length as well as truncated Notch polypeptides. A full-length Notch polypeptide includes three domains. A first Notch polypeptide domain is the extracellular domain and includes the amino acid residues that form the extracellular portion of the Notch receptor. A second Notch polypeptide domain is referred to herein as the Ram23+Ankyrin domain (also referred to herein as the Ram 23+Ankyrin repeat region) and includes the amino acid residues from the intracellular end of the transmembrane domain to the end of the ankyrin repeats in the Notch polypeptide. The third Notch polypeptide domain is referred to herein as the C-terminal Notch polypeptide domain. This domain includes the amino acid residues beginning with the residue that immediately following the Ram23+Ankyrin domain and includes the residues through the C-terminal end of the Notch polypeptide. It will be recognized by those of skill in the art that for each of the human Notch polypeptides, the number of amino acid residues in each domain may differ and conservative substitutions and deletions may be introduced, but the domains can be readily identified by their structural features as described above. Amino acid residues comprising these domains in Drosophila and human notch polypeptides are shown in the table 1 below.

TABLE 1
Comparable Drosophila and Human Notch
regions (in amino acid numbers)

| | Extracellular | Ram 23 + Ankyrin | C-terminal |
|---|---|---|---|
| | domain | repeat domain | domain |
| Drosophila Notch | 1-1750 | 1765-2155 | 2155-2703 |
| Human Notch 1 | 1-1735 | 1757-2130 | 2130-2556 |
| Human Notch 2 | 1-1677 | 1700-2075 | 2075-2471 |
| Human Notch 3 | 1-1648 | 1670-2040 | 2040-2318 |
| Human Notch 4 | 1-1440 | 1465-1790 | 1790-1964 |

The methods of the invention include, in part, the determination of the amount of truncated Notch polypeptides in cells and tissues. Two types of truncated Notch polypeptides are (1) the truncated polypeptide that includes the extracellular domain, but does not have either the Ram23+Anks domain or the C-terminal domain and (2) a truncated Notch polypeptide that includes the Ram 23/Ankyrin domain but does not have either the extracellular domain or the C-terminal domain. The methods of the invention may include determining the amount of each of two different truncated Notch polypeptides. A full-length Notch polypeptide will include the C-terminal domain (along with the remainder of the Notch polypeptide), whereas neither of the described truncated Notch polypeptides include the C-terminal domain.

The methods of the invention can be used to determine the level of Notch signaling in a cell, tissue, or subject and may be used to diagnose cell differentiation- and maintenance-associated diseases or conditions in a cell, tissue, or subject. The methods of the invention involve determining the amount of truncated Notch polypeptides in a cell or tissue as a measure of the amount of Notch signaling in the cell or tissue. The methods are therefore useful to detect a difference in the level of Notch signaling in a cell or tissue compared to a control level of Notch signaling. As used herein, the term “cell differentiation-associated and/or cell maintenance-associated disease or condition” means a condition or disease in which cell differentiation and/or cell maintenance occurs. Embryonic development is an example of a cell differentiation-associated condition because embryonic development is associated with differentiation of cells and tissues. For example, the determination of cell fate, lineage, are events that occur in development that are associated with the differentiation and/or maintenance of cells. It will be clear to those of skill in the art, that not all cell differentiation- and maintenance-associated conditions are abnormal or are indicative of illness. Some differentiation- and maintenance-associated conditions represent a normal state of a cell or tissue in development, growth, healing, and day-to-day cellular operations. In other embodiments, a cell differentiation- and/or maintenance-associated disease or condition may be an illness, injury, or other abnormal indication in a cell. In each case, the disease or condition is associated with Notch signaling. Examples of cell differentiation- and/or maintenance-associated diseases and conditions e.g. includes, but are not limited to neurodegenerative diseases (e.g. Parkinson's disease (PD), Alzheimer's disease, etc.), normal cell and tissue development, normal cell and tissue aging, stroke, cardiovascular disease, macular degeneration, effects of toxin exposure, CNS diseases, metabolic disorders, infections, cell and tissue repair, cerebral autosomal dominant arteriopathy with subcortical infarcts and leukoencephalopathy (CADASIL), cancer, Allagile syndrome, leukemia (T-cell acute lymphoblastic), Spondylocostal dystosis, down syndrome, heart disease, and prion disease, etc. In each disease and condition, an alteration in Notch signaling is associated with the state of the cell or tissue.

The assays described herein are carried out on samples. In some embodiments, a sample is a biological sample obtained from a subject. In some embodiments, a sample can be synthetic or (e.g. laboratory prepared) and not obtained from a subject. As used herein, the term “subject” includes vertebrate and invertebrate organisms. Examples of invertebrate organisms include, but are not limited to, drosophila, nematodes, etc. Vertebrate subjects may include fish, birds, and mammals. Subjects include but are not limited to: humans, non-human primates, cats, dogs, sheep, pigs, horses, cows, rodents such as mice, rats, hamsters, gerbils, etc. In some aspects of the invention, a subject is known to have, or is considered to be at risk of having, a disease or condition associated with abnormal Notch signaling—e.g. a cell differentiation- and/or maintenance-associated disease or condition. In some embodiments, a subject is a mammal that is an animal model for a cell differentiation- and/or maintenance-associated disease or condition. One of ordinary skill in the art will recognize that animal models of a cell differentiation- and/or maintenance-associated disease or condition (e.g. see Examples) may be generated by genetic engineering or by chemical or physical treatment to alter the level of truncated Notch polypeptide and to alter the level of Notch signaling in the animal.

As used herein, a “biological sample” encompasses a variety of sample types obtained from an individual through invasive or non-invasive approaches (e.g., urine collection, blood drawing, needle aspiration, and other procedures). The definition also includes samples that have been manipulated in any way after their procurement (through invasive or non-invasive approaches), such as by treatment with reagents, solubilization, or enrichment for certain components, such as proteins or polynucleotides. The term “biological sample” includes, but is not limited to, any body tissue or body fluid sample obtained from a subject. Body fluids include: urine, blood, saliva, lacrimal fluid, synovial fluid, cerebrospinal fluid, sweat, pulmonary secretions (sputum), seminal fluid, and feces. Preferred are body fluids, for example, lymph, saliva, blood, urine, and the like. Body tissues may be from skin, nerve, CNS tissue, tumor tissue, etc. A biological sample may be cells or tissue in and obtained from culture as well as cells or tissues in or obtained from a subject. Examples of cells or tissues in culture that may be used in the methods of the invention as test cells tissues or as control cells or tissues include cells or tissues known to be afflicted with a cell differentiation- and/or maintenance-associated disease or condition (e.g. AD, CADASIL, or Parkinson's disease, etc). In some embodiments, the cells (e.g. cultured cells) may not be afflicted with a cell differentiation- and/or maintenance-associated disease or condition and may serve as control cells or tissues. Cells and tissues that are free of a specific cell differentiation- and/or maintenance-associated disease or condition may be examined in parallel with a test cell or tissue and may serve as a control cell or tissue. Such control cells and tissues may be useful to determine a “normal” level of Notch signaling.

In some embodiments the amount of truncated and/or full-length Notch polypeptides may be determined in a cell and/or tissue that is in vivo, e.g., in a subject. The invention provides a method for detecting the level or amount of a Notch polypeptide (and Notch signaling) in cells and/or tissue in vivo. The methods include, in part, administering to a subject antibodies that selectively bind a domain of a truncated Notch polypeptide and/or antibodies that bind to full-length Notch polypeptide that is conjugated to a detectable label, exposing the subject to a means for detecting the detectable marker in the cells and/or tissues of the subject—e.g. via NMR, confocal microscopy, tomography, etc, and determining the level of Notch polypeptide or ratio of truncated to full-length notch polypeptide.

According to some aspects of the invention, agents that bind specifically to full-length and truncated Notch polypeptides can be prepared and used to identify and quantitate the amount of truncated Notch polypeptide in a sample and the ratio of the amount of trunctated Notch polypeptide to the amount of full-length Notch polypeptide in the sample. As used herein, “binding specifically to” or “specifically binds” mean capable of distinguishing the identified material from other materials sufficient for the purpose to which the invention relates. For example, “specifically binds” the extracellular domain of a Notch polypeptide, mean that the agent has the ability to bind to and distinguish the extracellular domain of a Notch polypeptide from other polypeptides, proteins or domains. An antibody that specifically binds to a Notch polypeptide may preferentially bind to the Notch polypeptide with an affinity that is at least two-fold, 50-fold, 100-fold, or greater than its affinity for binding to a non-specific antigen (e.g. BSA, casein) other than the Notch polypeptide or domain.

In some embodiments, antibodies or antigen-binding fragments thereof that specifically bind to a full-length or truncated Notch polypeptides can be used to assess the presence of polypeptides that include the extracellular domain, the Ram23+Ankyrin domain, or the C-terminal domain of a Notch polypeptide in a sample. For example, an antibody or antigen-binding fragment thereof that specifically binds the extracellular domain of a Notch polypeptide, will bind to either full-length Notch polypeptide or to a truncated polypeptide that includes the Ram23+Ankyrin domain. An antibody or antigen-binding fragment thereof that specifically binds the Ram23+Ankyrin domain will bind to either a full-length Notch polypeptide or to a truncated polypeptide that includes the Ram23+Ankyrin domain. An antibody or antigen-binding fragment thereof that specifically binds the C-terminal domain of a Notch polypeptide, will bind only a full-length Notch polypeptide and will not bind one of the truncated Notch polypeptides. Thus, a combination of antibodies or antigen-binding fragments may be used to determine either the amount of truncated plus full-length Notch polypeptide in a cell or tissue sample, or a ratio of the amount of truncated to full-length Notch polypeptide in a cell or tissue sample.

As a non-limiting representation, if contacting a cell with a combination of antibodies that bind to the extracellular domain and to the Ram23 t Ankyrin or to the C-terminal Notch polypeptide domain, results in the determination of “X” as the amount of full-length Notch polypeptide, and X+Y as the amount for the extracellular domain alone, it indicates that at least a level of Y of truncated Notch polypeptide is present in the sample in addition to the “X” amount of the full-length Notch polypeptide. This amount is then compared to the amount of truncated Notch polypeptide in a control sample as a measure of the level of Notch signaling in the cell compared to the control cell. If the amount of truncated Notch polypeptide is determined to be higher in the cell than in a “normal” control cell, then it indicates that the level of Notch signaling in the cell is lower than that of the control cell. If the amount of truncated notch polypeptide is determined to be lower in the cell than in a “normal” control cell, then it indicates that the level of notch signaling in the cell is higher than that of the control cell.

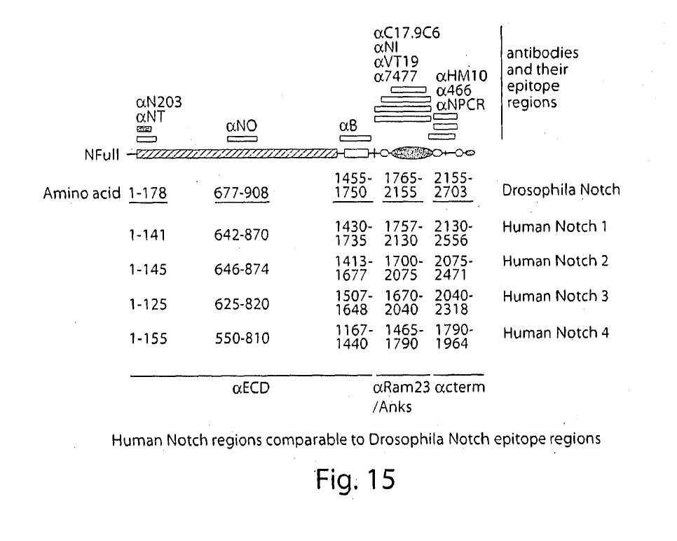

One of ordinary skill in the art will recognize that various combinations of antibodies that bind to the three domains of a Notch polypeptide can be used to determine the amount and relative amounts of truncated Notch polypeptides and full-length Notch polypeptides (see Examples section for additional information). Differences in the amount of binding of the various antibodies to the different domains of Notch polypeptide can thus be used to indicate the presence and/or amount of truncated Notch polypeptide and the level of Notch signaling in a cell or tissue sample. Examples of regions of the Notch polypeptides from Drosophila and Human Notch 1, 2, 3, and 4 are provided in FIG. 15. FIG. 15 illustrates the epitope regions of Drosophila Notch polypeptide against which antibodies have been generated (open and black bars represent antibodies). The antibodies are shown above the corresponding amino acid region of the Notch polypeptide. Similar epitope regions are provided for the human Notch 1-4 polypeptides in FIG. 15.

Methods to determine the level of Notch signaling may include the use of binding polypeptides, such as include antibodies and antigen-binding fragments thereof, to detect levels and/or ratios of Notch polypeptides as described herein. It will be understood by those of skill in the art, that antigen-binding fragments of antibodies useful in the methods of the invention, may also be used in the methods of the invention. An antigen-binding fragment of an antibody is a fragment of the antibody that retains the function of the whole antibody and has the ability to specifically bind to the same antigen target as the antibody.

The antibodies and antigen-binding fragments thereof of the invention can be used for the assay Notch polypeptide levels and amounts using known methods including, but not limited to, immunocytochemistry, flow cytometry, enzyme linked immunosorbent (ELISA) assays, immunoprecipitations, electrophoretic methods, chromotographic methods, and Western blots, etc. Antibodies or antigen-binding fragments thereof may be used to determine levels and amounts of Notch polypeptides using additional standard methods known to those of ordinary skill in the art. Antibodies useful in the methods of the invention may be conjugated to a solid support.

The antibodies of the present invention may be prepared by any of a variety of methods, including administering protein, fragments of protein, cells expressing the protein or fragments thereof and the like to an animal to induce polyclonal antibodies. The production of monoclonal antibodies is according to techniques well known in the art. As detailed herein, such antibodies or antigen-binding fragments thereof may be used for example to identify the presence or level of truncated and/or full-length Notch polypeptides. The antibodies of the invention include monoclonal and polyclonal antibodies.

The antibodies may be coupled to specific detectable labels for detecting and/or imaging of binding to the Notch polypeptide domains. Antibodies may be coupled to specific labeling agents, for example, for imaging of cells and tissues with according to standard coupling procedures. Detectable labels useful in the invention include, but are not limited to: a fluorescent label, an enzyme label, a radioactive label, visual label (e.g. a metallic label such as ferritin or gold), a nuclear magnetic resonance active label, an electron spin resonance label, a positron emission tomography label, a luminescent label, and a chromophore label. Other labeling agents useful in the invention will be apparent to one of ordinary skill in the art. The detectable labels of the invention can be attached to the binding peptides (e.g. antibodies or antigen-binding fragments thereof) by standard protocols known in the art. In some embodiments, the detectable labels may be covalently attached to a binding peptides (e.g. antibodies or antigen-binding fragments thereof) of the invention. The covalent binding can be achieved either by direct condensation of existing side chains or by the incorporation of external bridging molecules. In some embodiments a detectable label may be attached to a binding peptides (e.g. antibodies or antigen-binding fragments thereof) of the invention using genetic methods. In some embodiments of the invention, more than one type of detectable label may be attached to a binding peptides (e.g. antibodies or antigen-binding fragments thereof) for use in the methods of the invention.

Significantly, as is well known in the art, only a small portion of an antibody molecule, the paratope, is involved in the binding of the antibody to its epitope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd Fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205. Thus, for example, PCT International Publication Number WO 92/04381 teaches the production and use of murine RSV antibodies in which at least a portion of the murine FR regions have been replaced by FR regions of human origin. Such antibodies, including fragments of intact antibodies with antigen-binding ability, are often referred to as “chimeric” antibodies. Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)2, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or nonhuman sequences. The present invention also includes so-called single chain antibodies.

Thus, the invention involves polypeptides of numerous size and type that bind specifically to domains of a Notch polypeptide. Thus, in some embodiments, an antibody that specifically binds to the C-terminal domain of a Notch polypeptide will bind full-length Notch polypeptide but will not bind a truncated Notch polypeptide. Antibodies that bind the extracellular domain or the Ram23+Ankyrin domain in conjunction with an antibody that specifically binds a C-terminal domain of a Notch polypeptide can be used to determine relative amounts of truncated and full-length Notch polypeptide in a cell or tissue sample. Thus, using the differential domains of the full-length and truncated Notch polypeptides allows the determination of the presence and/or amount of truncated Notch polypeptide in a sample. One of ordinary skill will recognize that the different domains in the truncated versus full-length Notch polypeptides allow the use of binding peptides (e.g. antibodies) that specifically bind to certain domains to determine the presence and/or amount of truncated Notch polypeptide in a cell or tissue sample.

Binding polypeptides that are useful in the methods of the invention, may be derived also from sources other than antibody technology. For example, such polypeptide binding agents can be provided by degenerate peptide libraries that can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to a domain of a Notch polypeptide. This process can be repeated through several cycles of reselection of phage that bind to a domain of a Notch polypeptide such as the extracellular domain, the Ram23+Ankyrin domain or the C-terminal domain. Repeated rounds lead to enrichment of phage bearing particular sequences. DNA sequences analysis can be conducted to identify the sequences of the expressed polypeptides. The minimal linear portion of the sequence that binds to a domain of Notch polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to a domain of a Notch polypeptide. Thus, amino acid sequences that make up part or all of a Notch polypeptide domain can be used to screen peptide libraries, including phage display libraries, to identify and select peptide binding partners of the domains.

As detailed herein, the foregoing antibodies and other binding molecules may be used for example to identify truncated and full-length Notch polypeptides and can be used to determine the amount of truncated Notch polypeptide in a sample as measure of Notch signaling in the sample.

The invention provides methods and kits for the determination of the amount of Notch signaling in cells and tissues by determining the amount of truncated and full-length Notch polypeptides in a cell or tissue. For example, in some embodiments, the presence and/or level of truncated and full-length Notch polypeptide are determined. The identification of a higher amount of truncated Notch than is present in a control cell or tissue indicates that Notch signaling is reduced in the sample compared to the control level of Notch signaling. In some embodiments, the amount of a truncated or full-length Notch polypeptide in a cell or tissue or sample is quantified. The quantitation of domains of truncated and/or full-length Notch polypeptides in a cell or tissue may provide a determination of the amount of Notch signaling in the cell or tissue sample.

The invention also involves a variety of assays based upon determining amounts of truncated and full-length Notch polypeptide, and the levels of Notch signaling in subjects. The assays may include (1) identifying the presence or absence of a cell differentiation- and/or maintenance-associated disorder or condition in a subject (2) evaluating a candidate pharmacological agent to treat a cell differentiation- and/or maintenance-associated disorder or condition; (3) selecting a treatment for a cell differentiation- and/or maintenance-associated disorder or condition in a subject; and (4) determining onset, progression, or regression of a cell differentiation- and/or maintenance-associated disorder or condition in a subject. Thus, subjects can be characterized, treatment regimens can be monitored, treatments can be selected and diseases can be better understood using the assays of the present invention.

For example, the invention provides in one aspect a method for measuring the amount of truncated Notch polypeptide of Notch signaling in a cell and/or tissue of a subject, which is a direct indicator of the level of the subject's Notch signaling status. The level of Notch signaling can thus be measured due to the negative correlation between the amount of truncated Notch polypeptides and the amount of Notch signaling. The level of truncated Notch polypeptide (and ratio or truncated Notch polypeptide to full-length Notch polypeptide) thus correlates with the presence of a differentiation- and maintenance-associated disease or condition in the subject. Relatively low amounts of truncated Notch polypeptide and/or low ratios of truncated Notch polypeptide to full-length Notch polypeptide reflect more Notch signaling than do relative high amounts of truncated Notch polypeptide and/or high ratios of truncated Notch polypeptide. Notch polypeptides and Notch signaling are involved in numerous cell differentiation and cell maintenance processes.

Alterations in Notch signaling are in some instances indicative of normal cell changes and in other instances are indicative of abnormal cell changes. The comparison of amounts of truncated and/or ratios of truncated Notch polypeptide to full-length polypeptide with control amounts and ratios can be used to correlate a level or ratio of truncated Notch polypeptides with cell differentiation and/or maintenance-associated disorders and conditions including either normal or abnormal conditions. In a subject with CADASIL, the ratio may be determined to be statistically higher than the normal range, e.g. that of a normal control. Thus, the abnormal ratio is diagnostic for the CADASIL condition in the subject. The ratio in the subject with the differentiation- and maintenance-associated disorder (e.g. CADASIL) may have a 10%, 20%, 30%, 40%, 50%, 60%, 70%, 80%, 90%, 100%, 150%, 200%, 250%, 300%, 400%, or higher ratio (including all percentages in between) of truncated Notch polypeptide to full-length Notch polypeptide.

The assays described herein involve measuring levels of truncated Notch polypeptide. Levels of truncated Notch polypeptide can be determined in a number of ways when carrying out the various methods of the invention. In one particularly important measurement, the level of truncated Notch polypeptide is measured in relation to full-length Notch polypeptide. Thus, the measurement is a relative measure, which can be expressed, for example, as a percentage of total Notch polypeptide. Another measurement of the level of truncated Notch polypeptide is a measurement of absolute levels of truncated Notch polypeptide. This could be expressed, for example, in terms of weight per volume of sample, or number of molecules per cell, etc. Another measurement of the amount of truncated Notch polypeptide is a measurement of the change in the amount of truncated Notch polypeptide over time. This may be expressed in an absolute amount or may be expressed in terms of a percentage increase or decrease over time.

Importantly, amounts of truncated Notch polypeptide are advantageously compared to controls according to the invention. The control may be a predetermined value, which can take a variety of forms. It can be a single cut-off value, such as a median or mean. It can be established based upon comparative groups, such as in groups (e.g. of cells, tissues, or subjects) having normal amounts of Notch signaling and groups having abnormal amounts of Notch signaling. Another example of comparative groups would be groups (e.g. of cells, tissues of subjects) having a particular disease, condition or symptoms and groups without the disease, condition or symptoms. Another comparative group would be a group (e.g. of cells or tissues, or subjects) having a family history of a condition and a group without such a family history. The predetermined value can be arranged, for example, where a tested population is divided equally (or unequally) into groups, such as a low-risk group, a medium-risk group and a high-risk group or into quadrants or quintiles, the lowest quadrant or quintile being individuals with the lowest risk or lowest amount of Notch signaling and the highest quadrant or quintile being individuals with the highest risk or highest amounts of Notch signaling. One of ordinary skill in the art will recognize that in some conditions, the lowest quadrant or quintile being individuals with the lowest risk or highest amount of Notch signaling and the highest quadrant or quintile being individuals with the highest risk or lowest amounts of Notch signaling.

The predetermined value, of a course, will depend upon the particular population selected. For example, an apparently healthy population will have a different ‘normal’ range than will a population that is known to have a condition related to Notch signaling, for example a differentiation and maintenance-associated disease or condition. Accordingly, the predetermined value selected may take into account the category in which a cell, tissue, and/or subject falls. Appropriate ranges and categories can be selected with no more than routine experimentation by those of ordinary skill in the art. By abnormally high it is meant high relative to a selected control. Typically the control will be based on apparently healthy normal cell, tissue, and/or subject.

In measuring the relative amount of truncated Notch polypeptide to full-length Notch polypeptide, those of ordinary skill in the art will appreciate that the relative amount may be determined by measuring either the relative amount of truncated Notch polypeptide or the relative amount of full-length Notch polypeptide. In other words, if 90% of a cell's or tissue's Notch polypeptide is truncated Notch polypeptide, then 10% of the cell's or tissue's Notch polypeptide will be full-length Notch polypeptide. Thus, measuring the level of truncated Notch polypeptide may be carried out by measuring the relative amount of full-length Notch polypeptide.

It will also be understood that the controls according to the invention may be, in addition to predetermined values, samples of materials tested in parallel with the experimental materials. Examples include samples from control populations or control samples generated through manufacture to be tested in parallel with the experimental samples.

In some embodiments of the invention, methods provided are used to determine the level of Notch signaling in cells and or tissues from a subject at risk of having a Notch signaling disorder or a cell differentiation- and/or maintenance-associated disease or condition. As used herein, a subject “at risk” is a subject who is considered more likely to develop a disease state or a physiological state than a subject who is not at risk. A subject “at risk” may or may not have detectable symptoms indicative of the disease or physiological condition, and may or may not have displayed detectable disease prior to the treatment methods (e.g., therapeutic intervention) described herein. “At risk” denotes that a subject has one or more so-called risk factors. A subject having one or more of these risk factors has a higher probability of developing one or more disease(s) or physiological condition(s) than a subject without these risk factor(s). These risk factors can include, but are not limited to, history of family members developing one or more diseases (e.g. CADASIL), related conditions, or pathologies, history of previous disease, age, sex, race, diet, presence of precursor disease, genetic (i.e., hereditary) considerations, and environmental exposure. The level of risk can be assessed using standard methods known to those in the art. For example, based on factors such as medical history, family medical history, and current medical condition, a health care professional may assess a percentage chance that a subject will have or will develop a cell differentiation- and/or maintenance-associated disease or condition. For example, a health care professional may determine that a subject who has a family history of CADASIL may have a 20%, 30%, 40%, 50%, 60%, 70% or more chance of developing CADASIL than an individual with no family history of the disorder. Those of skill in the art will recognize that a subject's level of risk for other a cell differentiation- and/or maintenance-associated disease or conditions can also be evaluated using standard methods.

As mentioned above, it is also possible to characterize Notch signaling by monitoring changes in the absolute or relative amounts of truncated Notch polypeptide (or the ratio of truncated to full-length Notch polypeptide) over time. For example, it is expected that changes in the ratio of truncated to full-length Notch polypeptide correlates with changing levels of Notch signaling. Accordingly one can monitor the ratio of truncated to full-length Notch polypeptide over time to determine if Notch signaling in a tissue or subject are changing. Changes in relative or absolute truncated Notch polypeptide of greater than 0.1% may indicate an cell differentiation- and/or maintenance-associated disease or condition. Preferably, the change in truncated Notch polypeptide amount or ratio, which indicates a cell differentiation- and/or maintenance-associated disease or condition, is greater than 0.2%, greater than 0.5%, greater than 1.0%, 2.0%, 3.0%, 4.0%, 5.0%, 7.0%, 10%, 15%, 20%, 25%, 30%, 40%, 50%, or more.

The invention in another aspect provides a diagnostic method to determine the effectiveness of treatments for cell differentiation- and/or maintenance-associated disease or conditions. The “evaluation of treatment” as used herein, means the comparison of a subject's levels of truncated Notch polypeptide or ratio of truncated to full-length Notch polypeptide measured in samples collected from the subject at different sample times, preferably at least one day apart. The preferred time to obtain the second sample from the subject is at least one day after obtaining the first sample, which means the second sample is obtained at any time following the day of the first sample collection. In some embodiments a second sample is obtained preferably at least 1, 2, 3, 4, 5, 6, 7, 8, 9, 10 or more days or weeks after the time of first sample collection.

The comparison of levels of truncated Notch polypeptide or ratio of truncated to full-length Notch polypeptide in two or more samples, taken at different times, or on different days, is a measure of level of the subject's level of Notch signaling over time and allows evaluation of a treatment the cell, tissue, or subject is undergoing to regulate Notch signaling.

As will be appreciated by those of ordinary skill in the art, the evaluation of the treatment also may be based upon an evaluation of the symptoms or clinical end points of the associated disease, such as the complications of CADASIL. Thus, the methods of the invention also provide for determining the onset, progression, and/or regression of a condition that is characterized by levels of truncated Notch polypeptide or ratios of truncated to full-length Notch polypeptide that differ from that of a control level or ratio. In some instances, the subjects, tissues, and or cells to which the methods of the invention are applied are already diagnosed as having a particular condition or disease. In other instances, the measurement will represent the diagnosis of the condition or disease. In some instances, the subjects will already be undergoing drug therapy for regulating a cell-differentiation- and maintenance-associated disorder, while in other instances the subjects will be without present drug therapy for regulating a cell-differentiation- and maintenance-associated disorder.

Also within the scope of the invention are kits that include materials to carry out the methods of the invention and instructions for use. The kits may include antibodies or antigen-binding fragments thereof or other binding peptides and can further contain at least one additional reagent, such as a control sample. Kits of the invention can be prepared for in vitro diagnosis, prognosis and/or monitoring the amount of truncated Notch polypeptide and/or ratio of truncated to full-length Notch polypeptide and determination of the presence of a cell-differentiation- and maintenance-associated disorder or condition. Kits of the invention may include antibodies or antigen-binding fragments thereof or other binding agents that specifically bind an extracellular Notch polypeptide domain, a Ram23+Ankyrin domain, and/or a C-terminal Notch polypeptide domain. The components of the kits can be packaged either in aqueous medium or in lyophilized form. A kit of the invention, in some embodiments, may further comprise a container containing truncated Notch polypeptide and/or a container containing full-length Notch polypeptide. Some or all of the kit components may be frozen.

A kit of the invention may also include control compounds and solutions for testing the binding activity of the antibodies. Such materials may include, buffer, a non-limiting example of which is sodium phosphate buffer, etc. A kit may also include materials and instructions for detectably labeling a binding agent—e.g. an antibody or antigen-binding fragment thereof.

A kit of the invention may comprise a carrier being compartmentalized to receive in close confinement therein one or more container means or series of container means such as test tubes, vials, flasks, bottles, syringes, or the like. A kit of the invention may also include vials, cuvettes, pipet tips, transfer pipets, solutes, sterile and/or distilled water, one or more control samples, (e.g. blank control, test control), printed graphs, tables, figures, or diagrams, which may be used for interpretation and/or analysis of results or for instructional purposes.

A kit of the invention may also include equipment and/or supplies for determining the level of truncated Notch polypeptide and/or a ratio of truncated to full-length Notch polypeptide. For example, a kit may include ELISA assay materials, gel preparation materials (e.g. solutions, agarose, acrylamide, control markers, dyes and/or labels, etc,). A kit may also include materials for chromatographic analysis, e.g. beads, solvents, solutes, control samples etc, columns, etc.

In some embodiments, materials for analysis of the level of truncated Notch polypeptide and/or a ratio of truncated to full-length Notch polypeptide are provided in a ready-to-use format. In other embodiments, the kits provide materials that can be utilized for determining the level of truncated Notch polypeptide and/or a ratio of truncated to full-length Notch polypeptide in a sample and will be assembled for use by the operator. Some kits of the invention will include all materials necessary for determining the level of truncated Notch polypeptide and/or a ratio of truncated to fall-length Notch polypeptide in a sample, and other kits of the invention will include some, but not all of the materials for the determination of the level of truncated Notch polypeptide and/or a ratio of truncated to full-length Notch polypeptide in a sample. In the later case, additional materials will be provided by the operator and may include: pipets, tubes, gel apparatus, flasks, solutions, enzymes, Notch polypeptides, etc.

### EXAMPLES

### Example 1

### Introduction

Notch (N) is a cell surface protein that is required for differentiation of almost all tissues in animals. Its actions specify two cell types from a population of equipotent cells or establish boundaries between populations of two different cell types. The mechanism of N signaling is as follows. When a ligand such as Delta (D1) expressed on one cell binds N expressed on the neighboring cell, N is proteolytically cleaved, first by the Kuzbanian or TACE metalloproteases (called the S2 cleavage) and subsequently by the Presenilin (Psn)/-γ-secretase complex (called the S3 cleavage). The Notch intracellular domain (N^{intra}) is released from the plasma membrane, translocated to the nucleus, and in association with the transcription factor Suppressor of Hairless (SuH) activates transcription of target genes such as the Enhancer of split Complex (E(spl)C) genes. We refer to this signaling as the SuH/N^{intra} signaling. Cells that initially generate high rates or levels of SuH/N^{intra} signaling, augment this rate or level and become specified as one cell type; cells that initially generate low rates or levels, suppress SuH/N^{intra} signaling completely and become specified as the alternate cell type (Heitzler, P. & Simpson, P. 1991. Cell 64: 1083-1092; Artavanis-Tsakonas, S. et al., 1999. Science 284, 770-776; Mumm, J. S. & Kopan, R. 2000. Dev. Biol. 228, 151-165; Brou, C. et al., 2000. Mol. Cell, 5: 207-216; Lieber, T. et al., 2002. Genes Dev. 16, 209-221; Schweisguth, F. 2004. Curr. Biol. 14: R129-138; Ahiimou, F. et al. 2004. J. Cell Bio.: 167: 1217-1229). This process, often referred to as the lateral inhibition process, is repeatedly used during development for differentiation of various tissues with variations or changes in target genes.

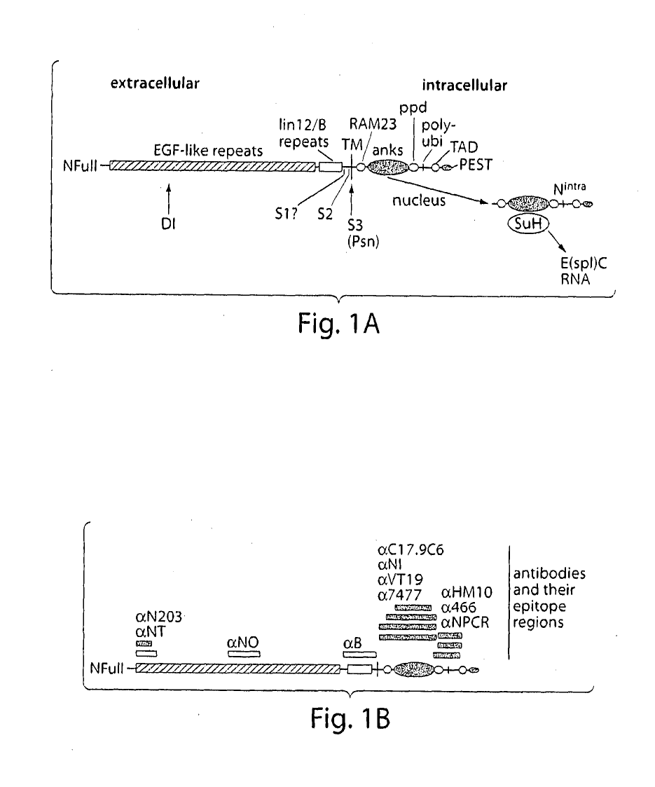

The structural features of N and other components important for SuH/N^{intra} signaling are shown in FIG. 1A. The N protein is composed of the following, in order from the amino terminus (extracellular) to the carboxyl terminus (intracellular): 36 tandem Epidermal Growth Factor-like repeats (EGF-like repeats) which includes the D1 binding site; three cysteine rich repeats called the lin12/B repeats; a potential Furin mediated S1 cleavage site (see below); the S2 cleavage site; the transmembrane domain (TM) within which lies the S3 cleavage site; the Ram 23 region, the ankyrin repeats (anks), and the potential phosphorylation domain (PPD) which are involved in binding SuH; a polyubiquitination site (ubi) implicated in endocytosis; a transcription activation domain (TAD); and a PEST sequence implicated in protein turn over (Wharton, K. A. et al., 1985. Cell, 43: 567-581; Kidd, S. et al., 1986. Mol. Cell. Biol., 6: 3094-3108; Rechsteiner, M. 1988. Adv. Enzyme Regul., 27: 135-151; Fehon, R. G. et al., 1990. Cell 61, 523-534; Rebay, I. et al., 1991. Cell, 67: 687-699; Lieber, T. et al., 1992. Neuron 9, 847-859; Tamura, K. et al., 1995. Curr. Biol. 5, 1416-1423; Matsuno, K. et al., 1997. Development 124, 4265-4273; Logeat F. et al., 1998. Proc. Natl. Acad. Sci. USA. 95: 8108-12; Schroeter, E. H. et al., 1998. Nature, 393:382-6; Kidd, S. et al., 1998. Genes Dev. 12, 3728-40; Kurooka, H. et al., 1998. Nucleic Acids Res. 26, 5448-5455; Brou, C. et al., 2000. Mol. Cell, 5: 207-216; Struhl, G. & Adachi, A. 2000. Mol. Cell, 6: 625-636; Lieber, T. et al., 2002. Genes Dev. 16, 209-221; Le Gall, M. & Giniger, E. 2004. J. Biol. Chem. 279, 29418-29426; Wilkin M. B. et al., 2004. Curr Biol., 14:2237-44; Sakata, T. et al., 2004. Curr Biol., 14: 2228-36).

N receptors at the surfaces of mammalian cells are predominantly the non-covalently linked hetero-dimeric forms of the extracellular and the intracellular domains generated by Furin cleavage at the S1 site (Logeat F. et al., 1998. Proc. Natl. Acad. Sci. USA. 95: 8108-12). N receptors at the surfaces of Drosophila cells appear to be predominantly the covalently linked (collinear) full-length form (Kidd, S. & Lieber, T. 2002. Mech. Dev., 115: 41-51). The reason for this difference is not understood but might be related to the role of N and D1 binding strength in the regulation of the rate of SuH/N^{intra} signaling (Ahimou, F. et al. 2004. J. Cell Bio.: 167: 1217-1229).

One of the better-understood instances of lateral inhibition is the differentiation of the central nervous system (CNS) and the epidermis (cuticle) from clusters of 5-20 proneural cells that form within a monolayer of cells in the periphery of the Drosophila embryo. Most cells in the proneural clusters accumulate a high level of SuH/N^{intra}) signaling, become the epidermal precursor cells (EPCs), remain in the periphery of the embryo, and differentiate the epidermis. One or a few cells in the proneural clusters suppress SuH/N^{intra} signaling, become the neuronal precursor cells (NPCs), move inside the embryo, and differentiate the CNS (see Artavanis-Tsakonas, S. et al., 1999. Science 284, 770-776; Schweisguth, F. 2004. Curr. Biol. 14: R129-138). Production of SuH/N^{intra} signaling at any time during the differentiation of the NPCs into neurons suppresses the production of neurons Struhl, G. et al., 1993. Cell 74, 331-345; Lieber, T. et al., 1993. Genes Dev. 7, 1949-1965). However, N continues to be expressed and is required, in some other manner, during differentiation of neurons from the NPCs (Kidd, S. et al., 1989. Genes Dev., 3: 1113-1129; Fehon, R. G. et al., 1991. J. Cell Biol. 113: 657-669; Kooh, P. J. et al., 1993. Development 117: 493-507; Giniger, E. et al., 1993. Development 117, 431-440; Giniger, E. 1998. Neuron 20, 667-681; Crowner, D. et al., 2003. Curr. Biol. 13, 967-972). This raises a significant question for neurogenesis: How is production of SuH/N^{intra} signaling suppressed or prevented during differentiation of neurons from the NPCs? One mechanism that suppresses SuH/N^{intra} signaling at an early stage in the process is known. It involves Numb, an endocytic protein, thought to target N for degradation (Guo, M. et al., 1996. Neuron 17, 27-41; Spana E. P. & Doe, C. Q. 1996. Neuron, 17: 21-6; Santolini, E. et al., 2000. J Cell Biol., 151:1345-52). Here we present evidence for another mechanism covering both the early and late stages that would involve enrichment for dominant-negative N molecules lacking most of the intracellular domain or containing the SuH binding sites but not the TAD region. These molecules would titrate D1 or SuH away from the full length N, the receptor capable of producing a high rate or level of SuH/N^{intra} signaling.

### Methods

The Notch antibodies used were the following: αNT made in rabbits against the first two EGF-like repeats (Kidd, S. et al., 1989. Genes Dev., 3: 1113-1129); αN203 in rats against the first three EGF-like repeats (Wesley, C. S. & Saez, L. 2000. J. Cell Biol. 149, 683-696); αNO in rabbits against EGF-like repeats 17-21 (Kidd, S. & Lieber, T. 2002. Mech. Dev., 115: 41-51); αB in rabbits against the lin12/B repeats (a remake of the DPA antibody, Kidd, S. et al., 1989. Genes Dev., 3: 1113-1129); αVT19 in chicken and α7477 in rabbits against a bacterially made GST fusion protein containing N amino acids from 1771 to 2155 (numbers according to Kidd, S. et al., 1986. Mol. Cell. Biol., 6: 3094-3108); αNI in rabbits against the 1795 to 2157 amino acid region (Lieber, T. et al., 1993. Genes Dev. 7, 1949-1965; used only in western blots as supply is limited); the mouse monoclonal αC17.9C6 (Fehon, R. G. et al., 1990. Cell 61, 523-534) from DHSB (University of Iowa) whose epitope we have determined to lie between amino acids 1893 and 2115; α466 in guinea pigs against a bacterially made GST fusion protein containing N amino acids 2148 to 2536; αHM10 in hamsters against a bacterially made GST fusion protein containing N amino acids 2341 to 2536 (the same one described as same α2341 in Wesley, C. S. & Mok, L-P. 2003. Mol. Cell. Biol. 23, 5581-5593); and αNPCR in mouse against the 2115 to 2536 amino acid region (Lieber, T. et al., 1993. Genes Dev. 7, 1949-1965; used only to confirm patterns as its supply is nearly exhausted). Antibodies against Scabrous were generated in guinea pigs against the bacterially made GST fusion protein containing the whole Scabrous protein; SuH antibodies were made in rats (Wesley, C. S. & Mok, L-P. 2003. Mol. Cell. Biol. 23, 5581-5593); Psn antibodies were made in rabbits (a remake of the antibody described in Ye, Y. & Fortini, M. 1998. Mech. Dev., 79: 199-211); Hunchback antibodies were obtained from Drs. Nipam Patel (Patel, N. H. et al., 2001. Development 128: 3459-3472) and Paul Macdonald; and D1 (C594.9B), Elav (9F8A9), Prospero (MR1A), and 22C10, were obtained DSHB (University of Iowa). Procedures described in Sambrook, J. & Russell, D. 2001. Molecular cloning: a laboratory manual, 3rd ed., Cold Spring Harbor Press, Cold Spring Harbor, N.Y. and Harlow, E. & Lane, D. 1999. Using Antibodies. A laboratory Manual. Cold Spring Harbor Press, Cold Spring Harbor, N.Y. P. 495 were followed for making or using the antibodies.

S2-NFull, S2-N¹⁻²¹⁵⁵, and S2-N¹⁻¹⁷⁸⁹ cells have been described previously (e.g., see Bardot, B. et al., 2005. Exp. Cell Res.: 304: 202-223). Embryos were collected from cages of yw, N55e11/FM7 actGFP, D1x/TM3 actGF, P{neoFRT}82B P(Ubi-GFP)/TM3 Sb¹ P{UAS-D1-DN}TJ1 Xda-Gal4 (sorted using the GFP expression), and UAS-Ni 14EXda-Gal4 flies. Immunohistochemical staining using alkaline phosphate or horse radish peroxidase and immuno-fluorescent procedures, and in situ RNA hybridization, were performed according to Lieber, T. et al., 1993. Genes Dev. 7, 1949-1965, Corbin, V. et al., 1991. Cell 67: 311-23, and Sullivan, W. et al., 2000. Drosophila Protocols. Cold Spring Harbor Laboratory Press, Cold Spring Harbor, N.Y., p. 697. Species-specific secondary (highly cross-adsorbed) antibodies purchased from Jackson Laboratories (Bar harbor, Me.) and Molecular Probes (Invitrogen, Carlsbad, Calif.) (Alexa Fluors) were used. Green color is from Alexa Fluor 488 secondary antibody; red color from Alexa Fluor 647 secondary antibody. Western blotting and immunoprecipitation procedures described in Wesley, C. S. & Saez, L. 2000. J. Cell Biol. 149, 683-696 were followed immunohistochemical images were captured using a Nikon microscope SMZ 1500 fitted with a Spot RT Slider camera. Confocal immuno-fluorescent images were captured using the Biorad MRC 1024ES Laser scanning Imaging System. HRP and alkaline phosphates stained embryos were imaged using a SMZ 1500 stereomicroscope fitted with a Spot CCD camera from glycerol loose mounts on a plain glass slide with cover-slip props (so that the embryos can be rolled) and regular light reflected off a white base. All images were processed using Photoshop and Canvas programs. Any brightness/contrast adjustment was applied to the whole image or to the same level to all compared images.

### N Signal Patterns Described and the Procedures Used

The N antibodies used in the study and their epitope regions are shown in FIG. 1B. The N antibody signals described can be grouped into four classes: (1) signals observed with all antibodies; (2) signals observed with the extracellular domain antibodies; (3) signals observed with the Ram 23+Ankyrin repeat region antibodies; and (4) signals observed with the carboxyl terminus antibodies. αN203, αVT19, α466 signals were good representatives of (2), (3), and (4), respectively. Therefore, more data with these antibodies are shown. However, data from at least two different antibodies for each region are shown for many patterns. Signals (2) and (3) were exceedingly dynamic. Often, morphologically indistinguishable embryos showed apparently evolving patterns that were highly reproducible from batch to batch. This dynamism rendered immuno-fluorescence and confocal microscopy based procedure exceedingly inefficient and prohibitively wasteful of resources. Therefore, for basic characterization we relied on the alkaline phosphatase or horseradish peroxidase based immunohistochemical procedures, which enabled us to study thousands of identically processed, developmentally timed embryos that could be ordered according to their relative ages. We imaged embryos mounted loosely on plain glass slide using cover-slip props (so that the embryos can be rolled), illuminated by high intensity light reflected off a white base, and captured by a Spot CCD camera attached to a Nikon SMZ 1500 stereo microscope and a computer. The resolution of the images is therefore limited. However, it is sufficient to capture the patterns and dynamism, in relation to known aspects of N function. Where possible, we examined immuno-fluorescent signal patterns and found no discrepancy with signals obtained from the immunohistochemical procedures. All antibody signals described are based on at least 10 repetitions. Each repetition used large numbers of developmentally timed embryos, produced by flies entrained to the circadian cycle that yielded at least 10 embryos of a particular pattern. Thus, although not all embryos of a morphologically defined stage showed a particular pattern belonging to a dynamic series, similar or identical pattern was represented 100% of the time, at similar frequencies relative to other patterns in the series, in all repetitions. Signals observed with only one antibody have been ignored.

### Specificity and Epitope Regions of N Antibodies Used

αN203, αVT19, and α466 signals are N specific as indicated by the following experiments. We tested the epitope region specificity of antibodies in S2 cells as many exogenous N molecules expressed in embryos are rapidly cleared (Struhl, G. et al., 1993. Cell 74, 331-345; Wesley, C. S. & Mok, L-P. 2003. Mol. Cell. Biol. 23, 5581-5593). αN203, αVT19, and α466 detected only those N molecules that contained their epitope regions (N¹⁻¹⁷⁸⁹ containing 18 amino acids from the αVT19 epitope region was weakly recognized by this antibody). All other antibodies gave similar results, detecting only N molecules containing their epitope regions. The antibodies made against the different intracellular domain regions gave different signal patterns not only in vivo but also ex vivo. Therefore, we tested the specificity of these antibodies on western blots (ex vivo). All intracellular antibodies detected only those N fragments containing their epitope regions. Drosophila S2 cells expressing NFull, N¹⁻²¹⁵⁵ N¹⁻¹⁷⁸⁹, or N lacking the first 18 EGF-like repeats, NΔ1-18 EGFs were probed with different combinations of N antibodies. All three antibodies recognize NFull, α466 does not recognize N1-2155; both αVT19 and α466 did not recognize N¹⁻¹⁷⁸⁹; and αN203 did not recognize NΔ1-18 EGFs. Structures of purified N fragments were used to determine the western blotting epitope regions of intracellular domain antibodies. Western blots of N fragments 1 and 2 were probed with the different intracellular domain antibodies. The proteins were made in bacteria and purified using the Histidine or GST columns. N fragments 3-5 were probed with antibodies made against the different regions of the N intracellular domain. α7477, αC17.9C6, and αNI showed the same pattern as αVT19; αHM10 and αNPCR showed the same pattern as α466. N fragments 3-5 were made in S2 cells and purified over GST columns. The same amount of protein was loaded in all lanes for western blotting. Wild-type (WT) yw and zygotic N null N55e11/Y embryos immuno-stained (alkaline phosphatase) with different N antibodies. The results indicated that the N antibodies we have used are specific to N and detected only N molecules containing their epitope regions. The N antibody signals were also drastically reduced or eliminated in zygotic N null (N⁻/Y) embryos relative to the wild type embryos. Signals from the primary antibody minus control embryos served as the baseline for our assessment. αHM10 and α7477 also showed drastically reduced or no signals in N⁻/Y embryos.

### N Signals in the CNS

The four extracellular domain antibodies used gave strong signals in the comnuissures and connectives (neuropile) of the CNS whereas the seven intracellular domain antibodies gave weak signals, if any. Embryos were immuno-stained (horse radish peroxidase) with the different N antibodies and immuno-fluorescence and confocal microscopy images of the embryonic CNS probed with different combinations of N antibodies or an N antibody and the hunchback antibody were prepared using an ˜10× concentrated antibody preparation. The strength of the signals was assessed relative to the signals in the ventral nerve cord (VNC) and the developing cuticle in the same embryos. The relatively strong horizontal segmental signal pattern observed only with αHM10 was ignored. In the immuno-fluoresence and confocal microscopy procedure, the extracellular αN203 and αB antibodies gave strong signals in the commissures and the connectives of the CNS and weaker signals in the surrounding cells whereas the intracellular αVT19, αC17.9C6, or α466 antibodies gave uniformly low signals in all cells of the CNS. While the αC17.9C6 signals were quite similar to those of αVT19 and α466 at the most commonly used concentration range in the field ( 1/500- 1/800), it gave relatively faint signals in the commissures and the connectives of the CNS at 10× that concentration. αC17.9C6 signals were also found to be more intracellular. Single channel images of αVT19, αC17.9C6 (ascites) and α466 signals showed a weak negative image of the commissures and the connectives of the CNS. The Hunchback antibody (αHb) did not show such a negative image in the same area indicating that it is not due to any physical barriers to antibody penetration.

The above antibody signal patterns suggest that the N intracellular domain is relatively inaccessible or deficient in the commissures and the connectives of the embryonic CNS, or is present or accessible at similar levels in this tissue as well as the surrounding tissue. On the other hand, the N extracellular domain is relatively more accessible or enriched in the commissures and the connectives of the embryonic CNS.

### N Signals During NPC (Neuroblast) Specification

At the onset of lateral inhibition, αVT19 and α7477 gave very strong signals in the pre-delamination stage NPCs (neuroblasts) at the surface of the embryo when compared to the signals in the surrounding cells. The monoclonal antibody αC17.9C6 also gave stronger signals in these NPCs relative to the surrounding cells, although the overall signals were weaker than those obtained with the polyclonal αVT19 and α7477 antibodies. We attribute this difference to multiple binding of the relatively lowly expressed N molecules by the polyclonals. The dynamism of the signal pattern obtained with αVT19 and α7477 antibodies is seen in some experimental embryos. Different N antibodies or the probe for achaete (ac) RNA were used in immuno-cytochemical or in situ RNA hybridization procedures using alkaline phosphatase-conjugated secondary antibodies. The three embryos in each set were separated by not more than a few minutes and are morphologically indistinguishable. Some of the signals in some embryos could be from proneural cells as αVT19 and α7477 gave strong signals both in the NPCs and the proneural cells. The strong αVT19 and α7477 signals were very transient, disappearing even before the NPCs have completed their delamination. For identification of NPCs, we relied on (1) their relatively large size and round morphology (Campos-Ortega, J. A. & Hartenstein, V. 1997. Springer-Verlag, New York. P. 405, (2) partial correspondence with the well-known markers (see below), and (3) the low level of the expression of E(spl)C m5+m8 RNAs compared with the surrounding EPCs. The well-known horizontal, segment-wise arrays of NPCs were vaguely discernible in the αVT19 patterns, to some degree resembling the achaete (ac) RNA pattern a short time later when the achaete expression is restricted to single NPCs within the proneural cluster. This suggests that the strong αVT19 and α7477 signals might precede the restriction of achaete expression to the NPCs and delamination of the NPCs. It also suggests that although all the NPCs become part of the regular segmental arrays some time after lateral inhibition, their actual specification might not be in unison as both the achaete and αVT19/α7477 signals indicate. Our studies indicate that the strong αVT19 and α7477 signals might be the earliest markers of the NPCs.

Antibodies made against the extracellular domain also gave stronger signals in the pre-delamination stage NPCs compared with the signals in the surrounding cells. However, these signals were more transient and much weaker than the signals obtained with the αVT19 or α7477 antibodies (αN203 and αNO gave similar signals). The extracellular domain antibodies gave strong signals in localized spots near the cell surfaces and inside the delaminating/delaminated NPCs. Strong αVT19 and α7477 signals were not observed on or in these late stage NPCs. Antibodies made against the carboxyl terminus, α466 and αHM10, gave uniform signals in all cells of the embryo at these stages.

Among the many NPC markers tested, only Scabrous showed some correspondence with the αVT19 and α7477 signals in the early stage NPCs and Hunchback showed correspondence with the αN203 signals in the delaminating or delaminated NPCs. Accordingly, immunofluorescence and confocal microscopy images of doubly probed embryos showed transient overlap between Scabrous and αVT19 or α7477 signals and good overlap between Hunchback and αN203 signals. The strong αVT19 and α7477 signals in the incipient NPCs appeared to derive from these cells becoming filled with signals. On the other hand, the strong αN203 signals in the delaminating/delaminated NPCs appeared to derive from localized spots near the surface or inside these cells.

The above described signal patterns suggest that the Ram 23+Ankyrin repeat region of N is the most enriched or accessible part of N in the pre-delamination stage NPCs i.e., in the NPCs at the periphery or the surface of the embryo. The extracellular domain of N is modestly enriched or accessible in these NPCs. In the later stage NPCs, i.e., the delaminating or the delaminated NPCs, the extracellular domain of N is the most enriched or accessible part of N, in localized spots at or near the cell surface.

### N Signals at Other Stages of Embryogenesis

In the same pool of embryos used to study the CNS development, αN203, αVT19, and α466 gave very similar signal patterns at the beginning of embryogenesis, with αN203 giving the strongest signals. The N antibodies and the probe for achaete (ac) RNA were used to assess embryos at various stages of development. Some embryos were probed with the digoxigenin labeled achaete (ac) DNA and all embryos were immuno-chemically stained using alkaline phosphatase conjugated secondary antibodies. Very soon after, αN203 and αVT19 gave a similar pattern of signals that was distinct from the signal pattern of α466. αVT19 gave the strongest signals in germ cells, followed by α203, and then α466. On the other hand, αN203 gave the strongest signals in the amnio serosa and αVT19 in the sensory organ precursor cells which are the NPCs. An embryo probed for achaete RNA, a marker for proneural cells, is also shown for comparison. At a slightly earlier stage (by just a few minutes), αVT19 gave strong signals overlapping with the proneural cells; αN203 or α466 gave very weak signals. The strong αVT19 or α7477 signal domains appeared to be generally larger than the domains of the proneural cells (marked by achaete expression) suggesting that the former might define the limits within which the proneural clusters can form. All of these observations indicate that the differences in N signals obtained with antibodies specific to the extracellular domain, the Ram 23+Ankyrin repeats region, and the carboxyl terminus are not limited to the CNS development and are apparent in many types of embryonic cells and tissues keeping in line with the wide spread function for N during embryogenesis. Indeed, the differences shown in this article constitute a minor fraction of the differences observed throughout embryogenesis.

### N Signals During the Formation of Cephalic and Ventral Furrows

The cephalic furrow and the ventral furrow are formed when a band of cells in the outer layer of the embryo invaginate and move inside to form the mesodermal and endodennal primordia (Campos-Ortega, J. A. & Hartenstein, V. 1997. Springer-Verlag, New York. P. 405). There is some, if only superficial, resemblance between the processes involved in migration of cells inside by the way of cephalic or ventral furrows and NPC delamination. While N function in ventral furrow formation is known (e.g., Morel, V. & Schweisguth, F. 2000. Genes Dev. 14: 377-388), its function in cephalic furrow formation is unknown. Nevertheless, similar observations in these two similar processes provide compelling evidence for the relationship of the signals from the different N antibodies to SuH/N^{intra} signaling.

The N extracellular domain antibodies gave strong signals in the cephalic furrow as well as in other furrows forming elsewhere at the same time. Experiments were performed and signals obtained with the different N antibodies and the probes against some important components of SuH/Nintra signaling in embryos forming the cephalic furrow and completing segmentation. All embryos were immuno-chemically stained using alkaline phosphatase conjugated secondary antibodies. The developmental time from embryo 1 to 5 was just 10-12 minutes. The strong signals in these embryos were not due to the extracellular domain antibodies non-specifically accumulating in the crevices or folds as these signals preceded the furrow formation, marking the first row of cells that would later initiate formation of the furrow. Furthermore, the strong signals disappeared when the furrow was fully formed and much deeper. A similar ‘evolution’ of signals was observed at a much later stage where the crevice or fold is more extreme. The strong extracellular domain antibody signals in the crevices/folds were transient even at later stages of embryogenesis. In some experiments, extracellular domain antibody signals in the crevices/folds in stage 13 embryos disappeared in embryos that were about 30 minutes older. The intracellular domain did not give strong signals in the crevices/folds at these stages. αSuH, αPsn, and αD1 also did not give strong signals in the inter-segmental crevices/folds at these stages.

The Ram 23+Ankyrin repeat region antibodies gave strong signals in the cephalic furrow as well as in the adjacent cells. A similar signal pattern was observed with the D1 antibody. The carboxyl terminus antibodies gave almost a negative image of the extracellular domain antibody signals in the cephalic furrow and uniformly low signals elsewhere. Comparable signals were observed with the E(spl)C m5+m8 RNA probe, SuH antibody, and the Psn antibody. Cells invaginating into and forming the ventral furrow are bounded by the rows of mesectodermal cells expressing the E(spl)C genes (these are the same cells that express single-minded, Morel, V. & Schweisguth, F. 2000. Genes Dev. 14: 377-388). The developmental time from exemplary embryos compared was estimated to be just 10-12 minutes. The extracellular domain antibodies gave strong signals in cells at the center of the field of cells bounded by E(spl)C m5+m8 RNA expression, those very likely to invaginate first. Soon after, these antibodies gave strong signals within the field of cells bounded by the E(spl)C m5+m8 RNA expression and in cells within the ventral furrow. Antibodies made against the Ram 23+Ankyrin repeat region gave strong signals in a complex pattern in the initial stages of the invagination process. Near the end of the process, these antibodies gave strong signals in the single rows of cells on either side of the ventral furrow. These strong signals were coincident with the loss of E(spl)C m5+m8 expression. A closer examination of embryos at the very early stages in the process showed that not only the Ram 23+Ankyrin repeat antibodies but also the extracellular domain antibodies gave a negative image of the E(spl)C m5+m8 expression. Attempts at protein/RNA double labeling have failed so far. Even the minimal protease treatment required for RNA hybridization destroyed N proteins and the substitute acetone treatment gave very poor results, possibly due to the generally low expression of N proteins and the E(spl)C RNAs.

The carboxyl terminus antibodies gave a negative image of the extracellular domain antibody signals early in the invagination process; near the end of the process, they gave strong signals in the rows of cells on either side of the ventral furrow. SuH and Psn antibodies gave signals comparable to those of the N carboxyl terminus antibodies. On the other hand, D1 antibody signals gave signals comparable to those of the N Ram 23+Ankyrin repeat region antibodies.

The signal patterns described above suggest that the extracellular domain of N is strongly accessible or enriched in the cells invaginating into and forming the cephalic and the ventral furrows. The carboxyl terminus of N, as well as other components of SuH/N^{intra} signaling, is relatively inaccessible or deficient in these cells. There appears to be an inverse relationship between the expression of E(spl)C m5+m8 RNA and the accessibility or enrichment for the extracellular domain and the Ram 23+Ankyrin repeat regions of N.

### N Signals in the Neurogenic Embryos and Mutant Flies

If the accessibility or the level of the extracellular domain and the Ram 23+Ankyrin repeat regions of N was increased in association with the loss of SuH/N^{intra} signaling, signals from antibodies against these regions were expected to increase in neurogenic embryos which are null for SuH/N^{intra} signaling. The was examined as follows. Wild-type and neurogenic embryos at comparable stages were examined with different N antibodies. All embryos were immunochemically stained using alkaline phosphatase conjugated secondary antibodies. The neurogenic embryos were staged using the shape of the head region and the extent of the shortening of the germ band, which is quite accurate. Stages of D1 null embryos that were beginning to show the effect of loss of SuH/N^{intra} signaling showed dramatically high levels and numbers of the signals given by the N extracellular domain and the Ram 23+Ankyrin repeat. Increased signals were not observed with the carboxyl terminus antibodies. Signals by all of the N antibodies used in the study were eventually lost in zygotic N null (N⁻/Y) neurogenic embryos. However, at stages that were beginning to show the effect of loss of N, the signals given by the N extracellular domain and the Ram 23+Ankyrin repeat antibodies also dramatically increased in level and number, in a pattern comparable to the wild-type pattern. Increased signals were not observed with the carboxyl terminus antibodies. An interesting pattern could be discerned with the D1 null embryos. Signals by the Ram 23+Ankyrin repeat region antibodies initially increased in all the NPCs. Subsequently, these signals almost disappeared (similar evolution of signals was observed with the αVT19 antibody as well). Signals with the extracellular domain antibodies also increased initially, coinciding with the Ram 23+Ankyrin repeat region antibody pattern but with additional signals in localized spots. At later stages, while the signals coincident with the Ram 23+Ankyrin repeat region antibody signals disappeared, the strong signals in localized spots persisted. Similar evolution of signals was observed with αN203. We interpret the extracellular domain and the Ram 23+Ankyrin repeat region antibody signals in the D1 and N null embryos as an increase over the level of signals observed in control wild type embryos because the intensity of signals in the null embryos appeared to be greater than in the wild type embryos in the same pool even with allowance for increased numbers of NPCs.

We also examined whether or not the N extracellular domain and the Ram 23+Ankyrin repeat antibody signals increase in embryos that were manipulated to reduce SuH/N^{intra} signaling. We expressed the dominant negative D1 transgene D1-DN (Huppert, S. S. et al., 1997. Development, 124: 3283-3291) or the N RNAi construct 14E (Presente, A. et al., 2002. Genesis 34: 165-169) in a general manner using the da-Gal4 driver. Although these experiments are complicated by many factors, they clearly showed that removal of N or D1 activity results in increased signals from the extracellular domain and the Ram 23+Ankyrin repeat region antibodies but not from the carboxyl terminus antibodies. Signals were obtained with the different N antibodies in embryos manipulated to reduce the SuH/Nintra signaling and in comparable control embryos. Signals in stage 11-12 embryos expressing UAS-D1-DN driven by da-Gal4 or only da-Gal4 were assessed. Signals in stage 12-13 embryos expressing UAS-N RNAi 14E driven by da-Gal4 or only da-Gal4 were assessed. All embryos were immuno-chemically stained using the alkaline phosphatase conjugated secondary antibodies. About 20% of the UAS-D1-DN; da-Gal4 embryos showed highly deformed morphology with either no N antibody signals or strong N signals in random patterns. About 40% of the embryos (between stages 5 to 13) showed N extracellular domain and the Ram 23+Ankyrin repeat antibody signals that were stronger than those in the control embryos; the carboxyl terminus antibody signals were stronger than in control embryos until about stage 10 after which the signals were weaker than in the control embryos. The remaining embryos (stage 13 onwards) showed loss of signals with all antibodies when compared with the control embryos (the extracellular domain and the Ram 23+Ankyrin repeat regions antibodies giving more variable signals). The strong signals in the early stages might represent NFull not utilized for SuH/Nintra signaling or the time taken in this artificial system for increasing the level or accessibility of the extracellular domain and the Ram 23+Ankyrin repeat regions. The loss in signals with all antibodies at later stages was unexpected but might be a specific to the expression of the dominant negative D1 molecule that is not the same as the complete loss of D1 expression observed with the classical D1 null mutants. Only about 1% of the UAS-NRNAi; da-Gal4 embryos showed the classic neurogenic phenotype (such embryos were never observed from control crosses). About 10% of the embryos were highly deformed and were ignored. Like the classical neurogenic embryos, the RNAi neurogenic embryos showed strong signals with the extracellular domain and the Ram 23+Ankyrin repeat region antibodies and weak signals with the carboxyl terminus antibodies (relative to control embryos). In both the classical and transgenic N or D1 null/hypoactive embryos the N extracellular domain and the Ram 23+Ankyrin repeat region antibody signal increased but not the carboxyl terminus antibody signals.

If increased signals from the N extracellular domain and the Ram 23+Ankyrin repeat antibodies was sufficient for the production of neurons, the CNS was expected to be more or less developed in neurogenic embryos. This was examined as follows. Immunostaining with the neuronal marker Hunchback antibody showed signal patterns comparable to the patterns obtained with the Ram 23+Ankyrin repeat antibodies in the neurogenic embryos: the signals increased initially (possibly due to increase in the numbers of NPCs/neuroblasts) but were eventually lost. The Elav antibody, another neuronal marker, gave somewhat similar results. In these studies, signals obtained with the neuronal marker Elav antibody in the wild type (yw) and zygotic N null (N55e11/Y) neurogenic embryos were assessed. Embryos at stage 12-13 and embryos at 13 were assessed. All embryos were immuno-chemically stained using alkaline phosphatase conjugated secondary antibodies. It appeared that either the neurons failed to form fully or they failed to persist. Thus, the processes that are associated with increased signals from the N extracellular domain and/or the Ram 23+Ankyrin repeat region antibodies appear to be insufficient for either producing fully formed neurons or their stable existence. While both N null and D1 null embryos showed similar patterns, we show data for only the more rigorous N null test material. D1 null embryos are less rigorous for this hypothesis testing as D1 has N independent activity that might be required for neurogenesis (Mok, L-P., et al., 2005. BMC Dev. Biol. 5:6).

The results described above support the hypothesis that the N extracellular domain and the Ram 23+Ankyrin repeat regions become more accessible or enriched in association with loss of SuH/N^{intra} signaling. They also show that this accessibility or enrichment is not sufficient for the formation of stable neurons.

### N Signals Oil Western Blots

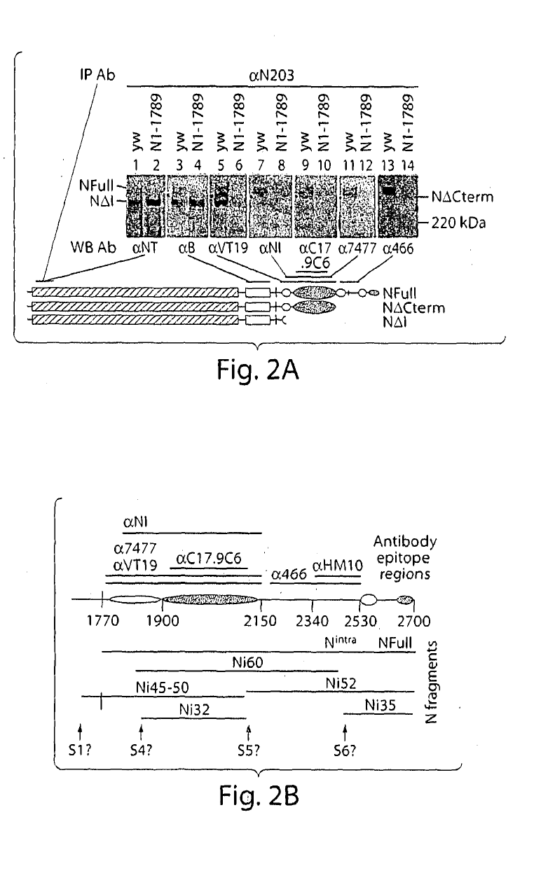

Western blotting of αN203 immunoprecipitates from embryonic extracts showed a faster migrating form of N that was recognized αNT, αB, αVT19, but not by αNI, αC17.9C6, α7477, and α466 (FIG. 2A). We will refer to this form as NΔI. Detection by αVT19 indicates that the carboxyl terminus of NΔI lies definitely after the amino acid 1771 (the end of the transmembrane domain), possibly a few amino acids after 1789 as this antibody detects NΔI better than N¹⁻¹⁷⁸⁹ (FIG. 2B, lanes 5-6). Note that, in SDS-PAGE with β-mercaptoethanol, NΔI migrates alongside N¹⁻¹⁷⁸⁹ truncated just after the end of the transmembrane domain at 1771 and faster than NΔCterm truncated just after the end of Ankyrin repeats at 2145 (see even numbered lanes FIG. 2A). As we observed quite dramatic differences in the in vivo signals obtained with αVT19 and the extracellular domain antibodies, it appears that αVT19 does not detect NΔI in vivo (if it did, the differences would be an underestimate of the actual differences). This inference is supported by the absence of obvious differences between the in vivo signals of αVT19 and α7477 that does not recognize NΔI (see FIG. 2A, lanes 11-12). In any case, detection by αVT19 distinguishes NΔI from the putative S2/S3 cleaved extracellular domain of the hetero-dimeric receptor (its unavailability being the prime reason for not detecting NΔI in our previous study, Wesley, C. S. & Saez, L. 2000. J. Cell Biol. 149, 683-696).

Similar analyses with the intracellular domain antibodies showed several N molecules that were recovered and/or detected by at least two different N antibodies and expressed at relatively significant levels (assessed in relation to the level of NFull or the house keeping protein Hsp 70). One such molecule, called Ni45-50, migrated sometimes at 45 kDa and sometimes at 50 kDa, possibly due to modification. See FIG. 2B for the structure and 2C (lanes 1 and 5) for the western blot identity (lanes 3 and 7 show the extract after the immuno-complexes were cleared, i.e., the flow through). In order to obtain nicely resolved bands, a reasonable statistical sampling of the different N fragments, and to minimize the IgG related background possible with the procedure, the immuno-precipitations were done with limiting quantities of the N antibodies. Thus, N molecules remain are expected in the flow through (FIG. 2C, lanes 3, 7). Ni45-50 was detected by aB and biotinylated in cell surface biotinylation experiments with disassociated embryonic cells. Thus, Ni45-50 appears to have the transmembrane domain. Although Ni45-50 appears to be NΔCtermTMintra (Wesley, C. S. & Saez, L. 2000. J. Cell Biol. 149, 683-696), we use a different name as it was identified by a different approach. Ni32 appears to be Ni45-50 without the amino terminus transmembrane/juxtamembrane region (see FIG. 2B for the structure and 2C lanes 1 and 5 for the western blot identity). The other molecules shown in FIG. 2B, namely Ni60, Ni52, and Ni35, were expressed at lower or variable levels than Ni45-45, Ni32, or N^{intra} (see FIG. 2C lanes 1 and 5 for their western blot identities). Note that the levels can only be assessed in relation to the level of NFull in the lanes as the different fragments transfer to the blots at increasing efficiency from the ˜400 kDa NFull to the ˜30 kDa Ni32.

The above described immuno-precipitation and western blotting analyses showed that the wild type embryos contain high levels of a N molecule composed of the epitope regions of all the N antibodies (NFull), a N molecule mostly composed of the epitope regions of the extracellular domain antibodies (NΔI), N molecules mostly composed of the epitope regions of all the intracellular domain antibodies (N^{intra}), and N molecules mostly composed of the epitope regions of the Ram 23 region+the ankyrin repeats region antibodies (Ni45-50 and Ni32). They also contain low levels of N molecules lacking the carboxyl terminus (NΔCterm), N molecules mostly composed of the epitopes regions of the carboxyl terminus antibodies (Ni52, Ni35) or N molecules composed of portions of the epitope regions of the carboxyl terminus and the Ram 23+Ankyrin repeats region antibodies (Ni60).

### Discussion

### Interpretation of In Situ and Ex Situ Signal Data

All our controls and comparisons to published reference patterns show that the antibody signals we have described derive specifically from the antigens of the antibodies used. With N antibodies, our controls show that they are specific to the epitope regions of the antibodies. Besides these controls, the extremely predictable dynamism of N signals, not only within a process but between different processes, manifest with at least three different antibodies for each region that was made in different labs or animals, also indicates signal specificity. Dynamic antibody signals derive from the enrichment or loss in the level or accessibility of the epitopes compared with a baseline level. The strong signals by the extracellular domain, the Ram23+Ankyrin repeat region, and the carboxyl terminus antibodies appear to be generally due to enrichment rather than loss in the levels or accessibility of their epitopes. The uniform and low level of the carboxyl terminus antibody signals at most stages of embryogenesis that appears to be the baseline level with all antibodies supports this inference. The rare occasions showing enrichment or loss of the carboxyl terminus antibody signals indicates that our procedures would have detected if such enrichment or loss were widespread. In the instances where the signal pattern of the intracellular domain antibodies included a weak ‘negative image’ of the signal pattern of the extracellular domain antibodies, the loss in the levels or accessibility of the intracellular epitopes is lower than the enrichment in the levels or accessibility of the extracellular epitopes as the depth of the ‘negative’ and the ‘positive’ images do not seem to match. In the processes where we can place the signal patterns in a developmental sequence, such as the differentiation of the CNS from the proneural cells, the enrichment in the levels or accessibility of the Ram 23+Ankyrin repeat region antibody epitopes preceded the enrichment in the levels or accessibility of the extracellular domain antibody epitopes. In general, however, it appears that the enrichment in the levels or accessibility of the Ram 23+Ankyrin repeat region antibody epitopes is complex and very dynamic while that of the extracellular antibody epitopes is well defined and relatively stable.

Our ex vivo immuno-precipitation and western blotting data show smaller N molecules that contain the epitope regions of some antibodies but not of others, paralleling the signal patterns observed in vivo. This correspondence suggests that the weak in vivo signals with antibodies against one N region when there were strong signals with antibodies against other N regions is due to the difference in the level rather than the accessibility of the epitopes. Thus, the enrichment for the extracellular domain signals could be due to the enrichment for NΔI. The enrichment for the Ram 23+Ankyrin repeat region signals could be due to the enrichment for Ni45-50 and/or Ni32 (with αVT19 better at detecting the former at the cell surface and αC17.9C6 the latter inside the cell). The enrichment for both the extracellular domain and the Ram 23+Ankyrin repeat region signals could be due to the enrichment for NΔCterm or the simultaneous enrichment for NΔI, Ni45-50, Ni32, and NΔCterm. The enrichment for the carboxyl terminus signals is more likely to be due to the enrichment for Ni52 and Ni35 rather than N^{intra} because we did not observe it in association with E(spl)C RNA signals or during lateral inhibition. However, N^{intra} could be the basis in some instances. In other instances, the low and uniform level of NFull represented by the carboxyl terminus antibody signals (and shared by all antibodies) appears to be permissive for the usual levels of the SuH/N^{intra} signaling. Due to our ignoring signals (1) given by single antibodies, (2) that could not be related to N functions, and (3) that could not be accurately described due to the extreme dynamism, the differences between the different antibody signals we describe are an underestimate of the actual differences in N epitope patterns during Drosophila embryogenesis. The smaller N fragments do not appear to be products of transcriptional or RNA based post-transcriptional processes (e.g., alternate splicing etc.) as the N gene lacks appropriate regulatory regions to produce them. They are likely to be produced from NFull by highly regulated proteolytic mechanisms that rapidly produces and destroys them. Otherwise, we would not have detected such dramatic differences in the signals given by antibodies against the different N regions. Our N RNAi data also supports a proteolytic mechanism. NΔCterm could be produced by the removal of Ni52 from NFull; NΔI from the removal of Ni35 and Ni60 from NFull and/or Ni32 from NΔCterm. These potential cleavage sites (S4-S6) are shown in FIG. 2B. It is possible that NFull, NΔCterm and NΔI are all substrates for S1 cleavage by Furin to make the hetro-dimeric forms. In particular, Ni45-50 could be part of a hetero-dimeric receptor as our size estimate indicates that this molecule's amino-terminus is very close to the S1 cleavage site. Thus, it is possible that while NFull functions as a collinear molecule, NΔCterm and NΔI function as hetro-dimeric molecules. The N carboxyl terminus has poly-ubuitination and PEST sites important for endocytosis and turn over (see FIG. 1A; Rechsteiner, M. 1988. Adv. Enzyme Regul., 27: 135-151; Sakata, T. et al., 2004. Curr Biol., 14: 2228-36; Wilkin M. B. et al., 2004. Curr Biol., 14:2237-44). We have shown that N molecules lacking the carboxyl terminus are deficient in both D1 independent and dependent internalization (Bardot, B. et al., 2005. Exp. Cell Res.: 304: 202-223). Thus, the enrichment for molecules lacking the carboxyl terminus (NΔCterm, NΔI, Ni45-50, Ni32) could be facilitated by the loss of endocytosis and turnover signals. On the other hand, the enrichment for molecules containing the carboxyl terminus might be suppressed by the presence of these signals, thereby explaining the uniformly low level of expression of these molecules at most stages of embryogenesis.

The N extracellular domain fragment cleaved at the S2 and S3 sites is thought to be pulled by D1 endocytosis into the NPCs, in association with increased SuH/N^{intra} signaling in the EPCs (Klueg, K. M. & Muskavitch, M. A. T. 1999. Development 112, 3289-3297; Parks, A. L. et al., 2000. Development, 127: 1373-1385; Pavlopoulos, E. et al., 2001. Dev. Cell, 1: 807-816; Struhl, G. & Adachi, A. 2000. Mol. Cell, 6: 625-636). The ex vivo NΔI molecule is not such a transendocytosed N extracellular domain fragment as it contains a small part of the intracellular domain and the transmembrane domain, i.e., it is not cleaved at the S2 or S3 sites (see FIG. 1A for the location of these sites). The in vivo N recognized by all of the extracellular antibodies and none of the intracellular antibodies is also unlikely to be such a fragment because it is produced in D1 null or N null embryos that are deficient in SuH/N^{intra} signaling, D1, or NFull. In fact, we observed increased extracellular domain signals in the neurogenic D1 null and N null embryos. However, it is possible that the in vivo N or the ex vivo NΔI is a molecule transendocytosed by a novel mechanism that is not directly dependent on D1/S2 or S3 cleavage/SuH/N^{intra} signaling but in response to these.

### Significance of the Signal Data to Tissue Differentiation in Drosophila

Our study shows that the signals from the N extracellular or the Ram 23+Ankyrin repeats region antibodies change dramatically in the course of embryogenesis, correlating with the regulation of SuH/N^{intra} signaling. These changes are possibly due to the production of N molecules composed mostly of the extracellular domain (NΔI) or the Ram 23+the Ankyrin repeats (Ni45-50 or Ni32). Such molecules are known to behave as dominant negative molecules with respect to the SuH/N^{intra} signaling by NFull: NΔI-like molecules by titrating away D1 and NΔCterm, Ni45-50- or Ni32-like molecules by titrating away SuH (Lindsley, D. L. & Zimm, G. G. 1992. The genome of Drosophila melanogaster. Academic Press, NY., p1133; Lieber, T. et al., 1993. Genes Dev. 7, 1949-1965; Lyman, D. & Young, M. W. 1993. Proc. Natl. Acad. Sci USA, 90:10395-10399; Sun, X. & Artavanis-Tsakonas, S. 1997. Development, 124: 3439-48; Jacobsen T. L. et al., 1998. Development 125:4531-40; Brennan, K. et al., 1999. Dev Biol. 216: 230-42; Wesley, C. S. & Saez, L. 2000. J. Cell Biol. 149, 683-696; Wesley, C. S. & Mok, L-P. 2003. Mol. Cell. Biol. 23, 5581-5593). Accordingly, the signals from the N extracellular or the Ram 23+Ankyrin repeats region antibodies are enriched in cells/tissues with reduced SuH/Ninta signaling. We will briefly describe below the possible significance of our data to the regulation of Drosophila tissue differentiation. Activation and suppression of SuH/N^{intra} signaling is used to specify two different cell types from a stem cell population. These cell types go on to produce two different tissues. Neurogenesis and epidermogenesis from proneural stem cells in Drosophila embryos exemplify the use of SuH/N^{inta} signaling during development.

Proneural cells that increase SuH/N^{intra} signaling become the EPCs and differentiate the epidermis. Proneural cells that suppress SuH/Nᵢₙₜᵣₐ signaling become the NPCs and differentiate the nervous system. Even a low level of SuH/N^{intra} signaling during differentiation of the NPCs, even at late stages, will suppress the production of the nervous system (Struhl, G. et al., 1993. Cell 74, 331-345; Lieber, T. et al., 1993. Genes Dev. 7, 1949-1965). This indicates that the differentiating neuronal cells retain the capacity to transduce the SuH/N^{intra} signaling but do not produce this signaling even though N and D1 are expressed in these cells and are required for completing the neuronal differentiation program (Shellenbarger, D. L. & Mohler, J. D. 1978. Dev. Biol., 62: 432-446; Kidd, S. et al., 1989. Genes Dev., 3: 1113-1129; Fehon, R. G. et al., 1991. J. Cell Biol. 113: 657-669; Kooh, P. J. et al., 1993. Development 117: 493-507; Giniger, E. et al., 1993. Development 117, 431-440; Giniger, E. 1998. Neuron 20, 667-681; Crowner, D. et al., 2003. Curr. Biol. 13, 967-972). NΔCterm, Ni45-50 and/or Ni32 molecules might initiate the suppression of SuH/N^{intra} signaling in a dominant-negative manner by titrating SuH away from NFull. However, this suppression would be only partial as NΔCterm, Ni45-50 or Ni32 molecules are capable of producing some SuH/N^{intra} signaling (Struhl, G. & Adachi, A. 1998. Cell, 93: 649-660; Wesley, C. S. & Saez, L. 2000. J. Cell Biol. 149, 683-696; Wesley, C. S. & Mok, L-P. 2003. Mol. Cell. Biol. 23, 5581-5593). In contrast NΔI is completely null for SuH/N^{intra} signaling and would also dominant-negatively suppress SuH/N^{intra} signaling by titrating D1 away from NFull. Thus, the cells or tissues requiring suppression or blockage of the SuH/N^{intra} signaling might enrich for NΔCterm/Ni45-50/Ni32 or NΔI molecules, respectively. If so, Drosophila would have adopted the simple and effective means for inhibiting biochemical reactions: producing a defective substrate that binds its ligands. This mechanism that works with the NPCs, might also work during the formation of cephalic furrow, ventral furrow, germ cells, proneural clusters, etc. Interestingly, in all these processes cells within a defined area separate and move away from their neighbors that stick together. We have shown that NFull binds D1 very strongly compared with NΔCterm or NΔI and that the SuH/N^{intra} signaling is positively correlated with binding strength (Ahimou, F. et al. 2004. J. Cell Bio.: 167: 1217-1229). Thus, the enrichment for truncated N molecules might serve both the biochemical and biophysical processes regulating tissue differentiation.

Our data show that loss of functional N genes or SuH/N^{intra} signaling might lead to production of incompletely formed or unstable neurons although there is increased levels of the extracellular domain and the Ram 23+Ankyrin repeat region epitopes as observed during normal neurogenesis. These observations indicate that the normal differentiation of the NPCs into the nervous system in the embryos might require suppression of SuH/N^{intra} signaling and the epidermis or alternate N and D1 functions that should not produce SuH/N^{intra} signaling. Indeed, our studies show that NΔCterm, Ni45-50 and/or Ni32 up regulate the expression of neurogenesis genes such as daughterless (Wesley, C. S. & Saez, L. 2000. J. Cell Biol. 149, 683-696) and D1 has neurogenesis promoting activity independent of its activity as a ligand of N (Mok, L-P., et al., 2005. BMC Dev. Biol. 5:6).

The observation that the extracellular domain and the Ram 23+Ankyrin repeat region antibody signals increase in N and D1 null embryos raises the possibility for an interesting basis for the dominance of N null mutations. Specification of two cell types during lateral inhibition is based on the relative levels of SuH/N^{intra} signaling (Heitzler, P. & Simpson, P. 1991. Cell 64: 1083-1092). Cells that produce SuH/N^{intra} signaling at a higher rate or level increase this signaling by a positive feedback mechanism to become one cell type (e.g., the EPCs). Cells that produce SuH/N^{intra} signaling at a lower rate or level, suppress this signaling to become the other cell type (e.g., the NPCs). It is possible that cells activate mechanisms that increase or suppress SuH/N^{intra} signaling depending on whether or not they have attained a certain set level of this signaling relative to their neighbors. If that level is not reached, the cells might automatically activate the mechanism that suppresses SuH/N^{intra} signaling. This kind of an auto-down regulation mechanism might explain the choice of cells for effecting lateral inhibition (in the classical sense) and the worsening symptoms with age in diseases involving N (Kalimo H. et al., 1999. Neuropathol Appl Neurobiol. 25: 257-65; Gridley, T. 2003. Hum. Mol. Genetics 12: R9-R13).

### Example 2

### Background

CADASIL (Cerebral Autosomal Dominant Arteriopathy with Subcortical Infarcts and Leukoencephalopathy) is an arterial disease that is a leading genetic cause of stroke and dementia in humans. Affected people show symptoms at middle age and die prematurely. CADASIL is caused by mutations in the Notch 3 gene encoding for a cell surface receptor. Notch receptors generate intracellular signals in response to ligand binding that are required for tissue differentiation in all animals. Knockout mice data indicate that the Notch 3 gene is required for the production and maintenance of cerebral arteries. Mutations in CADASIL patients have been found in almost all functional regions of the Notch receptor. These data suggest that loss of Notch 3 function is the cause of the CADASIL disease. If this were the case, all CADASIL mutations are expected to be deficient in ligand binding or signaling. This expectation has not been met in in vitro studies done so far using conventional methods. It is important to determine whether or not this unexpected result is due to the limitations of the methods employed in order to avoid erroneous rejection of the most likely cause suggested by a preponderance of evidence. It is also important to develop a model based on the mechanism of Notch 3 function or metabolism for a better understanding of the development of the CADASIL disease.

Recently, an extremely sensitive assay combining Atomic Force Microscopy and pharmacologic treatment was developed to measure the binding strength and the rate of Notch signaling in live cells. This method showed that Drosophila Notch receptors with CADASIL-like mutations, that were not expected to affect ligand binding or signaling based on results from conventional methods, actually affects them very significantly. This new procedure is applied to a sample of human Notch 3 receptors carrying CADASIL associated mutations expressed in human cultured cells to test whether all CADASIL mutations are deficient in ligand binding and the rate of Notch 3 signaling.

Mammalian cells expressing the human Notch 3 receptor produce truncated molecules that resemble Notch molecules involved in the Drosophila auto-down-regulation mechanism activated in response to reduced levels of Notch signaling. This resemblance suggests that the CADASIL disease develops or worsens due to increased activity of the Notch 3 auto-down-regulation mechanism. Conventional cyto-chemical and molecular experiments are conducted with human cultured cells expressing the human Notch 3 receptor and its ligand to find out more precisely the structure of the truncated Notch 3 molecules produced and the effect of CADASIL mutations on the levels of these molecules.

CADASIL is a genetically dominant disease causing stroke and dementia in a significant number of people. Molecular features of the disease can be detected in childhood but the clinical symptoms manifest in middle age and result in premature death. The symptoms are more severe in homozygous patients. Brain cells primarily affected are the vascular smooth muscle cells of the arteries that progressively degenerate. The disease is caused by mutations in the Notch 3 gene. Notch genes encode for evolutionarily conserved cell surface receptors that generate tissue differentiation and maintenance signals in response to ligands binding their extracellular domain. Mutations in CADASIL patients have been discovered in almost all functionally important regions of Notch 3. Notch 3 Knock out mice show defects that indicate a significant role for Notch 3 in arterial differentiation and vascular smooth muscle cell maturation. Transgenic mice expressing a Notch 3 receptor with a CADASIL mutation develop vascular features of the CADASIL disease. We investigated whether the CADASIL disease is caused by the loss of Notch 3 signaling and that all mutated receptors are not only functionally deficient but also dominant negative. In vitro studies that explored the signaling part of the hypothesis have reported that not all mutated Notch 3 receptors are deficient in ligand binding or signaling capability raising the possibility that either the hypothesis is wrong or the conventional methods used in the studies were not sensitive. This issue has to be resolved in order to properly pursue the cause of the CADASIL disease. Another approach to understand the development of the CADASIL disease is to explore mechanisms that explain the distinctive features such as the accumulation of Notch 3 molecules without the intracellular domain and the relatively slow progression of the disease. The two approaches could help us better understand CADASIL disease, strokes, and cognitive impairment.

The Notch receptor in Drosophila functions similarly to the Notch receptors in mammals. Indeed, much of our knowledge of mammalian Notch receptors is derived from the Drosophila Notch receptor. We have developed a very sensitive procedure that uses atomic force microscopy and pharmacologic intervention with live cells for determining the ligand binding strength of Notch receptors and the rate of Notch signaling. This procedure applied to the Drosophila Notch receptor has shown that mutations comparable to the CADASIL mutations that were found by conventional methods to be not deficient in ligand binding or signaling are indeed very deficient in both aspects. A stunning discovery was that Notch signaling at a contact point peaks within minutes of ligand binding and falls to zero in just 10 minutes! Mutated receptors bind ligands weakly and signal slowly. Flies expressing loss of function alleles, including alleles producing CADASIL-like mutant receptors, accumulate Notch molecules lacking a portion of the intracellular domain (NΔCterm) and most of the intracellular domain (NΔI). NΔCterm and NΔI produce little or no Notch signaling, are very stable due to poor internalization, accumulate only in differentiating cells that suppress Notch signaling, and can suppress Notch signaling by promoting degradation of the full length Notch receptor or titrating away ligands. Human Notch 3 receptors expressed in mammalian cultured cells produce molecules that appear to be the byproducts of the production of NΔCterm- and NΔI-like molecules. These data suggest the following, with respect to the development of the CADASIL disease: Mutations in Notch 3 reduce the ligand binding strength or interfere with intracellular signal transduction. The consequent reduction in Notch 3 signaling leads to the production of Notch 3 molecules lacking a portion of the intracellular domain (hN3ΔCterm) that in turn leads to the production of Notch 3 molecules lacking most of the intracellular domain (hN3ΔI). hN3ΔI accumulates due to poor internalization and turnover, gradually worsening the dominant negative effect of ligand titration. Disease symptoms manifest after a threshold of tolerance for the loss of Notch 3 signaling is crossed.

CADASIL mutations in three different extracellular regions of the human Notch 3 receptor are examined to determine whether the mutations reduce ligand binding strength and signaling in human cultured cells using the sensitive atomic force microscopy and pharmacologic intervention based method. Vascular smooth muscle cells that are the primary target of the CADASIL disease will also be used in these experiments. Whether NΔCterm-like and NΔI-like molecules are produced from the human Notch 3 receptor expressed in human cultured cells is also examined along with whether the levels of these molecules are affected by CADASIL mutations. Conventional cyto-chemical and molecular procedures are used for this analysis.

### The Basic Features of the CADASIL Disease

CADASIL (Cerebral Autosomal Dominant Arteriopathy with Subcortical Infarcts and Leukoencephalopathy) is an arterial disease that is a leading genetic cause of stroke and dementia in people. Clinical symptoms start to manifest around 40 years of age and include migraine, mood disorders with depression, recurrent strokes, progressive cognitive and intellectual impairment, dementia, and premature death Kalimo, H, et al., 1999. Neuropathol Appl Neurobiol. 25: 257-65). Brain cells primarily affected are the vascular smooth muscle cells of small and middle-sized arteries that degenerate. The consequent impaired blood supply leads to brain tissue necrosis. Conglomerates of tiny granules, called granular osmiophilic material or GOM, accumulate within the basement membrane of the affected cells or in the surrounding matrix (Tournier-Lasserve, E. et al., 1993. Nat Genet. 3:256-9; Chabriat, H. et al., 1995. Lancet. 346:934-9; Ruchoux, M. M. et al., 1995. Acta Neuropathol. 89: 500-512; Kalimo, H, et al., 1999. Neuropathol Appl Neurobiol. 25: 257-65; Abe, K. et al., 2002. Ann. N.Y. Acad. Sci., 977: 266-272). Vascular smooth muscle cells of exa-cerebral arteries are also affected; in fact, molecular features apparent in skin biopsies are used in early diagnosis (Ruchoux, M. M. et al., 1995. ActaNeuropathol. 89: 500-512; Ebke, M, et al., 1997. Acta Neurol Scand. 95: 351-7; Mayer, M. et al., 1999. J Neurol. 246:526-32; Ruchoux, M. M. et al., 2000. Ann N Y Acad Sci. 903:285-92; Joutel, A. et al., 2001 Lancet. 358: 2049-51). CADASIL is a slowly progressing disease with the late onset of symptoms caused by accretion of effects rather than latency (Kalimo, H, et al., 1999. Neuropathol Appl Neurobiol. 25: 257-65).

### The Cause of the CADASIL Disease

CADASIL patients carry mutations in the Notch 3 gene. Mice expressing Notch 3 receptors with mutations found in CADASIL patients show characteristic features of the CADASIL disease (Joutel, A. et al., 1996. Nature 383:707-710; Joutel, A. et al., 2002. In Notch from Neurodevelopment to Neurodegeneration. Springer-Verlag, Berlin, pp 143-156; Ruchoux, M. M. et al., 2003. Am J Pathol. 162:329-42). One such feature is the accumulation of the extracellular portion of the Notch 3 protein product in the affected tissues (Ruchoux, M. M. et al., 2003. Am J Pathol. 162:329-42; Joutel, A. et al., 2000. J Clin Invest. 105:597-605). Notch 3 knockout mice show defects in the structure, development, and function of arteries and vascular smooth muscle cells (Gridley, T. 2003. Hum. Mol. Genetics 12: R9-R13; Domenga, V. et al., 2004. Genes Dev. 18:2730-5). Expression and other studies also support arole forNotch 3 in arterial differentiation of vascular smooth muscle cells (Joutel, A. et al., 2000. J Clin Invest. 105:597-605; Leimeister, C. et al., 2000. Mech Dev. 98:175-8; Villa, N. et al., 2001. Mech Dev. 108:161-4; Prakash, N. et al., 2002. Exp Cell Res. 278: 31-44; Wang W, Prince C Z, Mou Y, Pollman M J. 2002. J Biol Chem. 277:21723-9; Shawber, CJ & Kitajewski, J. 2004. Bioessays. 26: 225-34). These data indicate that a disruption in Notch 3 function is the cause of the CADASIL disease. A CADASIL patient homozygous for a Notch 3 mutation manifests more severe phenotypes than a patient heterozygous for the same allele (Tuominen, S. et al., 2001. Stroke. 32: 1767-74). This observation indicates that the CADASIL disease is caused by the mutant Notch 3 alleles acting as classic dominant alleles with dosage dependent effects, in addition to any dominant negative effect on the wild type allelic partner.

### The Basic Features of Notch Receptors

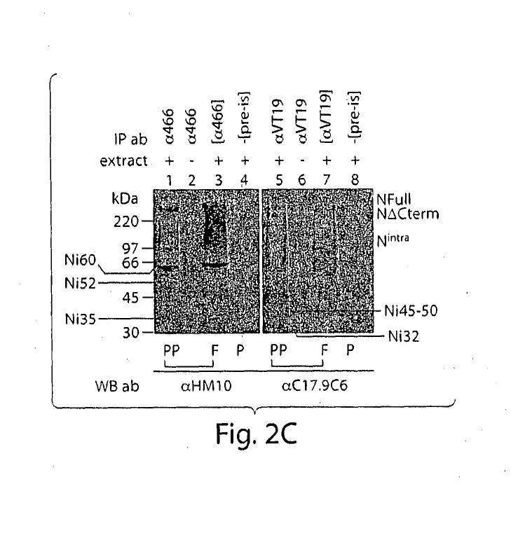

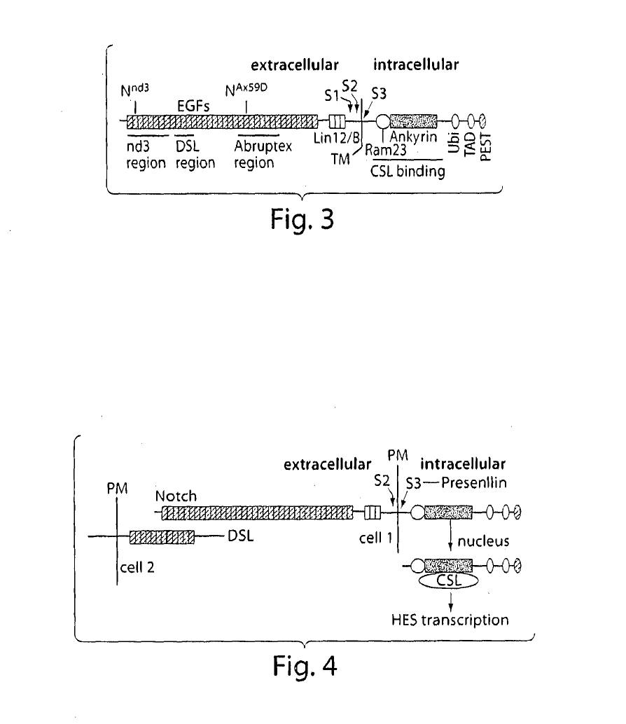

Notch genes encode for cell surface receptors that generate intracellular signals in response to binding of ligands. These signals enable the differentiation and maintenance of various tissues and organs, including heart, arteries, and the nervous system (Mumm, J. S. & Kopan, R. 2000. Dev. Biol. 228: 151-165; D'Amore, P. A. & Ng, Y. S. 2002. Cell. 110: 289-92). Notch receptor functions are highly conserved in evolution, functioning similarly in all animals from humans to Drosophila flies to Caenorhabditis elegans worms (Mumm, J. S. & Kopan, R. 2000. Dev. Biol. 228: 151-165; Greenwald, I. 1998. Genes Dev. 12:1751-62; Artavanis-Tsakonas, S. et al., 1999. Science 284: 770-776; Schweisguth, F. 2004. Curr Biol. 14: R129-38). Mammals have four Notch genes, Notch 1 to Notch 4, which function similarly, possibly in different contexts (Mizutani, T. et al., 2001. Proc Natl Acad Sci USA. 98: 9026-31; Saxena, M. T. et al., 2001. Biol Chem. 276: 40268-73; Kopan, R. 2002. J. Cell Sci. 115: 1095-1097). Drosophila has one Notch receptor. The general structure of Notch receptors and features relevant to these experiments are shown in FIG. 3.

The primary Notch ligands are Delta and Jagged in mammals, Delta and Serrate (homolog of Jagged) in Drosophila, and Lag-2 in C. elegans (DSL ligands). These ligands bind Notch in the DSL region (FIG. 3). Jagged 1, Jagged 2, Delta 1, Delta 2, Delta 3, and Delta 4 are considered to be the DSL ligands of the four mammalian Notch receptors (Gridley, T. 2003. Hum. Mol. Genetics 12: R9-R13; Hicks, C. et al., 2000. Nat Cell Biol. 2: 515-20) Delta and Serrate often function in a mutually exclusive manner in Drosophila (Doherty, D. et al., 1996. Genes Dev. 10: 421-34). A similar manner of function might also be true with mammalian DSL ligands (Lindsell, C. E. et al., 1996. Mol Cell Neurosci. 8: 14-27; Shimizu, K. et al., 2000. Biochem Biophys Res Commun. 276:385-9). Delta 1 and Jagged 1 have been used to study Notch 3 signaling (Haritunians, T. et al., 2002. Circ. Res. 90: 506-508; Karlstrom, H. P. et al., 2002. Proc. Natl. Acad. Sci. USA, 99:17119-17124; Joutel, A. et al., 2004. Am J Hum Genet. 74:338-47. Epub 2004 Jan. 8; Peters, N. et al., 2004. Exp Cell Res. 299: 454-64). However, Jagged 1 is thought to be the more likely ligand of Notch 3 (Villa, N. et al., 2001. Mech Dev. 108:161-4; Joutel, A. et al., 2004. Am J Hum Genet. 74:338-47. Epub 2004 Jan. 8). The Notch intracellular domain binds many proteins that transduce or regulate Notch signaling. Chief among them are the CSL DNA binding proteins (CBF1/RBPjk in mammals, Suppressor of Hairless in Drosophila, and Lag-1 in C. elegans; see FIG. 3 for CSL protein binding region in Notch).

### The Mechanism of Notch Signaling

When a DSL ligand binds Notch, Kuzbanian/TACE ADAM metalloprotease cleaves the extracellular domain at the S2 site (see FIG. 3). This cleavage is followed by Presenilin/g-secretase mediated cleavage at the S3 site to release the Notch intracellular domain (N^{intra}/NICD) from the membrane. N^{intra}/NICD translocates to the nucleus, and in association with CSL DNA binding protein activates expression of the Enhancer of split Complex/HES target genes (Humm, J. S. & Kopan, R. 2000. Dev. Biol. 228: 151-165; Artavanis-Tsakonas, S. et al., 1999. Science 284: 770-776; Schweisguth, F. 2004. Curr Biol. 14: R129-38; Kopan, R. 2002. J. Cell Sci. 115: 1095-1097). This mechanism is shown in FIG. 4. Mutations in the ligand-binding region result in the loss of ligand binding ability and Notch signaling (Joutel, A. et al., 2004. Am J Hum Genet. 74:338-47. Epub 2004 Jan. 8; Peters, N. et al., 2004. Exp Cell Res. 299: 454-64; de Celis, J. F. et al., 1993. Proc. Natl. Acad. Sci. USA. 90:4037-41; Brennan, K. et al., 1997. Genetics 147: 177-188; Li, Y. & Baker, N. E. 2001. Curr Biol. 11: 330-8)). Two additional regions in the extracellular domain affect DSL ligand binding or Notch signaling. The Abruptex region (see FIG. 3) is the site of modification by the Glycosyl transferase Fringe proteins and this modification promotes Notch signaling by Delta while suppressing Notch signaling by Serrate or Jagged (Hicks, C. et al., 2000. Nat Cell Biol. 2: 515-20, Moloney, D. J. et al., 2000. Nature 406: 369-375; Bruckner, K. et al., 2000. Nature 406: 411-415; Ju, B. G. et al., 2000. Nature 405: 191-195; de Celis, J. F. & Bray, S. J. 2000. Development. 127:1291-302; Haines, N. & Irvine, K. D. 2003. Nat Rev Mol Cell Biol. 4:786-97). Although mutations in this region result in phenotypes that appear to be due to gain in Notch signaling, complementation analyses against Notch gene deletions clearly indicate aloss in Notch signaling (Brennan, K. et al., 1997. Genetics 147: 177-188). Thus, these phenotypes are complex outcomes that are somehow based on the loss of Notch function. Mutations in the amino terminal nd3 region (see FIG. 3) produce classic loss of Notch signaling phenotypes (Shellenbarger, D. L. & Mohler, J. D. 1975. Genetics 81: 143-162; Lyman, D. & Young, M. W. 1993. Proc. Natl. Acad. Sci. USA 90: 10395-10399). Our studies show that it is likely to be due to the loss of Notch receptor clustering that is important for high rates of Notch signaling or its down-regulation (Bardot, B. et al., Exp. Cell Res. 304: 202-223).

Notch signaling is generally used to make two kinds of tissues from a population of stem cells: Cells with a high rate increase it further by activating a positive feedback mechanism and turn on genes for differentiation of one kind of tissue; cells with a lower rate reduce it further by activating a negative feedback mechanism and turn on genes for making the alternate tissue. Both the elimination of Notch signaling and the turning on of a different set of genes are important for making the alternate tissue.

The Basic Features of Notch 3 Mutations Associated with the CADASIL Disease

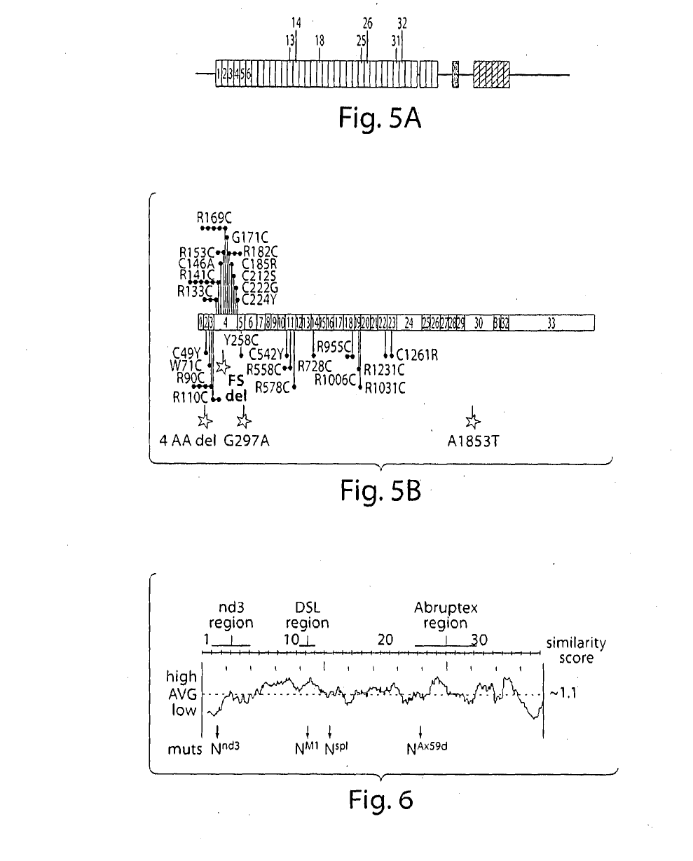

The most striking molecular feature in all CADASIL patients is the accumulation of the Notch 3 extracellular domain (Ruchoux, M. M. et al., 2003. Am J Pathol. 162:329-42; Joutel, A. et al., 2000. J Clin Invest. 105:597-605). Transendocytosis of the Notch extracellular domain into Delta expressing cells or “Delta pulling” is known to promote Notch signaling (Klueg, K. M. & Muskavitch, M. A. T. 1999. Development 112, 3289-3297; Parks, A. L. et al., 2000. Development 127: 1373-1385; Struhl, G. & Adachi, A. 2000. Mol. Cell 6: 625-636; Pavlopoulos, E. et al., 2001. Dev. Cell 1: 807-816). Thus, the extracellular domain and the intracellular domain could get separated and have independent metabolism that is affected in CADASIL patients. But this is expected with excess Notch signaling and will not explain extracellular domain accumulation with mutations in the DSL binding region. A crucial test for the validity of any model for the development of the CADASIL disease is a mechanism for the accumulation of the Notch 3 extracellular domain without the concomitant accumulation of the Notch 3 intracellular domain. The majority of mutations associated with the CADASIL disease are single missense, small in-frame deletions, or splice site alteration in the extracellular EGE-like repeats (Joutel, A. et al., 1997. Lancet 350:1511-1515; Dichgans, M. et al., 2000. Eur J Hum Genet. 8:280-5; Oberstein, S. A. et al., 1999. Neurology. 52: 1913-5; Oliveri, R. L. et al., 2001. Arch Neurol. 58: 1418-22; Dichgans, M. et al., 2001. Neurology 57: 1714-7; Joutel, A. et al., 2000. Neurology. 54: 1874-5; Dotti, M. T. et al., 2004. Arch Neurol. 61:942-5). There is a strong clustering of the mutations in the amino terminus (FIG. 5). This clustering might be due to this region (nd3 region) being less important for function compared with the DSL, Abruptex, or intracellular regions as its is less conserved over evolutionary time (FIG. 6). Patients might be preferentially sampled simply because they survive longer or develop identifiable symptoms. Alternatively, the amino terminus might be a mutational hot spot. In one study 33 out of 45 CADASIL mutations and in another 43 out of 43 showed a C to T transition affecting the CpG dinucleotides (Joutel, A. et al., 1997. Lancet 350:1511-1515; Dichgans, M. et al., 2000. Eur J Hum Genet. 8:280-5).

The majority of CADASIL mutations replaces or adds a cysteine residue, resulting in an odd number of cysteines in an EGF-like repeat (Joutel, A. et al., 1997. Lancet 350:1511-1515). This has raised the possibility that the primary cause of the CADASIL disease is a Notch 3 EGF-like repeat with an odd number of cysteines that interfere with Notch receptor trafficking or turnover (Donahue, C. P. & Kosik, K. S. 2003. Genomics. 83: 59-65). But, this cannot be true as mutations not involving a cysteine mutation, or even an EGF-like repeat, are described from CADASIL patients (mutations with stars in FIG. 5; (Joutel, A. et al., 1996. Nature 383:707-710; Mazzei, R et al., 2004. Neurology. 63: 561-4)). There are six invariant cysteines per ˜40-amino acid long EGF-like repeat in an array of 34 EGF-like repeats. It is possible that cysteine changes are more likely to affect Notch 3 function and the chances are pretty slim for getting a change to an even number (4 or 8 for example) through a small deletion of less than 10 amino acids described so far (greater than 15 would be required on average) or a double mutation within a tiny genomic segment encoding the Notch 3 EGF-like repeat array. Thus, just like the clustering of mutations in the amino terminus, mutations to an odd number of cysteines might be the red herring of the CADASIL disease.

Five observations indicate that the Notch 3 mutations in CADASIL patients are loss of function mutations. One, a frame shift deletion that truncates Notch 3 to about 5% its length is reported from a CADASIL patient (Dotti, M. T. et al., 2004. Arch Neurol. 61:942-5). Most proteins truncated to 5% their size are nulls. Two, a mutation in the Ankyrin repeat region is reported from a CADASIL patient (Joutel, A. et al., 1996. Nature 383:707-710). This region is the most conserved Notch region and is absolutely required for Notch signaling (Lieber, T. et al., 1993. Genes Dev. 7: 1949-1965). Three, CADASIL mutations is reported from almost all regions of Notch (see FIG. 5). Loss of function is the more logical expectation than gain to the same phenotype by different kinds of mutations in different functional regions. Four, Notch 3 knock out mice show defects in differentiation and maturation of the cerebral vascular smooth muscle cells (Gridley, T. 2003. Hum. Mol. Genetics 12: R9-R13; Domenga, V. et al., 2004. Genes Dev. 18:2730-5). Five, the onlyregion where true gain of Notch signaling mutations have been reported is the Lin12/B repeats (Brennan, K. et al., 1997. Genetics 147: 177-188). A CADASIL mutation in the Lin12/B repeats of Notch 3 has not been reported. This might be a significant clue because the sample size of CADASIL mutations is not all that small and mutations in the Lin12/B repeats are frequently isolated from Drosophila and C. elegans.

### Not all CADASIL Mutations Show Loss of Notch 3 Signaling in Conventional In Vitro Studies

If loss of Notch 3 signaling is the cause of the CADASIL disease, all Notch 3 receptors with CADASIL mutations should show reduced signaling capability. Four studies have examined this hypothesis using conventional in vitro methods. One study has reported that the CADASIL mutations in the ligand binding DSL region as well as those in the amino terminal nd3 region have no effect on ligand binding or signaling (Haritunians, T. et al., 2002. Circ. Res. 90: 506-508). Another study has reported that a CADASIL mutation in the nd3 region impairs trafficking to the cell surface but does not affect ligand binding and signaling once at the cell surface (Karlstrom, H. P. et al., 2002. Proc. Natl. Acad. Sci. USA, 99:17119-17124). Two studies have reported adverse effects on ligand binding and signaling with mutations in the DSL region (or mutations affecting trafficking) but not with mutations in the nd3 or the Abruptex regions (Joutel, A. et al., 2004. Am J Hum Genet. 74:338-47. Epub 2004 Jan. 8; Peters, N. et al., 2004. Exp Cell Res. 299: 454-64). As it is obvious, loss of Notch signaling is not a common feature of all mutated Notch 3 receptors examined, either in all the studies combined or within a single study. Thus, one could conclude that reduced Notch 3 signaling is not the cause of the CADASIL disease. However, such a conclusion would be premature and runs the risk of becoming a costly Type II error, acceptance of a false null hypothesis (loss of Notch 3 signaling is the not the cause of the CADASIL disease).

Possible Sources for the Type II Error in the above In vitro Studies Based on Conventional Methods

i). The form of the DSL ligands used: Notch receptors and DSL ligands are membrane-anchored proteins. Secreted Delta is a very poor activator of Notch signaling when compared with the membrane anchored Delta in both Drosophila and mammalian systems (Fehon, R. G. et al., 1990. Cell 61, 523-534; Mishra-Gorur, K. et al., 2002. J Cell Biol. 159:313-24; Shimizu, K. et al., 2002. EMBO J. 21: 294-302). The in vitro studies mentioned in the previous section used secreted Delta or Jagged extracellular domain fused to an antibody Fc region that was clustered using an antibody against the Fc region to simulate the required multimerization. These studies did not consider membrane anchorage or other aspects dependent on the Delta or Jagged intracellular domains.

ii). Yes/No binding assay: The above studies determined only whether a ligand binds or not. The methods used were not capable of measuring quantitative differences in the ligand binding strength between the wild type and the mutant Notch 3 receptors. A method for doing this was not available until now.

iii). The binding strength and signaling: Due to technical difficulties, the above studies examined ligand binding and signaling in separate assays. Thus, they did not take into consideration the effect of ligand binding strength on Notch 3 signaling. Our studies show that this effect is considerable.

iv). The incubation time: The above studies typically used two days of incubation with the ligand for studying Notch 3 signaling. This is might be too long. In Drosophila, Notch signaling in Notch/Delta cell aggregates decreases rapidly after one hour (Mishra-Gorur, K. et al., 2002. J Cell Biol. 159:313-24). At individual Notch cell/Delta cell contact points, it reaches a maximum within minutes after ligand binding and apparently shuts down in 10 minutes.

v). The indicator of Notch 3 signaling: The above studies used target gene expression from reporter constructs to determine the signaling capabilities of the mutated Notch 3 receptors. This was the best available assay but it is subject to saturation and feed back regulation that would mask even significant differences, particularly over long incubation periods. The rate of Presenilin/γ-secretase cleavage of the Notch receptor is a more accurate indicator of the signaling capability that can reveal even subtle differences.

Our data showed a very sensitive method that can overcome the above limitations and measure the ligand binding strength and signaling capabilities of the wild type and mutant Notch 3 receptors. The data also suggested a mechanism for the development of the CADASIL disease that incorporates its three essential features: similar effects with all Notch 3 mutations, progressive worsening of symptoms, and the accumulation of the Notch 3 extracellular domain. These data are presented first followed by the sensitive method.

### The Structures of Notch Molecules in Drosophila

Three ligand-binding forms of the Notch receptor are produced during Drosophila development: (1) The full length Notch molecule (NFull) that contains the transcription activation domain (IAD) and generates high levels of Notch signaling; (2) NΔCterm that lacks the region carboxyl terminus of the Ankyrin repeats, therefore also the TAD, but contains the SuH binding sites and generates a low level of Notch signaling; and (3) NΔI that lacks both the TAD and the SuH binding sites and cannot generate any Notch signaling. The Notch receptors that generate Notch signaling in mammals are the hetero-dimeric forms of the full length extracellular and the intracellular domains generated by the S1 cleavage in the golgi (see FIG. 3; (Logeat, F. et al., 1998. Proc Natl Acad Sci USA. 95: 8108-12)). NFull that generates high levels of Notch signaling in Drosophila is collinear (Kidd, S. & Lieber, T. 2002. Mech Dev. 115:41-51). The reason forthis difference is notunderstood but may be related to the role of adhesion in Notch signaling in Drosophila. Our studies suggested that NΔCterm might function in the hetero-dimeric form.

Results indicated that NΔCterm and NΔI were produced in the wild-type embryos (yw) in addition to NFull. Antibodies used for these experiments were: αNT, αB αVT19, αC17.9C6, α7477, and α466. Note that NFull is detected by all antibodies; NAΔterm is detected by all antibodies except the carboxyl terminus α466 antibody; and NΔI is detected by all the extracellular domain antibodies as well as the intracellular αVT19 antibody (which detects intracellular epitopes close to the transmembrane domain) but not other intracellular antibodies. The extensive immuno-precipitation (IP) and western blotting (WB) analyses with multiple antibodies revealed Notch molecules composed mostly of the intracellular regions that appear to be related to the production or the activities of NFull, NΔCterm, or NΔI.

### Production of NΔCterm and NΔI in Embryos and Cultured Cells.

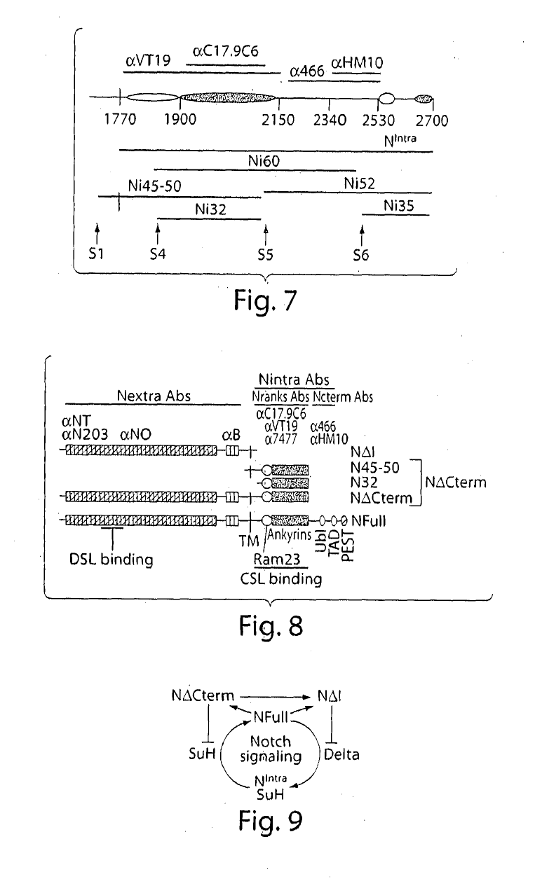

NΔCterm appears to be produced from NFull by cleavage at the S5 site (see FIG. 7 for the cleavage sites). Ni45-50 appears to be produced by the cleavage of NΔCterm at the S1 site, or NFull at the S5 and S1 sites. Ni32 appears to be produced by the cleavage of Ni45-50, the hetero-dimeric NΔCterm, or the collinear NΔCterm at the S4 site. NΔI appears to be produced by the cleavage of the collinear NΔCterm at the S4 site as a byproduct of Ni32 production, or by the cleavage of NFull at the S6 and S4 sites (in that order). These conclusions are supported by the data including the following. One, Delta and NFull interaction produces not only N^{intra} but also Ni45-50 as an auto-down-regulatory response. Two, Delta and NΔCterm interaction produces Ni32 as the activated signaling molecule. Three, cell surface biotinylation experiments with embryonic (yw) or cultured cells show that NΔCterm-like receptors (N¹⁻²¹⁵⁵) and Ni45-50 are at the cell surface but not Ni32 indicating that Ni45-50 and the S1 Notch extracellular domain might also form a hetero-dimeric NΔCterm receptor. Four, NΔCterm and Delta cells remain associated for more than seven hours (even over night) despite Ni32 production whereas NFull and Delta cells dissociate in two hours concomitant with production (Bardot, B. et al., Exp. Cell Res. 304: 202-223). Five, clusters of NΔCterm at contact points with Delta cells are often detected only by the extracellular (Nextra) domain antibodies and not by the intracellular (Nintra) domain antibodies (all NFull clusters are detected by both antibodies until their disappearance). Only the extracellular domain antibodies recognized some N¹⁻²¹⁵⁵ clusters induced by DI. Six, high levels of an NΔI-like molecule was observed in flies carrying the NΔCterm-like N^{60g11} allele. N^{60g11} flies accumulate extracellular molecules. Points 4, 5, and 6 together indicate that NΔCterm is cleaved at an intracellular site leaving the extracellular domain anchored to the membrane (NΔI), and even bound to Delta Seven, the presence of Ni60 and Ni35 in embryos (see FIG. 7) indicate that NΔI could also be produced from NFull.

FIG. 8 shows the forms of Notch relevant to these experiments and the antibodies used to detect them, grouped based on the patterns of strong signals. The carboxyl terminus antibodies (Ncterm Abs) generally give a low level of uniform signals that is shared by all antibodies and show the distribution of NFull. Strong signals by the extracellular domain antibodies (Nextra Abs) show enrichment for NΔI over the basal level of NFull. Strong signals by the Ram 23 and the Ankyrin repeats region antibodies (Nranks Abs) show enrichment for Ni45-50 or Ni32 over the basal level of NFull. Signals by both the Nextra Abs and Nranks Abs groups show the enrichment for NΔCterm. Since the enrichment and activities of NΔCterm, N45-50 or N32 are tightly linked or sequential, we will refer to them collectively as NΔCterm. All Notch antibodies used in our studies are specific to the epitope regions as determined by western blotting and immuno-staining in vivo and in vitro experiments with wild type and mutants deleted for the specific regions.

### The Activities of NFull, NΔCterm and NΔI in Embryos and Cultured Cells.

NFull and N^{intra} have the CSL binding sites and the transcription activation (TAD) domain (see FIG. 8). They are strong generators of Notch signaling that suppresses neurogenesis. NΔCterm and related molecules (Ni45-50 and Ni32) have activities that promote neurogenesis. This activity was confirmed by our microarray analysis. However, the activity of NΔCterm, Ni45-50, and Ni32 is dominant negative suppression of NFull activity and Notch signaling. The Drosophila CSL, Suppressor of Hairless, is not just a transducer but also a target of Notch signaling and a stabilizer of NFull. NΔCterm stability is unaffected by Suppressor of Hairless levels. When Suppressor of Hairless is titrated away from NFull by NΔCterm, Ni45-50, or Ni32, NFull is ubiquitinated in the carboxyl terminus region (Ubi in FIG. 8) and degraded. This leads to loss of Suppressor of Hairless that leads to further loss of NFull and thereby to loss of Notch signaling (Bardot, B. et al., Exp. Cell Res. 304: 202-223). NΔI-like molecules dominant negatively suppress Notch signaling by titrang Delta away from NFull (Lieber, T. et al., 1993. Genes Dev. 7: 1949-1965; Sun, X. & Artavanis-Tsakonas, S. 1997. Development. 124: 3439-48; Jacobsen, T. L. et al., 1998. Development 125:4531-40; Brennan, K. et al., 1999. Dev Biol. 216: 230-42). In fact, over-expression of NΔI-like molecules is routinely used in the field to reduce Notch signaling. FIG. 9 shows the two dominant negative mechanisms auto-regulating NFull activity and Notch signaling.

### Distribution of NFull, NΔCterm, NΔI During Neurogenesis in Drosophila Embryos

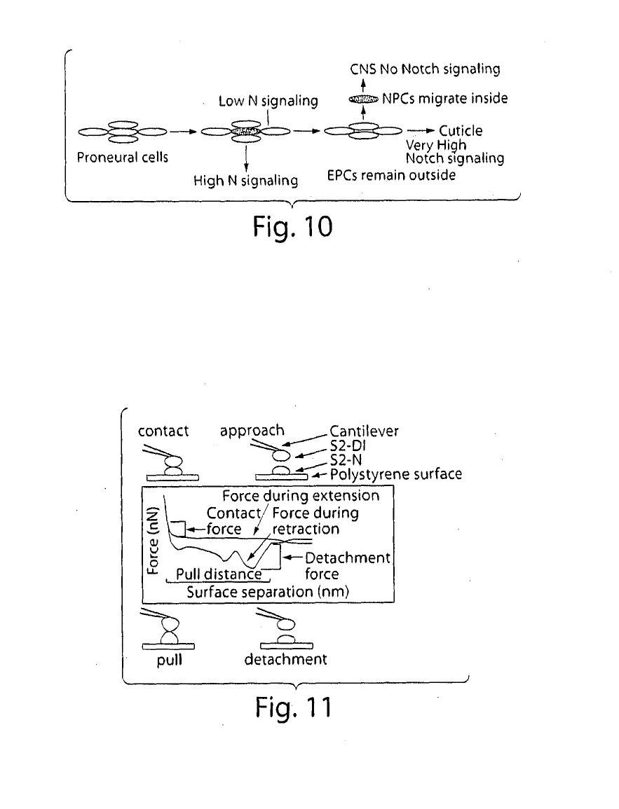

Notch signaling regulates the differentiation of the central nervous system (CNS) and the epidermis (cuticle) from clusters of stem cells called proneural cells (FIG. 10) (Artavanis-Tsakonas, S. et al., 1999. Science 284: 770-776). The proneural cells that produce a high level of Notch signaling become the epidermal precursor cells (EPCs), remain at the periphery of the embryos, further increase Notch signaling, adhere strongly to each other, and differentiate the cuticle; the proneural cells that produce a low level of Notch signaling become the neuronal precursor cells (NPCs), detach from the surrounding incipient EPCs, move inside the embryos, completely block Notch signaling, and differentiate the CNS. We use this process to introduce the distribution of NFull, NΔCterm, and NΔI during tissue differentiation. We use the word Notch to refer to all forms of Notch collectively.

Early stage NPCs at the periphery of the embryo give very strong signals only with the Nranks Abs indicating that they are enriched for NΔCterm. The neuronal precursor cells enrich for NΔCterm and NΔI. Late stage NPCs that have migrated inside the embryo give very strong signals only with the Nextra Abs, indicating that they are enriched for NΔI (aHb antibody detects the late stage NPC marker Hunchback). Hunchback signals showed that cells enriched for NΔI (shown using αN203) are NPCs. Confocal microscopy shows clearly that NΔI is enriched in and on the late stage NPCs. Both the early and late NPC stage embryos show uniform and low level of Ncterm Abs signals indicating low and uniform levels of NFull at these stages.

The commissures and the connectives (C&C, ˜axons) of the CNS gave strong signals with the Nextra Abs; the signals from all the N^{intra} Abs are the same as the surrounding ventral nerve cord (VNC) cells. This indicated that the C&C of the CNS were enriched for NΔI. The CNS was not enriched for epitopes of antibodies against Notch Intracellular regions. They were also null for Notch signaling as E(spl)C RNA is not detectable in C&C. Confocal microscopy clearly shows that the C&C of the CNS were enriched for NΔI as αN203 and αB give strong signals in them but not any of the Notch intracellular domain antibodies; the expected pattern of αHB signals rule out technical explanations such as antibody penetration, etc. The signals from the Notch intracellular domain antibodies showed a negative image of the Notch extracellular domain antibody signals. This indicates that the levels of NFull and NΔCterm were lower than the basal level in cells enriched for NΔI.

### Negative Association Between the Enrichment for NΔCterm or NΔI and Notch Signaling

The negative association between the enrichment for NΔCterm or NΔI and the low or zero levels of Notch signaling was observed at other developmental instances as well. Cells in the ventral region between the two rows of high Notch signaling cells (i.e., high E(spl)C expression) invaginate to form the precursor cells for many mesodermal and endodermal tissues. Results showed that these invaginating cells give strong signals with the Nextra Abs (αN203 and αB) and lower than basal level signals with the Ncterm Abs (α466) indicating that they are enriched for NΔI. At the end of the invagination process, E(spl) C RNA expression is lost in association with increased NΔCterm expression as indicated by the strong signals with the Nranks Abs. The levels of NΔI (α203, αB) levels and the levels of NΔCterm (αVT19) levels were negatively associated with the levels of NFull (α466) and SuH/Nintra signaling (E(spl)C). Cells with high levels of Notch signaling contained a lower than basal level of NΔCterm and NΔI indicated by the negative image of the E(spl) C RNA expression given by the Nranks Abs (αVT19) and the Nextra Abs (αN203) but not by the Ncterm Abs (α466). NΔCterm and NΔI were low in cells with high Notch signaling.

Differentiation of the sensory bristle organs on the fly thorax is regulated by Notch signaling: excess results in empty or double sockets while loss results in two or more bristles per organ (Doherty, D. et al., 1996. Genes Dev. 10: 421-34; Guo, M. et al., 1996. Neuron. 17: 27-41; Kavaler, J. et al., 1999. Development, 126(10):2261-72; Moore, A. W. et al., January 2004. Genes Dev. 18, 623-628; Okabe, M. et al., 2001. Nature, 411(6833):94-8). Over-expression of NΔCterm molecules that suppress the CSL Suppressor of Hairless and NFull expression (N¹⁷⁹¹⁻²¹⁵⁵ and N¹⁸⁹³⁻²¹⁵⁵) produces multiple bristles per organ indicating suppression of Notch signaling (Table 2; note the NFull related N^{intra} produces empty/double sockets).

TABLE 2
Numbers of flies showing phenotypes of loss
(col 4) or gain (col 5) of Notch signaling.

| | | | twin/multiple | single/double |
|---|---|---|---|---|
| si no | genotype | n | bristles | empty sockets |
| 1 | UASN^{intra}/daGal4 | 190 | 0 | 157 |
| 2 | UASN¹⁷⁹¹⁻²¹⁵⁵/daGal4 | 97 | 38 | 0 |
| 3 | UASN¹⁸⁹³⁻²¹⁵⁵/daGal4 | 159 | 84 | 0 |
| 4 | yw/daGal4 | 200 | 0 | 0 |

During wing development, the loss of Notch signaling results in Notched wings, expanded vein tips, and thick veins; the first two are prominent with reduced Notch, the last two with reduced Delta (Lindsley, D. & Zimm, G. 1992. Academic Press, New York, pp 485-499; Rulifson, E. J. & Blair, S. S. 1995. Development 121: 2813-2824). Expression of one copy of the NΔI-like molecule producing N^{Co} allele (Lyman, D. & Young, M. W. 1993. Proc. Natl. Acad. Sci. USA 90:10395-10399; Lindsley, D. & Zimm, G. 1992. Academic Press, New York, pp 485-499) in the background of wild type levels of Notch results in the production of phenotypes observed with reduced Delta. This was consistent with data from other labs that NΔI-like molecules suppress Notch signaling by titrating Delta away from NFull. The N^{Co} allele suppressed Notch signalling by the wild-type complement but not the N^{56ell} allele.

Data presented in the above two sections suggest the following model for neurogenesis. The basal low level of NFull is permissive for Notch signaling. The early stage NPCs enrich for NΔCterm that initiates the suppression of Notch signaling by titrating SuH away from NFull. NΔCterm gets converted to NΔI that titrates D1 away from NFull during the differentiation of the late NPCs into the CNS (see FIG. 9).

### Loss of Notch Signaling Results in Accumulation of NΔCterm and NΔI

N^{55ell} is a null allele of Notch (Lindsley, D. & Zirnm, G. 1992. Academic Press, New York, pp 485-499). N^{60g11} is a weak Notch signaling allele that produces an NΔCterm-like receptor (Brennan, K. et al., 1997. Genetics 147: 177-188; Lyman, D. & Young, M. W. 1993. Proc. Natl. Acad. Sci. USA 90:10395-10399). Interestingly, both N^{55e11} and N^{60g1}1 heterozygous flies showed increased levels of N45-50. Loss of Notch signalling increased NΔCterm molecules. N^{nd3} and N^{Ax59D} are temperature-sensitive weak Notch signaling alleles (Brennan, K. et al., 1997. Genetics 147: 177-188; Lyman, D. & Young, M. W. 1993. Proc. Natl. Acad. Sci. USA 90:10395-10399; Lindsley, D. & Zimm, G. 1992. Academic Press, New York, pp 485-499) carrying CADASIL-like mutations in the nd3 and the Abruptex regions, respectively (see FIG. 6). At the restrictive temperature, they also overproduced N45-50 as well as Ni60 linked to NΔI production. Loss of Notch signalling increased NI60 and NI45-50 levels. High levels of a slow migrating NΔI were also observed in these flies when the same blot is probed with one antibody after the other; note Nextra Abs detects NΔI but not Nranks Abs). The loss of Notch signalling increased high molecule weight NΔI.

In zygotic Notch null (N^{55e11}/Y) embryos, staining with all Notch antibodies ultimately disappear. But at stages just beginning to show the effects of the loss of Notch signaling, the Nextra Abs and Nranks Abs signals increase and Ncterm Abs signals decrease compared with wild type (yw) embryos. Notch null (N^{55e11}/y) embryos produced high levels of dominant negative NΔI and NI45-50 molecules. Nextra and Nranks Abs signals increase even in zygotic D1 null (D1⁻/D1⁻) embryos (note that Ncterm Abs signals were comparable to yw signals as expected). NΔI and NΔCterm levels increased in embryos deficient in lateral inhibition and SuH/N^{intra} signaling. The strong Nranks Abs (α7477) signals apparent in Delta null embryos beginning to show the effect of the loss of Notch signaling (1, 3, 5) disappear in later stage embryos (2, 4, 6) that show persistent strong Nextra Abs (αNO and αB) signals. These data indicate that loss of Notch signaling, not any particular Notch mutation, results in transient accumulation of NΔCterm and persistent accumulation of NΔI. Their accumulation might be facilitated by the lack of the carboxyl terminus required for Delta dependent and Delta independent internalization and down-regulation (Rechsteiner, M. 1988. Adv. Enzyme Regul. 27: 135-151; Wilkin, M. B. et al., 2004. Curr Biol. 14:2237-44; Bardot, B. et al., Exp. Cell Res. 304: 202-223). Both Notch null and Delta null embryos show accumulation of NΔI in distinct foci, which may be related to the Notch 3 extracellular domain accumulation in the CADASIL patients.

### Loss of Notch Signaling Leads to Disintegration of Embryos

When Notch signaling is reduced at very early stages (by reducing both the maternal and the zygotic contributions of Notch), a significant fraction of such embryos disintegrate (20-50%). We have observed this with null alleles (N55e11, ^{N264-47}) and hypomorphic alleles (N^{60g11}, N^{nd3}, and N^{Ax59D}). Experiments demonstrated that there was disintegration of maternal and zygotic Notch null embryos. The disintegration could be due to loss of Notch adhesive functions (Goode, S. et al., 1996. Development 122, 3863-3879). Thus, tissue disintegration with mutant alleles of Notch, that is a hallmark of the CADASIL disease, is also observed in flies. The alleles involved here indicate that it is due to loss of Notch signaling.

Atomic Force Microscopy (AFM) Studies Show that N^{nd3} and Ax^{59D} Receptors Bind Delta Weakly

AFM is ideal for studying cell surface molecular interactions under physiological conditions (Schabert, F. A. et al., 1995. Science 268, 92-94; Benoit, M. et al., 2000. Nat. Cell Bio. 2: 313-317; Ahimou, F. et al., 2003. Yeast 20, 25-30). It can measure the force applied to detach one surface from another, which is called the detachment force. One surface is mounted on a probe called the cantilever that is lowered onto, or retracted from, a receptacle containing the other surface. The maximum detachment force (i.e., the binding strength) is measured from the ‘force-distance graph’ generated by the deflections or bending of the cantilever. Using cantilevers containing live S2-Delta cells and Falcon plates containing live S2 cells expressing different Notch receptors, we studied the maximum detachment force and its relation to Notch signaling. The procedure is shown in FIG. 11. This is perhaps the most sensitive and developmentally relevant means available for studying Notch and Delta binding and signaling: membrane anchored on live cells, with minimal disruption and maximum controls.

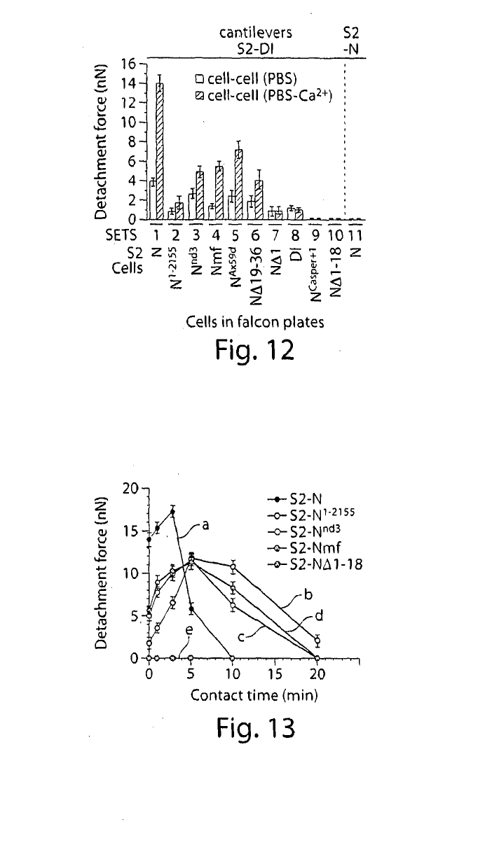

N^{nd3} and N^{Ax59D} alleles contain a CADASIL-like mutation in the nd3 and Abruptex regions, respectively (see FIG. 6). Notch 3 mutations in these regions have been shown to not affect ligand binding or Notch signaling in conventional methods. Our AFM method shows that the Delta binding strength of N^{nd3} and N^{Ax59D} receptors is 50% or less than that of the wild type NFull (FIG. 12, sets 1, 3, 5). The other sets in the figure show controls, chief among them are Notch lacking the Delta binding region (NΔ1-18) and Notch lacking the extracellular domain (N^{casper+I}): their Delta binding strength is near zero, as expected (FIG. 12, sets 9-10). Interestingly, the Delta binding strength of the NΔCterm-like N1-2155 receptor and NΔI (which have wild type sequence in the ligand binding extracellular domain) is about 10% of the wild type strength. A biochemical perspective would suggest that Delta preferentially binds NFull and that NΔCterm or NΔI can overcome this preference only by a 7 to 10× enrichment, which is consistent with the embryonic patterns we described earlier.

AFM Studies Show that N^{nd3} Receptors Generate Notch Signaling at a Lower Rate

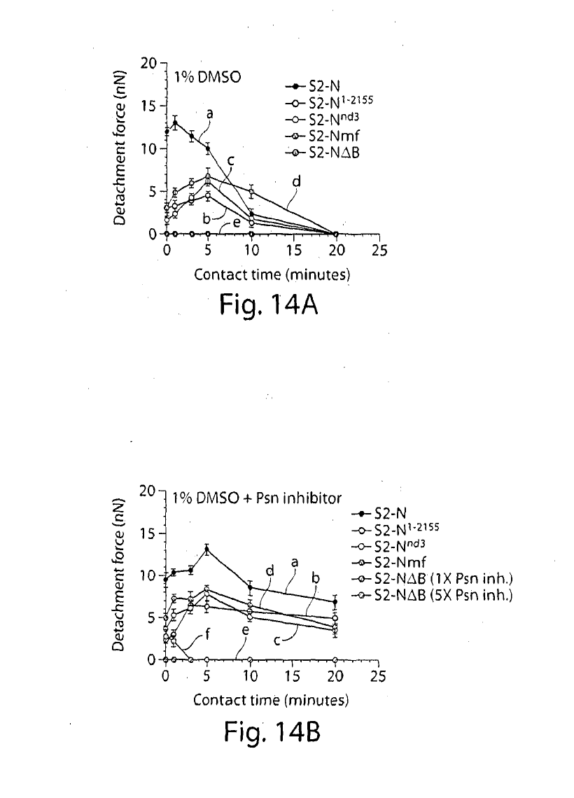

By resting the Delta cell containing cantilevers on Notch expressing cells for various times, we determined that the binding strength between NFull and Delta increases in the first few minutes and then decreases to zero, in just 10 minutes (FIG. 13, line a). The binding strength between the CADASIL-like mutation containing N^{nd3} receptor and Delta increases less rapidly and goes to zero in 20 minutes (FIG. 13 line c). The binding strength between the NΔCterm-like N¹⁻²¹⁵⁵ and Delta also increases less rapidly but does not go to zero in 20 minutes (or even 60 minutes), possibly because the NΔI produced continues to be membrane-anchored (FIG. 13, line b; see also FIG. 8). The adhesion force with S2-NΔ1-18 cell that lacks the Delta binding site is zero at all times (FIG. 13, line e). The drop in the adhesion between Notch receptors and Delta is due to Presenilin cleavage and Notch signaling as the presence of Presenilin inhibitor blocks the drop in adhesion to zero with all Notch receptors (FIG. 14). The binding strengths at time 0 and the negative slopes in both figures indicate that (1) there is a strong link between Delta binding strength and the rate Notch signaling and (2) N^{nd3} receptors bind Delta less strongly and generate Notch signaling at a lower rate than the wild type NFull. The AFM data shown also make a very interesting point. If one were to measure Delta binding strength at 20 minutes, it would lead to the erroneous conclusion that NΔCterm binds more strongly than NFull or N^{nd3} receptors; at 10 minutes, it would lead to the erroneous conclusion that NΔCterm binds the strongest, followed by N^{nd3} and NFull. It is only with measurement made at less than three minutes, will we conclude correctly that NFull binds Delta the strongest, followed by N^{nd3}, and then NΔCterm. The time range we observed here is comparable to the time taken for Notch signaling to complete in vivo.

Human Notch 3 Produces Forms that could be Related to the Production of NΔCterm- or NΔI-Like Forms

We made carboxy-terminally HA tagged versions of the wild type and two CADASIL mutant human Notch 3 receptors and expressed them in the mammalian fibroblast-like Cos 7 cells. All Notch 3 receptors, including the un-tagged receptor, produce a 65-70 kDa carboxyl terminus fragment in addition to the expected ˜97 kDa intracellular domain (N3^{TMintra}) of the hetero-dimeric receptor. Human Notch 3, Notch 1 and Notch 2 produced molecules related to the production of the NΔCterm- and NΔI-like molecules when expressed in Cos7 cells. We also made an antibody against a unique region near the RAM 23 region of Notch 3 (other antibodies are made against the carboxyl terminus). This antibody detects the above two molecules plus an additional 50 kDa molecule that apparently lacks the carboxyl terminus. Thus, the 65-70 kDa and the ˜50 kDa Notch 3 molecules are comparable to the Drosophila Ni60 and Ni45-50 molecules, raising the possibility that they are related to the production of NΔCterm- and NΔI-like molecules from human Notch 3. We observed similar molecules with human Notch 1 and Notch 2 receptors. At this time, an antibody that can distinguish NΔI-like molecules from the extracellular molecule of the hetero-dimeric receptor is not available.

Interestingly, the mammalian Notch receptors contain a putative Down-regulation Targeting Signal (DTS) sequence that is involved in Ras mediated down regulation of the C. elegans Notch homolog Lin 12 (Shaye, D. D. & I. Greenwald, I. 2002. Nature. 420:686-90). If the human Notch 3 is cleaved near this region, a ˜65 kDa carboxyl terminus fragment is expected. The 65-70 kDa carboxyl terminus fragment we see might be the human Notch 3 fragment cleaved at the DTS sequence.

### N3ΔI and N3ΔCterm Production and Suppression of Notch 3 Signaling in Human Cells

Dipeptidyl peptidase (DPPIV) is a tumor suppressor gene that blocks the tumorigenic activity of the basic FGF growth factor in prostrate cancer (Pca) cells. Tumorigenesis requires Notch in the nucleus, which means high Notch signaling (Mumm, J. S. & Kopan, R. 2000. Dev. Biol. 228: 151-165; D'Amore, P. A. & Ng, Y. S. 2002. Cell. 110: 289-92; Greenwald, I. 1998. Genes Dev. 12:1751-62; Artavanis-Tsakonas, S. et al., 1999. Science 284: 770-776; Schweisguth, F. 2004. Curr Biol. 14: R129-38; Mizutani, T. et al., 2001. Proc Natl Acad Sci USA. 98: 9026-31; Saxena, M. T. et al., 2001. Biol Chem. 276: 40268-73; Kopan, R. 2002. J. Cell Sci. 115: 1095-1097; Jeffries, S. & Capobianco, A. J. 2000. Mol. Cell. Biol. 20: 3928-41). Interestingly, Pca cells show a high level of Notch 3 in the nucleus, not Notch 1, and this is suppressed by DPPIV expression and the consequent reversal of the malignant phenotype. It was found that DPPIV re-expression in PCa suppressed nuclear Notch 3 levels. The high level of Notch 3 intracellular domain in PCa cells is reduced with DPPIV expression, concomitant with an increase in the carboxyl terminal N3cterm fragment. It was found that DPPIV promoted production of Notch 3 carboxyl terminus fragment (N3cterm) in PCa cells. N3cterm is comparable to the Drosophila Notch cterm fragments Ni52 or Ni35 (see FIG. 7). We also observed higher levels of Notch 3 equivalents of Ni45-50. N3ΔI and N3ΔCterm molecules appear to be produced in association with the loss of Notch 3 signaling in human cells, in a process comparable to the one operating in Drosophila embryos. Neuroblastoma also shows a high level of Notch 3 in the nucleus raising the possibility for the involvement of truncated Notch 3 molecules in neuronal cancer cells as well.

### A Model for the Development of CADASIL Disease Based on NΔCterm and NΔI-like Molecules

The data described above supports the following regarding the development of the CADASIL disease. Mutations in Notch 3 reduce the ligand binding strength or interfere with intracellular signal transduction. The consequent reduction in Notch 3 signaling leads to the accumulation of Notch 3 molecules lacking just the carboxyl terminus of the intracellular domain (hN3ΔCterm) that in turn leads to accumulation of Notch 3 molecules lacking most of the intracellular domain (hN3ΔI). hN3ΔI builds up slowly due to poor internalization and turnover, gradually worsening the dominant negative effect of ligand titration. Disease symptoms manifest after a threshold for the loss of Notch 3 signaling is crossed. Mice over-expressing CADASIL-like mutant receptors begin to show vascular defects at 10-12 months of age and accumulation of the Notch 3 extracellular domain at 14-16 months (Ruchoux, M. M. et al., 2003. Am J Pathol. 162:329-42). This asynchrony is not inconsistent with our hypothesis for the following reasons. One, we expect to detect hN3ΔCterm, not N3ΔI, close to the first detection of vascular defects. N3ΔI accumulation is expected later as it might be produced from hN3DCterm. Two, undetectable levels of N^{intra}/NICD is sufficient for Notch signaling in all animals (Mumm, J. S. & Kopan, R. 2000. Dev. Biol. 228: 151-165; D'Amore, P. A. & Ng, Y. S. 2002. Cell. 110: 289-92; Greenwald, I. 1998. Genes Dev. 12:1751-62; Artavanis-Tsakonas, S. et al., 1999. Science 284: 770-776; Schweisguth, F. 2004. Curr Biol. 14: R129-38; Mizutani, T. et al., 2001. Proc Natl Acad Sci USA. 98: 9026-31; Saxena, M. T. et al., 2001. Biol Chem. 276: 40268-73; Kopan, R. 2002. J. Cell Sci. 115: 1095-1097) and just a 1.5× difference in the levels of Notch, Delta, or NΔI is sufficient to produce mutant phenotypes (Lyman, D. & Young, M. W. 1993. Proc. Natl. Acad. Sci. USA 90: 10395-10399; Heitzler, P. & Simpson, P. 1991. Cell 64: 1083-1092). Three, not detected does not necessarily mean not causative. Thus, the apparent asynchrony between the detection of vascular defects and Notch 3 extracellular domain accumulation could be due to both technical and physiological reasons.

### Methods.

1) Determining whether CADASIL mutations in the three different extracellular regions of the human Notch 3 receptor reduce ligand binding strength and signaling in human cultured cells using the atomic force microscopy and pharmacologic intervention based method.
Notch and DSL ligand cDNAs: We use cDNAs for expressing the wild type human Notch 3, two CADASIL mutation containing human Notch 3, human Notch 1, human Notch 2, human Delta 1, and human Jagged 1. Notch 1 and 2 will be used for comparison and to identify any Notch 3 specific aspects. We obtain or generate by PCR, cDNAs for human Delta 2, human Jagged 2, and human Delta 3. All these ligands are used initially to pick, if possible, the strongest binding ligand of Notch 3 for our studies possibly the cognate ligand). The cDNAs of the CADASIL mutations chosen for the study are obtained from the authors or cloned using PCR. These are shown in Table 3 (asterisks show mutants to be produced by PCR).

TABLE 3
CADASIL mutations for the study.

| | CADASIL | | EGF-like | Trafficking to | Ligand | Notch | |
|---|---|---|---|---|---|---|---|
| sl no | mutation | Region | repeat | cell surface | binding | signaling | Study |
| 1 | 88-91 del* | nd3 | 2 | not det. | not det. | not det. | [60] |
| 2 | R90C | nd3 | 3 | normal | normal | normal | [36] |
| 3 | R133C | nd3 | 4 | normal | normal | normal | [36] |
| 4 | R171C | nd3 | 4 | normal | normal | not det. | [34] |
| 5 | C183R | nd3 | 5 | normal | normal | normal | [37] |
| 6 | C428C | DSL | 10 | normal | low | low | [36] |
| 7 | C455R | DSL | 11 | normal | low | low | [37] |
| 8 | C544Y | DSL | 13 | normal | normal | not det. | [34] |
| 9 | R1006C | Abruptex | 26 | normal | normal | normal | [36] |
| 10 | C1261R | other | 32 | not det. | not det. | not det. | [52] |

The CADASIL mutants are chosen (1) to provide a comparison to the results reported in the four studies using conventional in vitro methods, (2) to affect all three regions in the extracellular domain of Notch 3 (nd3, DSL binding, and Abruptex regions), (3) to include where possible gain of a cysteine, loss of a cysteine, and a deletion not affecting cysteines (with the nd3 and DSL ligand binding regions), and (4) because they were reported to be not impaired in their ability to traffic to the cell surface (except 88-91 del or C1261R, which will be dropped if they show intractable defects). We will use the cDNA for N3ΔEGFR10-11 that is deleted for the DSL binding repeats (Joutel, A. et al., 2004. Am J Hum Genet. 74:338-47. Epub 2004 Jan. 8) as our negative control for ligand binding. Similar human Notch 1 or Notch 2 molecules will be constructed for the study.

Notch and DSL ligand expression constructs. The above cDNAs are first cloned into a pUAST-HA vector we have made that places HA tags in the carboxyl terminus, and then with the HA tag cloned into the pTRE vector (Clontech) for expression from the CMV promoter placed under the control of the Tetracycline Response Element. Study of the same cell lines with and without Tetracycline (or doxycyclin) induction indicates the specificity of the response to expressed proteins and eliminate effects due to the endogenous Notch or DSL proteins. In all our assays, data with the uninduced cells serves as the baseline and all comparisons are made only with data that is significantly different from this baseline. The level of endogenous expression of all Notch receptors and DSL ligands used in the study is determined by northern blots or RT-PCR. If interference is suspected, RNAi treated cells are included in the experiments.
Stable cell lines expressing the Notch receptors and the DSL ligands. Human non-adherent Jurkat, adherent HEK 293, cultured human Vascular Smooth Muscle Cells (VSMCs) ATCC # CRL-1999 (from normal aorta), and/or primary rat aortic VSMCs (isolated and processed in collaboration with Dr. Wolfgang Dostmann's lab which works with rat VSMCs) are used to establish stable lines from the above constructs following standard procedures; primary rat VSMCs are transfected at passage 2 and experimented until passage 8 (Taylor, M. S. et al., 2004. Mol. Pharmacol. 65: 1111-1119; Dey, N. B. et al., 2005. Pharmacol. 45: 404-413)). HEK 293 cell line are also adapted to grow in suspension (Jordan, M. et al., 1998. Cytotechnology 26: 39-47). We generally prefer non-adherent or weakly adherent cells to avoid using trypsin treatment for harvesting cells as it might affect Notch or Delta molecules at the cell surface. Jurkat and HEK293 have been used to study Notch signaling (Haritunians, T. et al., 2002. Circ. Res. 90: 506-508; Karlstrom, H. P. et al., 2002. Proc. Natl. Acad. Sci. USA, 99:17119-17124; Joutel, A. et al., 2004. Am J Hum Genet. 74:338-47. Epub 2004 Jan. 8; Logeat, F. et al., 1998. Proc Natl Acad Sci USA. 95: 8108-12). VSMCs provide the in vivo context and will be used within 3-5 hours of detachment to avoid anoikis (Frisch, S. M. & Francis, H. 1994. J. Cell Bio. 124: 619-626). Alternatively, we induce expression after attachment to the cantilever or the receptacle (which can be sterilized). These would not affect our results that are relative to the wild type and controls in the same experiment. Cell surface biotinylation, western blotting, immuno-fluorescence, and/or flow cytometry are used to assess the total and cell surface protein expression. Only cell lines with matched cell surface expression of receptors or ligands will be chosen. If necessary, doxycyclin induction will be varied. We use either antibodies made against the HA tag (HA.11, Covance) or available antibodies against the extracellular and the intracellular domain of human Notch 3 (5E1 and 5G7, respectively, for western blots; BC2 and BC4, respectively for immunofluorescence that we have obtained from Dr. A. Joutel), the intracellular domain of Notch 1 (BTan 20, DHSB), the intracellular domain of Notch 2 (C651.6 DBHN, DHSB), and Jagged 1 (Ts1.15H from DHSB). Other Notch 3 antibodies are also available: P11 from Eurogentec, Belgium (against the extra-cellular region) and M20 from Santa Cruz Biotechnology (against the intracellular region).
Binding of the secreted human Jagged 1-Fc fusion ligands to the Notch receptors. To compare the AFM data with the conventional in vitro methods data, thereby with data from other studies, we determine the binding of the secreted human Jagged 1-Fc fusion protein to our wild type and mutant Notch 3 receptor expressing cells. We produce the sJ1-Fc conditioned medium using a cell expressing sJ1-Fc Jagged-Fc fusion construct (Joutel, A. et al., 2004. Am J Hum Genet. 74:338-47. Epub 2004 Jan. 8) and perform immuofluorescence based ligand binding assays exactly as described in (Joutel, A. et al., 2004. Am J Hum Genet. 74:338-47. Epub 2004 Jan. 8) and (Hicks, C. et al., 2002. J. Neurosci. Res. 68: 655-667). N3ΔEGFR10-11 expressing cells will serve as our negative control, in addition to other controls.
Binding strength between the wild type Notch receptors and DSL ligands. The AFM procedure we have developed for measuring the binding strength (i.e. detachment force) between the Drosophila Notch receptors and Delta ligand in live cells is followed with the mammalian Notch receptors and DSL ligands expressed in mammalian cells. We have the 2-3 cell lines expressing the different wild type Notch receptors (Notch 1-3), the 2-3 cell lines expressing the three Notch receptors deleted for their DSL ligand binding regions (as controls), cell lines expressing the five different ligands (Delta 1-3, Jagged 1-2), the vector alone transfected cell line, and the untransfected cell line for each type of cell (1 VSMC and 1 non VSMC selected in small scale test experiments). Protein expression is induced for 24 hours with doxycyclin. A set without doxycyclin and other controls will be processed simultaneously. Cells are harvested by shaking or gentle scraping and washed. Notch expressing cells and control cells are plated in Falcon plates at a density that ensures a uniform monolayer of cells. The ligand expressing cells are plated at a very low density and single ligand-expressing cells are picked with the lectin-coated cantilevers. The cantilevers are stored in the medium or PBS+calcium until use. If necessary, cells are attached to the cantilever prior to induction. A separate cantilever is used for each measurement. Detachment force measurements on at least 10 cells are made for each ligand-receptor pair, including for all control cells. Experiments with each cell line are repeated at least three times. Data is analyzed by the Nanoscope III program (Digital Instruments) to compare the binding strengths (detachment forces) between the various samples.

The Y-intercept and the maximum detachment forces are plotted for the determination of adhesion/binding strengths between the five putative ligands and the wild type Notch receptors. The binding strength between the ligands and the Notch receptors deleted for the DSL binding region serves as our baseline measurement. All assessment are based on values significantly different from these baselines. If differences exist between the different receptor-ligand pairs, the strongest binding ligand is chosen for each receptor. If not, we choose the most convenient one(s) in terms of the tools available (antibodies, etc.), or Jagged 1 for Notch 3 as it thought to be this receptor's cognate ligand.

Binding strength between the mutant Notch 3 receptors and their ligand. The procedure and experiments described above are repeated with each of the 10 mutants. The binding strengths of the mutant receptors are tested for statistically significant differences from the baseline controls as well as from the wild type receptor in each experiment. All differences found are be expressed relative to the wild type receptor. All interpretations of the differences include the effect of mutations on protein modifications and conformation.
Determination of the rate of signaling by the wild type and mutant Notch 3 receptors. The wild type and all mutant Notch 3 receptors are used to study the change in detachment force over 0, 3, 5, 10, 20, 40, and 60 minutes. Data from all experiments (controls, treatments, and replications) is was plotted. The same experiments are repeated in the presence of the Presenilin/g-secretase inhibitors (DFK 167) to determine and confirm that the changes are due to the Presenilin/g-secretase cleavage in the manner described above herein. The most informative time points are identified and the differences between the receptors at these times and the differences in the negative slope of the detachment force curve (in the absence of inhibitors) are tested for statistical significance. These two values measure the most proximate or immediate Notch 3 signaling response to ligand binding and represent the intrinsic signaling capacity of the different receptors, minimally affected by any response mounted by the cells.

If significant differences are found between the wild-type receptor and the mutants, we examine these differences in the conventional assays at the incubation time shown by AFM to be the peak for Notch 3 signaling (as well as a low point for comparison). For this purpose, we use co-transfection with the HES 1 promoter-luciferase or RBP/JK-luciferase reporter genes (along with the β-galactosidase gene for signal standardization) used by many other labs in conventional assays of Notch 3 and Notch signaling in mammalian and human cell lines (Kopan, R. 2002. J. Cell Sci. 115: 1095-1097; Haritunians, T. et al., 2002. Circ. Res. 90: 506-508; Karlstrom, H. P. et al., 2002. Proc. Natl. Acad. Sci. USA, 99:17119-17124; Joutel, A. et al., 2004. Am J Hum Genet. 74:338-47. Epub 2004 Jan. 8; Peters, N. et al., 2004. Exp Cell Res. 299: 454-64; Shawber, C. D. et al., 1996. Development 122: 3765-3773; Hsieh, J. et al., 1996. Mol. Cell. Biol. 16: 952-959; Beatus, P. et al., 1999. Development 126: 3925-3935; Jarriault, S. et al., 1998. Mol. Cell. Biol. 18: 7423-7431). If necessary, we also monitor the levels of the endogenous HES 1 and HES 5 shown to be responsive to all Notch signaling (Jarriault, S. et al., 1998. Mol. Cell. Biol. 18: 7423-7431; Jarriault, S. et al., 1995. Nature 377: 355-358). We also examine the expression of Hairy related transcription factors (HRT 1-3) that is responsive to Notch 3 signaling in rat VSMCs (Wang, W. et al., 2002. J. Biol. Chem. 277: 23165-23171).

2) Investigation to determine if NΔCterm-like and NΔI-like molecules are indeed produced from the human Notch 3 receptor expressed in human cultured cells and to examine if their levels are affected by CADASIL mutations using conventional cyto-chemical and molecular procedures.
Antibodies. We make one set of antibodies in rats against the poorly conserved region between the transmembrane domain and the Ram 23 region of human Notch 3 (amino acids 1665-1782), Notch 1 (amino acids 1792-1859), and Notch 2 (amino acids 1746-1816). Antibodies identifying one Notch receptor and not others are not essential; our inducible system distinguishes transgenic proteins from endogenous proteins. One antibody is made in chickens against the highly conserved Ankyrin repeats that will detect this region in all human Notch receptors (we obtained excellent chicken antibodies against the Drosophila Ankyrin repeats). These two sets of antibodies help determine if NΔCterm related molecules are produced from the human Notch 3, Notch 2, and Notch 1 receptors. A third set of antibodies is made against the region between the transmembrane domain and the S1 cleavage site in human Notch 3 (amino acids 1572-1643), Notch 1 (amino acids 1670-1733), and Notch 2 (amino acids 1612-1677). We make antibodies against a suitable 25 amino acid peptide within these regions. This set of antibodies distinguishes NΔI-like molecules from the extracellular domain molecule of the heterodimeric receptor. We include Notch 1 or Notch 2 in these studies to determine the generality of our observations as all Notch receptors appear to function in a similar manner. We also produce Notch 3, Notch 1, and Notch 2 molecules with the HA tag placed between the stop-transfer signal after the transmembrane domain and the putative DTS sequence to provide independent confirmation of our results and serve as a back-up strategy should for any reason the Notch specific antibody approach fails. Tags in this region are known to work (Struhl, G. & Adachi, A. 2000. Mol. Cell 6: 625-636).
Production of NΔCterm and NΔI related molecules. (1) All stable cell lines expressing Notch receptors are examined for production of all the intracellular and extracellular domain molecules (similar to Drosophila Ni60, Ni52, Ni35, Ni45-50, Ni32, and NΔI molecules or others). These molecules are analyzed in detail using combinations of antibodies in immuno-precipitation and western blotting to get a fairly good idea about their structures. Since we use HA tagged molecules, the intracellular fragments can be purified and their termini sequenced, if necessary. (2) Using cell surface biotinylation and streptavidin or Notch antibody immuno-precipitation experiments, we determine if any of the intracellular domain molecules are linked to the Notch receptor at the cell surface. N^{TMintra}/NICD recovery serves as a positive control. (3) Using Delta or Jagged immuno-precipitation experiments, we determine if the intracellular domain molecules of interest are linked to the Notch molecules that bind the ligands. N^{TMintra}/NICD molecules will serve as positive controls. For these experiments, we follow the same procedure used by others to study Notch 3 signaling (co-culturing Notch-expressing and ligand-expressing cells), but with the optimal incubation times identified in our AFM studies. We have developed a method using membrane impermeable or permeable, reversible or irreversible, cross-linkers for preferentially recovering molecules interacting at the cell surface or inside the cells (Wesley, C. 1999. Mol. Cell. Biol. 19: 5743-5758). We use this procedure, along with the panel of region specific antibodies, to determine the structure of all the receptors that bind ligands. (4) Using immuno-fluorescent experiments with antibodies against the different regions of the Notch receptors we determine the subcellular distribution of the different forms, in the absence and presence of ligands. We also determine if the extracellular domain, S1 site to TM, TM to Ram23, Ankyrin repeats, and the carboxyl terminus epitopes of Notch co-localize or not. (5) We determine if the different intracellular molecules change their levels in response to ligand treatment. (6) In all the above experiments, we use a sample of Notch 3 receptors with CADASIL mutations to determine if these mutations affect the levels of the truncated molecules and the interaction of these molecules with ligands. (7) We express any interesting molecules we identify, and molecules resembling the Drosophila NΔI, Ni45-50, and Ni32 in the VSMCs and HEK293 cells and determine if they reduce Notch 3 signaling, reduce the levels of endogenous Notch receptors, and disrupt cell adhesion, as such molecules do in Drosophila.
Stability of the different Notch receptor molecules. Using metabolic labeling experiments (per (Hicks, C. et al., 2002. J. Neurosci. Res. 68: 655-667)), we determine if the different molecules of interest exhibit any differences in their turn over, in the presence and absence of ligands. Using immunofluorescence and immunoprecipitation experiments (described above), we determine if the stability of the different Notch 3 molecules is linked to their association with the ligand. We expect molecules comparable to NΔCterm and NΔI molecules to be more stable than other Notch 3 molecules.

With the above experiments, we determine (1) if molecules resembling NΔCterm and NΔI are produced from Notch 3 as suggested by our previous data, (2) if they have activities similar to those of comparable Drosophila molecules, and (3) if their production is altered with CADASIL mutations.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

1. A method for identifying the level of Notch signaling in a cell or tissue comprising:
determining an amount of truncated Notch polypeptide of the cell or tissue,
comparing the amount of truncated Notch polypeptide of the cell or tissue to an amount of truncated Notch polypeptide of a control cell or tissue, wherein a higher or lower amount of truncated Notch polypeptide of the cell or tissue compared to the control cell or tissue identifies the cell or tissue as having a different level of Notch signaling than the level of Notch signaling of the control cell or tissue.

2. The method of claim 1, wherein a higher amount of truncated Notch polypeptide in the cell or tissue compared to the control cell or tissue identifies the cell or tissue as having a lower level of Notch signaling than the control cell or tissue.

3. The method of claim 1, wherein a lower amount of truncated Notch polypeptide in the cell or tissue compared to the control cell or tissue identifies the cell or tissue as having a higher level of Notch signaling than the control cell or tissue.

4. The method of claim 1, wherein determining the amount of truncated Notch polypeptide comprises the use of immunodetection methods.

5. The method of claim 1, wherein the amount of truncated Notch polypeptide is determined by
contacting the cell or tissue with one or more antibodies or antigen-binding fragments thereof that specifically bind to one or more domain(s) present in a truncated Notch polypeptide and one or more antibodies or antigen-binding fragments thereof, that specifically bind to the C-terminal domain of a Notch polypeptide,
detecting the level of binding of the antibodies or antigen-binding fragments thereof to the cell or tissue, and
comparing the level of binding of the antibodies or antigen-binding fragments thereof that bind to domain(s) present in the truncated Notch polypeptide to the level of binding of the antibodies or antigen-binding fragments thereof that bind to the C-terminal domain of the Notch polypeptide as a determination of the amount of truncated Notch polypeptide of the cell or tissue.

6. The method of claim 5, wherein the one or more antibodies or antigen-binding fragments thereof that specifically bind to a domain present in a truncated Notch polypeptide is an antibody or antigen-binding fragment thereof that specifically binds either the extracellular domain of the Notch polypeptide or a Ram23+Ankyrin domain of the Notch polypeptide.

7. The method of claim 5, wherein the cell or tissue is contacted with least one antibody or antigen-binding fragment thereof that specifically binds to the C-terminal domain of a Notch polypeptide and at least one antibody or antigen-binding fragment thereof that specifically binds to the extracellular Notch polypeptide domain or to the Ram23+Ankyrin Notch polypeptide domain.

8-18. (canceled)

19. The method of claim 5 wherein the antibodies or antigen-binding fragments thereof, are detectably labeled.

20. (canceled)

21. A method for identifying the level of Notch signaling in a cell or tissue comprising:
determining a ratio of an amount of truncated Notch polypeptide of the cell or tissue to an amount of full-length Notch polypeptide of the cell or tissue,
comparing the ratio of the amount of truncated Notch polypeptide to the amount of full-length polypeptide of the cell or tissue to a ratio of the amount of truncated Notch polypeptide to the amount of full-length Notch polypeptide of a control cell or tissue, wherein a different ratio of the cell or tissue compared to the ratio of the control cell or tissue identifies the cell or tissue as having a different level of Notch signaling than the level of Notch signaling of the control cell or tissue.

22. The method of claim 21, wherein a higher ratio of the amount of truncated Notch polypeptide to the amount of full-length polypeptide of the cell or tissue compared to the ratio of the amount of truncated Notch polypeptide to the amount of full-length polypeptide of the control cell or tissue identifies the cell or tissue as having a lower level of Notch signaling than the control cell or tissue.

23. The method of claim 21, wherein a lower ratio of amount of truncated Notch polypeptide to the amount of full-length polypeptide of the cell or tissue compared to the ratio of the amount of truncated Notch polypeptide to the amount of full-length polypeptide of the control cell or tissue identifies the cell or tissue as having a higher level of Notch signaling than the control cell or tissue.

24-27. (canceled)

28. The method of claim 21, wherein determining the ratio of the amount of truncated Notch polypeptide of the cell or tissue to the amount of full-length Notch polypeptide of the cell or tissue comprises the use of immunodetection methods.

29. The method of claim 21, wherein determining the ratio of the amount of the truncated Notch polypeptide of the cell or tissue to the amount of the full-length Notch polypeptide of the cell or tissue comprises:
contacting the cell or tissue with one or more antibodies or antigen-binding fragments thereof that specifically bind to one or more domain(s) present in the truncated Notch polypeptide,
contacting the cell or tissue with one or more antibodies or antigen-binding fragments thereof that specifically bind the C-terminal domain of the Notch polypeptide;
detecting the level of binding of the truncated Notch polypeptide and Notch polypeptide C-terminal antibodies or antigen-binding fragments thereof in the cell or tissue, and
comparing the level of binding of the truncated Notch polypeptide and Notch polypeptide C-terminal antibodies or antigen-binding fragments thereof to the cell or tissue to determine the ratio of the truncated and full-length Notch polypeptide in the cell or tissue.

30. The method of claim 29, wherein the cell or tissue is contacted with least one antibody or antigen-binding fragment thereof that specifically binds to the C-terminal domain of a Notch polypeptide and at least one antibody or antigen-binding fragment thereof that specifically binds to the extracellular Notch polypeptide domain or to the Ram23+Ankyrin Notch polypeptide domain.

31-37. (canceled)

38. The method of claim 29 wherein the antibodies or antigen-binding fragments thereof are detectably labeled.

39-113. (canceled)

114. A method for identifying a change in the level of Notch signaling in a subject comprising:
determining in a first biological sample obtained from the subject an amount of truncated Notch polypeptide,
determining in a second biological sample obtained from the subject at a time later than the first biological sample an amount of truncated Notch polypeptide,
comparing the level of truncated Notch polypeptide in the first and second samples, wherein a difference in the level of truncated Notch polypeptide in the first sample compared to the level of truncated Notch polypeptide in the second sample identifies a change in the level of Notch signaling in the subject.

115. The method of claim 114, wherein determining the amount of truncated Notch polypeptide comprises the use of immunodetection methods.

116. The method of claim 114, wherein the level of truncated Notch polypeptide is determined by
contacting the biological sample with one or more antibodies or antigen-binding fragments thereof that specifically bind to one or more domain(s) present in a truncated Notch polypeptide and one or more antibodies or antigen-binding fragments thereof that specifically bind to the C-terminal domain of a Notch polypeptide,
detecting the level of binding of the antibodies or antigen-binding fragments thereof and
comparing the level of binding of the antibodies or antigen-binding fragments thereof that specifically the domain(s) present in a truncated Notch polypeptide to the level of binding of the antibodies or antigen-binding fragments thereof that specifically bind to the C-terminal domain of the Notch polypeptide as a measure of the level of truncated Notch polypeptide in the biological sample.

117. The method of claim 114, wherein a higher level of truncated Notch polypeptide in the first biological sample compared to the second biological sample identifies a higher level of Notch signaling in the second biological sample than in the first biological sample.

118. The method of claim 114, wherein a lower level of truncated Notch polypeptide in the first biological sample compared to the second biological sample identifies a lower level of Notch signaling in the second biological sample than in the first biological sample.

119-179. (canceled)


SEQUENCE LISTING (5 sequences)

SEQ ID NO: 1 - protein, 2556 aa, Homo sapiens
  misc_feature (891)..(891): Xaa can be any naturally occurring amino acid
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPRLCRHGGTCHNEVGSYRCVCRATHTGPNCERPYVPCSPSPCQNGGTCRPTGDVTHECACLPGFTGQNCEENIDDCPGNNCKNGGACVDGVNTYNCPCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCMCMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRLRGTCQDPDNAYLCFCLKGTTGPNCEINLDDCASSPCDSGTCLDKIDGYECACEPGYTGSMCNSNIDECAGNPCHNGGTCEDGINGFTCRCPEGYHDPTCLSEVNECNSNPCVHGACRDSLNGYKCDCDPGWSGTNCDINNNECESNPCVNGGTCKDMTSGIVCTCREGFSGPNCQTNINECASNPCLNKGTCIDDVAGYKCNCLLPYTGATCEVVLAPCAPSPCRNGGECRQSEDYESFSCVCPTAGAKGQTCEVDINECVLSPCRHGASCQNTHGXYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHVVNECDSRPCLLGGTCQDGRGLHRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 2 - protein, 2471 aa, Homo sapiens
MPALRPALLWALLALWLCCATPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCLQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDHMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 3 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPVAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 4 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 5 - protein, 2703 aa, Drosophila melanogaster
MQSQRSRRRSRAPNTWICFWINKMHAVASLPASLPLLLLTLAFANLPNTVRGTDTALVAASCTSVGCQNGGTCVTQLNGKTYCACDSHYVGDYCEHRNPCNSMRCQNGGTCQVTFRNGRPGISCKCPLGFDESLCEIAVPNACDHVTCLNGGTCQLKTLEEYTCACANGYTGERCETKNLCASSPCRNGATCTALAGSSSFTCSCPPGFTGDTCSYDIEECQSNPCKYGGTCVNTHGSYQCMCPTGYTGKDCDTKYKPCSPSPCQNGGICRSNGLSYECKCPKGFEGKNCEQNYDDCLGHLCQNGGTCIDGISDYTCRCPPNFTGRFCQDDVDECAQRDHPVCQNGATCTNTHGSYSCICVNGWAGLDCSNNTDDCKQAACFYGATCIDGVGSFYCQCTKGKTGLLCHLDDACTSNPCHADAICDTSPINGSYACSCATGYKGVDCSEDIDECDQGSPCEHNGICVNTPGSYRCNCSQGFTGPRCETNINECESHPCQNEGSCLDDPGTFRCVCMPGFTGTQCEIDIDECQSNPCLNDGTCHDKINGFKCSCALGFTGARCQINIDDCQSQPCRNRGICHDSIAGYSCECPPGYTGTSCEININDCDSNPCHRGKCIDDVNSFKCLCDPGYTGYICQKQINECESNPCQFDGHCQDRVGSYYCQCQAGTSGKNCEVNVNECHSNPCNNGATCIDGINSYKCQCVPGFTGQHCEKNVDECISSPCANNGVCIDQVNGYKCECPRGFYDAHCLSDVDECASNPCVNEGRCEDGINEFICHCPPGYTGKRCELDIDECSSNPCQHGGTCYDKLNAFSCQCMPGYTGQKCETNIDDCVTNPCGNGGTCIDKVNGYKCVCKVPFTGRDCESKMDPCASNRCKNEAKCTPSSNFLDFSCTCKLGYTGRYCDEDIDECSLSSPCRNGASCLNVPGSYRCLCTKGYEGRDCAINTDDCASFPCQNGGTCLDGIGDYSCLCVDGFDGKHCETDINECLSQPCQNGATCSQYVNSYTCTCPLGFSGINCQTNDEDCTESSCLNGGSCIDGINGYNCSCLAGYSGANCQYKLNKCDSNPCLNGATCHEQNNEYTCHCPSGFTGKQCSEYVDWCGQSPCENGATCSQMKHQFSCKCSAGWTGKLCDVQTISCQDAADRKGLSLRQLCNNGTCKDYGNSHVCYCSQGYAGSYCQKEIDECQSQPCQNGGTCRDLIGAYECQCRQGFQGQNCELNIDDCAPNPCQNGGTCHDRVMNFSCSCPPGTMGIICEINKDDCKPGACHNNGSCIDRVGGFECVCQPGFVGARCEGDINECLSNPCSNAGTLDCVQLVNNYHCNCRPGHMGRHCEHKVDFCAQSPCQNGGNCNIRQSGHHCICNNGFYGKNCELSGQDCDSNPCRVGNCVVADEGFGYRCECPRGTLGEHCEIDTLDECSPNPCAQGAACEDLLGDYECLCPSKWKGKRCDIYDANYPGWNGGSGSGNDRYAADLEQQRAMCDKRGCTEKQGNGICDSDCNTYACNFDGNDCSLGINPWANCTANECWNKFKNGKCNEECNNAACHYDGHDCERKLKSCDSLFDAYCQKHYGDGFCDYGCNNAECSWDGLDCENKTQSPVLAEGAMSVVMLMNVEAFREIQAQFLRNMSHMLRTTVRLKKDALGHDIIINWKDNVRVPEIEDTDFARKNKILYTQQVHQTGIQIYLEIDNRKCTECFTHAVEAAEFLAATAAKHQLRNDFQIHSVRGIKNPGDEDNGEPPANVKYVITGIILVIIALAFFGMVLSTQRKRAHGVTWFPEGFRAPAAVMSRRRRDPHGQEMRNLNKQVAMQSQGVGQPGAHWSDDESDMPLPKRQRSDPVSGVGLGNNGGYASDHTMVSEYEEADQRVWSQAHLDVVDVRAIMTPPAHQDGGKHDVDARGPCGLTPLMIAAVRGGGLDTGEDIENNEDSTAQVISDLLAQGAELNATMDKTGETSLHLAARFARADAAKRLLDAGADANCQDNTGRTPLHAAVAADAMGVFQILLRNRATNLNARMHDGTTPLILAARLAIEGMVEDLITADADINAADNSGKTALHWAAAVNNTEAVNILLMHHANRDAQDDKDETPLFLAAREGSYEACKALLDNFANREITDHMDRLPRDVASERLHHDIVRLLDEHVPRSPQMLSMTPQAMIGSPPPGQQQPQLITQPTVISAGNGGNNGNGNASGKQSNQTAKQKAAKKAKLIEGSPDNGLDATGSLRRKASSKKTSAASKKAANLNGLNPGQLTGGVSGVPGVPPTNSAAQAAAAAAAAVAAMSHELEGSPVGVGMGGNLPSPYDTSSMYSNAMAAPLANGNPNTGAKQPPSYEDCIKNAQSMQSLQGNGLDMIKLDNYAYSMGSPFQQELLNGQGLGMNGNGQRNGVGPGVLPGGLCGMGGLSGAGNGNSHEQGLSPPYSNQSPPHSVQSSLALSPHAYLGSPSPAKSRPSLPTSPTHIQAMRHATQQKQFGGSNLNSLLGGANGGGVVGGGGGGGGGVGQGPQNSPVSLGIISPTGSDMGIMLAPPQSSKNSAIMQTISPQQQQQQQQQQQQQHQQQQQQQQQQQQQQQQQLGGLEFGSAGLDLNGFCGSPDSFHSGQMNPPSIQSSMSGSSPSTNMLSPSSQHNQQAFYQYLTPSSQHSGGHTPQHLVQTLDSYPTPSPESPGHWSSSSPRSNSDWSEGVQSPAANNLYISGGHQANKGSEAIYI